(19) United States
(12) Patent Application Publication
(10) Pub. No.: US 2024/0409946 A1
(43) Pub. Date: Dec. 12, 2024

(54) MICRORNA TARGETING REGENERATIVE TREATMENTS FOR HEART FAILURE

(71) Applicant: Jaan Biotherapeutics LLC, San Diego, CA (US)

(72) Inventor: Bhawanjit Kaur Brar, La Jolla, CA (US)

(21) Appl. No.: 18/807,909

(22) Filed: Aug. 16, 2024

Related U.S. Application Data
(63) Continuation of application No. PCT/US2023/062775, filed on Feb. 16, 2023.
(60) Provisional application No. 63/311,448, filed on Feb. 17, 2022.

(51) Int. Cl. C12N 15/113; A61K 48/00

(52) U.S. Cl. CPC C12N 15/1137; A61K 48/0058; C12N 2310/141; C12N 2750/14143

(57) ABSTRACT

Disclosed herein include methods for preventing, inhibiting, reducing, or treating cardiac ischemic injury. The method comprises administering a therapeutic composition comprising a plurality of microRNA (miR) antagonists to a subject before, during, and/or after a cardiac ischemic event. The method can comprise administration of the therapeutic composition as two doses separated by an interval.

### RELATED APPLICATIONS

This application claims the benefit under 35 U.S.C. § 119(e) of U.S. Provisional Patent Application Ser. No. 63/311,448, filed Feb. 17, 2022, the content of this related application is incorporated herein by reference in its entirety for all purposes.

### STATEMENT REGARDING FEDERALLY SPONSORED R&D

This invention was made with government support under grant No. 1R43HL154884-01 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 68BX-312172-WO_Sequence Listing, created Feb. 15, 2023, which is 43 kilobytes in size. The information in the electronic format of the Sequence Listing is incorporated herein by reference in its entirety.

### BACKGROUND

### Field

The present disclosure relates generally to the field of heart muscle regeneration. More particularly, disclosed herein are methods of preventing, inhibiting, reducing, or treating cardiac ischemic reperfusion injury.

### Description of the Related Art

Ischemic heart disease (IHD) is the single largest cause of death in the world and is commonly a consequence of an acute myocardial infarction (MI). Patients afflicted with IHD exhibit significant morbidity and a poor prognosis. Thus, a significant unmet medical need exists to develop new treatment approaches that enhance endogenous cardiomyocyte regeneration, with consequential mitigation of myocardial dysfunction and prevention of the transition to HF.

### SUMMARY

Disclosed herein include methods of preventing, inhibiting, reducing, or treating ischemic heart injury. In some embodiments, the method comprises administering a therapeutic composition to a subject before, during, and/or after a cardiac ischemic event. In some embodiments, the therapeutic composition is administrated in a first dose and a second dose separated by a dosing interval.

Disclosed herein include methods of inducing cardiomyocyte regeneration, cardiac repair, vasculogenesis and/or cardiomyocyte differentiation. In some embodiments, the method comprises administering a therapeutic composition to a subject. In some embodiments, the therapeutic composition is administrated in a first dose and a second dose separated by a dosing interval.

The therapeutic composition can comprise a nucleotide sequence having at least 80% identity, at least 90% identity, or at least 95% identity to SEQ ID NO: 1. In some embodiments, the therapeutic composition comprises a nucleotide sequence of SEQ ID NO: 1, or a nucleotide sequence having no more than ten mismatches to the nucleotide sequence of SEQ ID NO: 1. In some embodiments, the therapeutic composition comprises at least two nucleotide sequences selected from: (i) a nucleotide sequence having at least 80% identity, at least 90% identity, or at least 95% identity to SEQ ID NOs: 14-18, (ii) a nucleotide sequence having at least 80% identity, at least 90% identity, or at least 95% identity to SEQ ID NOs: 19-23, (iii) a nucleotide sequence having at least 80% identity, at least 90% identity, or at least 95% identity to SEQ ID NOs: 24-31, and (iv) a nucleotide sequence having at least 80% identity, at least 90% identity, or at least 95% identity to SEQ ID NOs: 26-33. In some embodiments, the therapeutic composition comprises a nucleotide sequence of SEQ ID NOs: 17 and 26, or a nucleotide sequence having no more than ten mismatches to the nucleotide sequence of SEQ ID NOs: 17 and 26.

In some embodiments, the first dose and/or the second dose are administrated through routes including intracardiac, intramuscular, intravaginal, intravenous, intra-myocardial, intra-ventricular, subcutaneous, systemic, intra-coronary, intraperitoneal, subcutaneous, epicutaneous, intradermal, rectal, intraocular, pulmonary, intracranial, intraosseous, oral, buccal, or nasal. In some embodiments, the first dose and the second dose are administrated through the same or different routes. In some embodiments, the first dose is administrated through intracardiac (IC) infusion. In some embodiments, the first dose is administrated at a rate of 0.1/mL/min-10/mL/min. In some embodiments, the first dose is administrated at a rate of 1/mL/min. In some embodiments, the second dose is administrated intravenously (IV).

In some embodiments, the first dose is administrated after the cardiac ischemic event. In some embodiments, first dose is administrated no more than 30 days after the cardiac ischemic event. In some embodiments, the first dose is administrated before the cardiac ischemic event. In some embodiments, the first dose is administrated during the cardiac ischemic event. In some embodiments, the second dose is administrated at least 1 day after the first dose. In some embodiments, the second dose is administrated at least 30 days after the first dose. In some embodiments, the second dose is administrated before and/or after the first dose is cleared from the subject. In some embodiments, the second dose is administrated when at least 50% of the first dose is cleared from the subject. In some embodiments, the second dose is administrated at a timing that it does not instigate immune response.

In some embodiments, the first dose is administered at a dose of at least 1×10¹⁰ vg/kg. In some embodiments, the first dose is administered at a dose of 2.5×10¹² vg/kg. In some embodiments, the second dose is at least 50% of the first dose. In some embodiments, the second dose is administered at a dose of at least 1×10¹⁰ vg/kg. In some embodiments, the second dose is about 1.7×10¹² vg/kg. In some embodiments, the second dose is administrated at the same total amount per subject as the first dose.

In some embodiments, the therapeutic composition comprises a viral and/or non-viral vector comprising the nucleotide sequence. In some embodiments, the therapeutic composition comprises a viral vector. In some embodiments, the viral vector is selected from the group consisting of adenovirus vectors, adeno-associated virus (AAV) vectors, retrovirus vectors, lentiviral vectors, herpes virus vectors, phages, and poxvirus vectors. In some embodiments, the viral vector is AAV. In some embodiments, the viral vector is selected from the group consisting of AAV 1, AAV 2, AAV 3, AAV 4, AAV 5, AAV 6, AAV 7, AAV 8, AAV 9, and AAV 10 and chimeric AAV derived thereof. In some embodiments, the viral vector is AAV2/9

In some embodiments, the therapeutic composition reduces creatine kinase (CK) and lactate dehydrogenase (LDH) levels, left ventricle (LV) cavity volume, size of scar, blood urea nitrogen (BUN) level, blood urea nitrogen/creatine ratios and anterior wall thickness; and increases LV ejection fraction and LV mass, LV wall displacement/motion, albumin/globulin (A/G) ratios, percentage of lymphocytes, septal and lateral walls thickness. In some embodiments, the CK and LDH levels, left ventricle (LV) cavity volume, size of scar, blood urea nitrogen (BUN) level, blood urea nitrogen/creatine ratios and/or anterior wall thickness are reduced by at least 5%, at least 10%, at least 20%, at least 30%, or at least 50% compared to the CK and LDH levels, left ventricle (LV) cavity volume, size of scar, blood urea nitrogen (BUN) level, blood urea nitrogen/creatine ratios and/or anterior wall thickness of the subject prior to administration with the therapeutic composition. In some embodiments, the LV ejection fraction and LV mass, LV wall displacement/motion, albumin/globulin (A/G) ratios, percentage of lymphocytes, septal and/or lateral walls thickness are increased by at least 5%, at least 10%, at least 20%, at least 30%, or at least 50% compared to the LV ejection fraction and LV mass, LV wall displacement/motion, albumin/globulin (A/G) ratios, percentage of lymphocytes, septal and/or lateral walls thickness of the subject prior to administration with the therapeutic composition. In some embodiments, the second dose of the therapeutic composition reduces CK and LDH levels, left ventricle (LV) cavity volume, size of scar, blood urea nitrogen (BUN) level, blood urea nitrogen/creatine ratios and anterior wall thickness; and increases LV ejection fraction and LV mass, LV wall displacement/motion, albumin/globulin (A/G) ratios, percentage of lymphocytes, septal and lateral walls thickness. In some embodiments, the CK and LDH levels, left ventricle (LV) cavity volume, size of scar, blood urea nitrogen (BUN) level, blood urea nitrogen/creatine ratios and/or anterior wall thickness are reduced by at least 5%, at least 10%, at least 20%, at least 30%, or at least 50% compared to the creatine kinase (CK) and LDH levels, left ventricle (LV) cavity volume, size of scar, blood urea nitrogen (BUN) level, blood urea nitrogen/creatine ratios and/or anterior wall thickness of the subject prior to administration of the second dose of the therapeutic composition. In some embodiments, the LV ejection fraction and LV mass, LV wall displacement/motion, albumin/globulin (A/G) ratios, percentage of lymphocytes, septal and/or lateral walls thickness are increased by at least 5%, at least 10%, at least 20%, at least 30%, or at least 50% compared to the LV ejection fraction and LV mass, LV wall displacement/motion, albumin/globulin (A/G) ratios, percentage of lymphocytes, septal and/or lateral walls thickness of the subject prior to administration of the second dose of the therapeutic composition.

In some embodiments, the therapeutic composition does not cause arrhythmias, a reduction in a body weight of the subject, liver toxicity, detection of red blood cells in urinalysis, and deviation of hematology values, prothrombin time (PT), activated partial thromboplastin time (APTT), peripheral oxygen saturation (SpO₂), body temperature end-tidal (ET) CO₂ and CO₂ reduction reaction (RR) from clinical reference ranges. In some embodiments, a lack of liver toxicity is measured by the level of liver enzymes selected from alanine transferase levels (ALT), alkaline phosphatase levels (ALP), aspartate aminotransferase levels (AST), total bilirubin levels (TBIL), and direct bilirubin levels (DBIL). In some embodiments, the hematology values include percentages of reticulocytes, monocytes, eosinophils, and basophils; counts of platelet, red blood cell, lymphocytes, monocytes, eosinophils, and basophils; and size and shape of erythrocytes and red blood cells.

In some embodiments, ischemic heart injury is myocardial infarction, coronary artery bypass grafting (CABG), cardiac bypass surgery, cardiac transplantation, angioplasty, vascular interventional procedure employing a stent, laser catheter, atherectomy catheter, angioscopy device, beta or gamma radiation catheter, rotational atherectomy device, coated stent, radioactive balloon, heatable wire, heatable balloon, biodegradable stent strut, a biodegradable sleeve, or any combination thereof. In some embodiments, ischemic heart injury is cardiac ischemic reperfusion injury. In some embodiments, the cardiac ischemic reperfusion injury comprises cardiac ischemic damage, cardiac reperfusion injury, or a combination thereof.

In some embodiments, the nucleotide sequence comprises one or more chemical modifications. In some embodiments, the nucleotide sequence comprises one or more chemical modifications selected from a modified internucleoside linkage, a modified nucleotide, and a modified sugar moiety, and combinations thereof. In some embodiments, the one or more chemical modifications comprise a modified internucleoside linkage. In some embodiments, the modified internucleoside linkage is selected from a phosphorothioate, 2′-0 methoxyethyl (MOE), 2′-fluoro, alkylphosphonate, phosphorodithioate, alkylphosphonothioate, phosphoramidate, carbamate, carbonate, phosphate triester, acetamidate, carboxymethyl ester, and combinations thereof. In some embodiments, the modified internucleoside linkage comprises a phosphorothioate internucleoside linkage. In some embodiments, at least one of the one or more chemical modifications comprises a modified nucleotide. In some embodiments, the modified nucleotide comprises a locked nucleic acid (LNA). In some embodiments, the LNA is incorporated at one or both ends of the nucleotide sequence. In some embodiments, the modified nucleotide comprises a LNA chemistry modification, a peptide nucleic acid (PNA), an arabino-nucleic acid (FANA), an analogue, a derivative, or a combination thereof. In some embodiments, at least one of the one or more chemical modifications comprises a modified sugar moiety. In some embodiments, the modified sugar moiety is a 2′-O-methoxy ethyl modified sugar moiety, a 2′-methoxy modified sugar moiety, a 2′-O-alkyl modified sugar moiety, a bicyclic sugar moiety, or a combination thereof. In some embodiments, the modified sugar moiety comprises a 2′-O-methyl sugar moiety.

The method can further comprise administrating an effective amount of at least one additional therapeutic agent or at least one additional therapy to the subject for a combination therapy. In some embodiments, each of the therapeutic composition and the at least one additional therapeutic agent or therapy is administered in a separate formulation or are administered together in a single formulation. In some embodiments, the therapeutic composition and the at least one additional therapeutic agent or therapy are administered sequentially, are administered concomitantly, and/or are administered in rotation. In some embodiments, the at least one additional therapeutic agent or therapeutic therapy is selected from Idebenone, Eplerenone, VECTTOR, AVI-4658, Ataluren/PTC 124/Translama, BMN044/PR0044, CAT-1004, microDystrophin AAV gene therapy (SGT-001), Galectin-1 therapy (SB-002), LTBB4 (SB-001), rAAV2.5-CMV-minidystrophin, glutamine, NFKB inhibitors, sarcoglycan, delta (35 kDa dystrophin-associated glycoprotein), insulin like growth factor-1 (IGF-1) expression, genome editing through the CRISPR/Cas9 system, any gene delivery therapy aimed at reintroducing a functional recombinant version of the dystrophin gene, Exon skipping therapeutics, read-through strategies for nonsense mutations, cell-based therapies, utrophin upregulation, myostatin inhibition, anti-inflammatories/anti-oxidants, mechanical support devices, a biologic drug, a gene therapy or therapeutic gene modulation agent, any standard therapy for muscular dystrophy, and combinations thereof. In some embodiments, the at least one additional therapeutic agent or therapeutic therapy is selected from a percutaneous coronary intervention, coronary artery bypass grafting, thrombolytic therapy, anti-platelet therapy, heparin, warfarin, fibrinolytics, oxygen therapy, a vasodilator, pain medication, a beta blocker, an angiotensin converting enzyme (ACE) inhibitor, an angiotensin receptor blocker (ARB), a glycoprotein antagonist, a statin, an aldosterone antagonist, an implantable cardiac defibrillator (ICD), or any combination thereof.

In some embodiments, the subject is a mammal. In some embodiments, the subject is a human.

Disclosed herein include methods of preventing, inhibiting, reducing, or treating ischemic heart injury, and/or inducing cardiomyocyte regeneration, cardiac repair, vasculogenesis and/or cardiomyocyte differentiation. In some embodiments, the method comprises administering a therapeutic composition to a subject. In some embodiments, the therapeutic composition comprises a nucleotide sequence having at least 80% identity, at least 90% identity, or at least 95% identity to SEQ ID NO: 1. In some embodiments, the therapeutic composition comprises a nucleotide sequence of SEQ ID NO: 1, or a nucleotide sequence having no more than ten mismatches to the nucleotide sequence of SEQ ID NO: 1.

In some embodiments, the therapeutic composition is administrated to the subject only in a first dose and in a second dose separated from each other by a dosing interval. In some embodiments, the interval is at least 30 days.

In some embodiments, the method comprises reducing LDH levels, left ventricle (LV) cavity volume, or both in the subject. In some embodiments, the LDH levels and/or LV cavity volume is reduced by at least 20%, at least 30%, or at least 50% compared to the LDH levels and/or LV cavity volume of the subject prior to administration with the therapeutic composition. In some embodiments, the second dose of the therapeutic composition reduces CK and LDH levels, left ventricle (LV) cavity volume, size of scar, blood urea nitrogen (BUN) level, blood urea nitrogen/creatine ratios, and/or anterior wall thickness. In some embodiments, the CK and LDH levels, left ventricle (LV) cavity volume, size of scar, blood urea nitrogen (BUN) level, blood urea nitrogen/creatine ratios and/or anterior wall thickness are reduced by at least 20%, at least 30%, or at least 50% compared to the CK and LDH levels, left ventricle (LV) cavity volume, size of scar, blood urea nitrogen (BUN) level, blood urea nitrogen/creatine ratios and/or anterior wall thickness of the subject prior to administration of the second dose of the therapeutic composition. In some embodiments, the second dose of the therapeutic composition increases LV ejection fraction and LV mass, LV wall displacement/motion, albumin/globulin (A/G) ratios, percentage of lymphocytes, and/or septal and lateral walls thickness of the subject prior to administration of the second dose of the therapeutic composition. In some embodiments, the LV ejection fraction and LV mass, LV wall displacement/motion, albumin/globulin (A/G) ratios, percentage of lymphocytes, septal and/or lateral walls thickness are increased by at least 20%, at least 30%, or at least 50% compared to the LV ejection fraction and LV mass, LV wall displacement/motion, albumin/globulin (A/G) ratios, percentage of lymphocytes, septal and/or lateral walls thickness of the subject prior to administration of the second dose of the therapeutic composition.

### BRIEF DESCRIPTION OF THE DRAWINGS

FIG. 1 depicts non-limiting exemplary embodiments and data related to the animal disposition. JBT-miR2-ADD virus (N=2, #7270 and 7274) or control virus (N=3, #6668, 6669, 7278) was administered to the animals. Four animals (#7270, 7274, 6669, and 7278) survived up to 3 months, but one animal (#6668) died at 4-week.

FIG. 2 depicts non-limiting exemplary embodiments and data related to CT scan of swine hearts, illustrating how the LV mass, LV wall thickness and HU measures were taken. The measurement marked in the left panels are: 1, Ar: 101.88 mm², Av: 39.8 HU; 2, Ar: 396.46 mm², Av: 48.9 HU; 3, segment in 8.9 of 50%; 4, Ar: 420.16 mm², Av: 38.8 HU; 5, 1.9 mm; 6, 6.7 mm; 7, Ar: 351.29 mm²; 8, 6.7 mm; 9, 7.2 mm.

FIG. 3 depicts non-limiting exemplary embodiments and data related to the characteristics of the ischemia area.

FIG. 4 depicts non-limiting exemplary embodiments and data related to the ICE images of animals #7274, 6669, and 7278 at the stages of pre LAD-balloon-occlusion, at the end of 60 minutes balloon-occlusion, and 5 minutes following reperfusion/post-virus. For animal #6669, ICE image taken 5 minutes following reperfusion is shown (bottom left panel). For animals #7274 and 7278, post-virus ICE images are shown (two right panels in the bottom row).

FIG. 5A-FIG. 5F depict non-limiting exemplary embodiments and data related to the lactate dehydrogenase (LDH) and creatine kinase (CK) levels. FIG. 5A depicts the circulating CK. FIG. 5B depicts the mean CK levels at Day 80, in which placebo refers to control. FIG. 5C depicts the CK levels for Day 60 and Day 80, illustrating decrease of CK levels with treatment between Day 60 and Day 80. FIG. 5D depicts LDH levels. FIG. 5E depicts LDH levels for Day 60 and Day 80, illustrating decrease of LDH levels with treatment between Day 60 and Day 80. FIG. 5F depicts the mean LDH levels at Day 80, in which placebo refers to control.

FIG. 6A-FIG. 6Q depict non-limiting exemplary embodiments and data related to cardiac MRI images and biplane ellipsoidal model calculation of heart volumes. FIG. 6A depicts the baseline MRI images of pig 6669 (control) (diastolic volume=22.3414 ml, systole volume=10.1704 ml). FIG. 6B depicts MRI images of pig 6669 4 weeks post ischemia reperfusion injury (diastolic volume=34.1671 ml, systole volume=20.1308 ml). FIG. 6C depicts MRI images of pig 6669 8 weeks post ischemia reperfusion injury (diastolic volume=49.0010 ml, systole volume=23.1570 ml). FIG. 6D depicts MRI images of pig 6669 12 weeks post ischemia reperfusion injury (diastolic volume=30.8208 ml, systole volume=25.1788 ml). FIG. 6E depicts the baseline MRI images of pig 7270 (control) (diastolic volume=30.806607 ml, systole volume=17.602812 ml). FIG. 6F depicts MRI images of pig 7270 4 weeks post ischemia reperfusion injury (diastolic volume=57.1931 ml, systole volume=37.0812 ml). FIG. 6G depicts MRI images of pig 7270 8 weeks post ischemia reperfusion injury (diastolic volume=57.7568 ml, systole volume=40.0046 ml). FIG. 6H depicts MRI images of pig 7270 12 weeks post ischemia reperfusion injury (diastolic volume=53.3201 ml, systole volume=38.2371 ml). FIG. 6I depicts the baseline MRI images of pig 7274 (control) (diastolic volume=30.1431 ml, systole volume=13.6686 ml). FIG. 6J depicts MRI images of pig 7274 4 weeks post ischemia reperfusion injury (diastolic volume=48.3702 ml, systole volume=24.4790 ml). FIG. 6K depicts MRI images of pig 7274 8 weeks post ischemia reperfusion injury (diastolic volume=35.9389 ml, systole volume=19.3156 ml). FIG. 6L depicts MRI images of pig 7274 12 weeks post ischemia reperfusion injury (diastolic volume=53.4655 ml, systole volume=23.9569 ml). FIG. 6M depicts the baseline MRI images of pig 7278 (control) (diastolic volume=40.5127 ml, systole volume=20.3787 ml). FIG. 6N depicts MRI images of pig 7278 4 weeks post ischemia reperfusion injury (diastolic volume=46.2443 ml, systole volume=26.1051 ml). FIG. 6O depicts MRI images of pig 7278 8 weeks post ischemia reperfusion injury (diastolic volume=50.6014 ml, systole volume=26.9316 ml). FIG. 6P depicts MRI images of pig 7278 12 weeks post ischemia reperfusion injury (diastolic volume=55.5467 ml, systole volume=38.4874 ml). FIG. 6Q depicts the biplane ellipsoidal model for the calculation of LV volume.

FIG. 7A-FIG. 7B depict non-limiting exemplary embodiments and data related to delta ejection fraction percentage. FIG. 7A depicts delta ejection fraction percentage of Day 80 compared to baseline. FIG. 7B depicts delta ejection fraction percentage of Day 80 compared to Day 60. The values shown in FIG. 7A-FIG. 7B are summarized in Table 4.

FIG. 8A-FIG. 8B depict non-limiting exemplary embodiments and data related to effects of treatment on LV ejection fraction. Pig LV ejection fraction was determined by two different methods. FIG. 8A depicts determination of pig LV ejection fraction by biplane ellipsoidal. FIG. 8B depicts determination of pig LV ejection fraction by gaussian quadrature. The horizontal labels of FIG. 8A-FIG. 8B from left to right are baseline EF %, Day 30 EF %, Day 60 EF %, Day 80 EF %, Delta EF (Day 80-baseline), and Delta EF (Day 80-Day 60).

FIG. 9 depicts non-limiting exemplary embodiments and data related to the absolute ejection fraction MRI data determined by biplane ellipsoidal model.

FIG. 10A-FIG. 10B depict non-limiting exemplary embodiments and data related to delta LV mass (g). FIG. 10A depicts delta LV mass (g) of Day 80 compared to baseline. FIG. 10B depicts delta LV mass (g) of Day 80 compared to Day 60. In FIG. 10A-

FIG. 10B, placebo refers to control. The value corresponding to bars shown in FIG. 10A-FIG. 10B are summarized in Table 5.

FIG. 11 depicts non-limiting exemplary embodiments and data related to the LV mass (g) of swine determined by biplane ellipsoidal model.

FIG. 12A-FIG. 12C depict non-limiting exemplary embodiments and data related to 3D MRI LV construction and wall motion analysis. FIG. 12A depicts the end-diastolic mesh. FIG. 12B depicts percent of elements with higher SA % change. FIG. 12C depicts effects of viral gene therapy on surface area element % change, showing 300 separate elements around left ventricular cavity.

FIG. 13A-FIG. 13F depict non-limiting exemplary embodiments and data related to assessment cardiac CT data. FIG. 13A depicts LC cavity volume post-mortem CT.

FIG. 13B depicts volume of LV with low CT HU. FIG. 13C depicts percent volume of LV with low HU calculated from sum of LV mass. FIG. 13D depicts mean average HU values in low CT HU area. FIG. 13E depicts mean average HU values in normal areas of LV. FIG. 13F depicts average thickness of low CT HU areas in LV.

FIG. 14 depicts non-limiting exemplary embodiments and data related to the alkaline phosphatase (ALP) levels.

FIG. 15 depicts non-limiting exemplary embodiments and data related to the alanine transferase (ALT) levels.

FIG. 16 depicts non-limiting exemplary embodiments and data related to the aspartate aminotransferase (AST) levels.

FIG. 17 depicts non-limiting exemplary embodiments and data related to the total bilirubin levels (TBIL).

FIG. 18 depicts non-limiting exemplary embodiments and data related to direct bilirubin levels (DBIL).

FIG. 19 depicts non-limiting exemplary embodiments and data related to blood urea nitrogen (BUN) levels.

FIG. 20A-FIG. 20B depict non-limiting exemplary embodiments and data related to albumin/globulin (A/G) ratios. FIG. 20A depicts A/G ratios on Day 60. FIG. 20B depicts A/G ratios on Day 80.

FIG. 21A-FIG. 21B depict non-limiting exemplary embodiments and data related to blood urea nitrogen/creatinine (B/C) ratios. FIG. 21A depicts B/C ratios on Day 60.

FIG. 21B depicts B/C ratios on Day 80.

FIG. 22 depicts non-limiting exemplary embodiments and data related to prothrombin time (PT) levels.

FIG. 23 depicts non-limiting exemplary embodiments and data related to activated partial thromboplastin time (APTT) levels.

FIG. 24 depicts non-limiting exemplary embodiments and data related to absolute body weights of swine.

FIG. 25 depicts non-limiting exemplary embodiments and data related to log data standard curve of delta Ct values of increasing JBT-miR2-ADD levels in swine blood.

FIG. 26A-FIG. 26B depict non-limiting exemplary embodiments and data related to the targeting of JBT-miR2 to muscle and liver and inhibition of miR-99/100 and Let-7a/c. FIG. 26A depicts whole body bioluminescence imaging. The bioluminescence in FIG. 26A is scaled 0-1000 p/s/mm²/str. FIG. 26B depicts ex vivo organ viral distribution.

FIG. 27A-FIG. 27B depict non-limiting exemplary embodiments and data related to QPCR PK assay development for JBT-miR TuDS. FIG. 27A depicts standard curve delta Ct values of increasing JBT-miR2 levels in mouse serum minus blank. In FIG. 27A, x=10\hat{ }(y+0.3615)/1.574. FIG. 27B depicts that the viral genomes per 1 ml of whole blood per mouse increased with the absolute dose of JBT-miR2 injected into mice.

FIG. 28 depicts non-limiting exemplary embodiments and data related to control pig ischemic reperfusion model 3D MRI LV reconstruction.

FIG. 29 depicts non-limiting exemplary embodiments and data related to effect of JN-101 on regional wall nodal displacement (Displ/EDSA) in mice. The effect of JN-101 is comparable to that of JBT-miR2.

FIG. 30 depicts non-limiting exemplary embodiments and data related to the change of gene expression following JBT-miR2 treatment. Up regulated refers to that the expression of genes following the treatment of JBT-miR2 is greater than control virus. Down regulated refers to that the expression of genes following the treatment of JBT-miR2 is less than control virus.

FIG. 31 depicts non-limiting exemplary embodiments and data related to the enriched go terms following treatment of JBT-miR2. In FIG. 31, the columns are: 1 cellular macromolecule metabolic process, 2 macromolecule metabolic process, 3 translation, 4 cellular metabolic process, 5 nucleobase-containing compound metabolic process, 6 macromolecule biosynthetic process, 7 cellular macromolecule biosynthetic process, 8 intracellular, 9 organelle part, 10 intracellular organelle part, 11 ribosome, 12 intracellular organelle, 13 macromolecular complex, 14 intracellular part, 15 structural constituent of ribosome, 16 nucleic acid binding.

FIG. 32A-FIG. 32B depict non-limiting exemplary embodiments and data related to regulation of gene expression. FIG. 32A depicts heatmap showing changes in gene expression. FIG. 32B depicts the statistics of pathway enrichment.

### DETAILED DESCRIPTION

In the following detailed description, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods of preventing, inhibiting, reducing, or treating ischemic heart injury, and/or inducing cardiomyocyte regeneration, cardiac repair, vasculogenesis and/or cardiomyocyte differentiation. In some embodiments, the method comprises administering a therapeutic composition to a subject. The therapeutic composition can comprise a nucleotide sequence having at least 80%, at least 90%, or at least 95% identity to SEQ ID NO: 1.

Disclosed herein include methods of preventing, inhibiting, reducing, or treating ischemic heart injury. The method comprises administering a therapeutic composition to a subject before, during, and/or after a cardiac ischemic event. The therapeutic composition is administrated in a first dose and a second dose separated by a dosing interval.

Disclosed herein include methods of inducing cardiomyocyte regeneration, cardiac repair, vasculogenesis and/or cardiomyocyte differentiation. The method comprises administering a therapeutic composition to a subject. The therapeutic composition is administrated in a first dose and a second dose separated by a dosing interval.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “about”, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The term “pharmaceutical formulation”, as used herein, refers to a composition suitable for administering to an individual that includes a pharmaceutical agent.

As used herein, the term “ischemia-reperfusion injury” (IRI), shall be given its ordinary meaning, and shall also refer to tissue damage (e.g., injury) caused by ischemia, reperfusion, or ischemia followed by reperfusion. Thus, the term “ischemia-reperfusion injury” includes injuries caused by ischemia, reperfusion injuries, and injuries caused by ischemia followed by reperfusion. Myocardial infarction (MI) is a type of cardiac ischemia event that can result in IR injury of the heart tissues. As used herein, the “injury resulting from ischemia,” “injury caused by ischemia” and “ischemic injury” can refer to an injury to a cell, tissue or organ caused by ischemia, or an insufficient supply of blood (e.g., due to a blocked artery), and, thus, oxygen, resulting in damage or dysfunction of the tissue or organ. In some embodiments, the term “ischemia-reperfusion injury” refers to an injury resulting from the restoration of blood flow to an area of a tissue or organ that had previously experienced deficient blood flow due to an ischemic event.

As used herein, “pharmaceutically acceptable” carriers, excipients, diluents, adjuvants, or stabilizers can refer to the ones nontoxic to the cell or subject being exposed thereto (preferably inert) at the dosages and concentrations employed or that have an acceptable level of toxicity as determined by the skilled practitioner.

As used herein, “stuffer” or “stuffer sequence” can refer to a heterologous polynucleotide sequence inserted within a vector, which resizes or adjusts the total length of the vector. For example, a stuffer sequence can be inserted between or outside AAV inverted terminal repeat (ITR) sequences, so that the length of a viral genome is adjusted to near or at the normal size of the viral genomic sequence that is packaged or encapsidated to form infectious viral particles. The stuffer sequence is inserted in a manner that it does not interfere with expression of genes in the vector.

As used herein, “administering” can refer to providing a pharmaceutical agent or composition to a subject, and includes, but is not limited to, administering by a medical professional and self-administering.

As used herein, “parenteral administration” can refer to administration through injection or infusion. Parenteral administration includes, but is not limited to, subcutaneous administration, intravenous administration, intramuscular administration, intra-arterial administration, and intracranial administration. “Subcutaneous administration” means administration just below the skin. “Intravenous administration” means administration into a vein. “Intraarterial administration” means administration into an artery.

As used herein, a “viral vector” is a viral-derived nucleic acid molecule that is capable of transporting another nucleic acid into a cell.

As used herein, “amino acid” can refer to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and 0-phospho serine. Amino acid analogs can refer to compounds that have the same basic chemical structure as a naturally occurring amino acid, e.g., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids can be referred to herein by either their commonly known three letter symbols or by the one-letter symbols recommended by the IUPAC-IUB Biochemical Nomenclature Commission. Nucleotides, likewise, may be referred to by their commonly accepted single-letter codes. “Antisense compound” means a compound having a nucleobase sequence that will allow hybridization to a target nucleic acid. In certain embodiments, an antisense compound is an oligonucleotide having a nucleobase sequence complementary to a target nucleic acid.

As used herein, the terms “complementary” or “complementarity” can refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing. The terms “protein,” “peptide,” and “polypeptide” are used interchangeably to denote an amino acid polymer or a set of two or more interacting or bound amino acid polymers. These terms, as used herein, encompass amino acid polymers in which one or more amino acid residue is an artificial chemical mimetic of a corresponding naturally occurring amino acid, as well as to naturally occurring amino acid polymers and non-naturally occurring amino acid polymer.

As used herein, “expression” refers to the process of converting genetic information of a polynucleotide into RNA through transcription, which is typically catalyzed by an enzyme, RNA polymerase, and, where the RNA encodes a polypeptide, into protein, through translation of mRNA on ribosomes to produce the encoded protein. The term “expression cassette” as used herein, refers to a nucleic acid construct that encodes a gene, a protein, or a functional RNA operably linked to expression control elements, such as a promoter, and optionally, any or a combination of other nucleic acid sequences that affect the transcription or translation of the gene, such as, but not limited to, a transcriptional terminator, a ribosome binding site, a splice site or splicing recognition sequence, an intron, an enhancer, a polyadenylation signal, an internal ribosome entry site, etc.

As used herein, the term “vector” refers to a nucleic acid construct, typically a plasmid or a virus, used to transmit genetic material to a host cell. Vectors can be, for example, viruses, plasmids, cosmids, or phage. A vector as used herein can be composed of either DNA or RNA. The term “vector” includes cloning vectors and expression vectors, as well as viral vectors and integrating vectors. An “expression vector” is a vector that is capable of directing the expression of a gene, or protein encoded by one or more genes carried by the vector when it is present in the appropriate environment.

As used herein, “conservatively modified variants” applies to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical nucleotide sequences. Because of the degeneracy of the genetic code, a large number of functionally identical nucleic acids can encode any given protein. For instance, the codons GCA, GCC, GCG and GCU all encode the amino acid alanine. Thus, at every position where an alanine is specified by a codon, the codon can be altered to any of the corresponding codons described above without altering the encoded polypeptide. Such nucleic acid variations are “silent variations,” which are one species of conservatively modified variations. Any one of the nucleic acid sequences described herein which encodes a polypeptide also describes every possible silent variation of the nucleic acid. One of ordinary skill in the art will recognize that each codon in a nucleic acid (except AUG, which is ordinarily the only codon for methionine, and TGG, which is ordinarily the only codon for tryptophan) can be modified to yield a functionally identical molecule. Accordingly, all silent variations of a nucleic acid which encodes a polypeptide are implicit in each of the described sequences with respect to its expression product, but not with respect to actual probe sequences. In addition or alternatively, a variant can comprises deletions, substitutions, additions of one or more nucleotides at the 5′ end, 3′ end, and/or one or more internal sites in comparison to the reference polynucleotide. Similarities and/or differences in sequences between variants and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a particular polynucleotide disclosed herein, including, but not limited to, a miRNA, will have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisan.

As used herein, “identical” or “percent identity,” in the context of two or more nucleic acids or proteins, can refer to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a test sequence. This definition also includes sequences that have deletions and/or additions, as well as those that have substitutions. Sequence identity typically exists over a region that is at least about 50 amino acids or nucleotides in length, or over a region that is 50-100 amino acids or nucleotides in length, or over the entire length of a given sequence.

As used herein, the term “construct” can refer to any recombinant nucleic acid molecule such as an expression cassette, plasmid, cosmid, virus, autonomously replicating polynucleotide molecule, phage, or linear or circular, single-stranded or double-stranded, DNA or RNA polynucleotide molecule, derived from any source, capable of genomic integration or autonomous replication, comprising a nucleic acid molecule where one or more nucleic acid sequences has been linked in a functionally operative manner, e.g. operably linked.

As used herein, “transfection” or “transfecting” can refer to a process of introducing a nucleic acid molecule to a cell using non-viral or viral-based methods. The nucleic acid molecule can be a sequence encoding complete proteins or functional portions thereof. Typically, a nucleic acid vector comprises the elements necessary for protein expression (e.g., a promoter, transcription start site, etc.). Non-viral methods of transfection include any appropriate transfection method that does not use viral DNA or viral particles as a delivery system to introduce the nucleic acid molecule into the cell. Exemplary non-viral transfection methods include, but are not limited to, calcium phosphate transfection, liposomal transfection, nucleofection, sonoporation, transfection through heat shock, magnetifection, and electroporation. For viral-based methods, any one of useful viral vectors known in the art can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures known in the art.

As used herein, the term “heterologous,” when used with reference to portions of a nucleic acid or protein, can refer to that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

As used herein, the term “gene” is used broadly to refer to any segment of nucleic acid molecule that encodes a protein or that can be transcribed into a functional RNA. Genes may include sequences that are transcribed but are not part of a final, mature, and/or functional RNA transcript, and genes that encode proteins may further comprise sequences that are transcribed but not translated, for example, 5′ untranslated regions (5′-UTR), 3′ untranslated regions (3′-UTR), introns, etc. Further, genes may optionally further comprise regulatory sequences required for their expression, and such sequences may be, for example, sequences that are not transcribed or translated. Genes can be obtained from a variety of sources, including cloning from a source of interest or synthesizing from known or predicted sequence information, and may include sequences designed to have desired parameters.

As used herein, “internucleoside linkage” can refer to a covalent linkage between adjacent nucleosides.

As used herein, “nucleobase” can refer to a heterocyclic moiety capable of non-covalently pairing with another nucleobase.

As used herein, “nucleoside” can refer to a nucleobase linked to a sugar. “Linked nucleosides” means nucleosides joined by a covalent linkage. “Nucleotide” means a nucleoside having a phosphate group covalently linked to the sugar portion of a nucleoside.

As used herein, “miR antagonist” can refer to an agent designed to interfere with or inhibit the activity of an miRNA. In certain embodiments, an miR antagonist comprises an antisense compound targeted to an miRNA. In certain embodiments, an miR antagonist comprises a modified oligonucleotide having a nucleobase sequence that is complementary to the nucleobase sequence of an miRNA, or a precursor thereof. In certain embodiments, an miR antagonist comprises a small molecule, or the like that interferes with or inhibits the activity of an miRNA.

As used herein, “miR-9a-5p antagonist” can refer to an agent designed to interfere with or inhibit the activity of miR-9a-5p. “miR-100-5p antagonist” can refer to an agent designed to interfere with or inhibit the activity of miR-100-5p. “Let-7a-5p antagonist” can refer to an agent designed to interfere with or inhibit the activity of Let-7a-5p. “Let-7c-5p antagonist” can refer to an agent designed to interfere with or inhibit the activity of Let-7c-5p.

As used herein, “modified oligonucleotide” can refer to an oligonucleotide having one or more chemical modifications relative to a naturally occurring terminus, sugar, nucleobase, and/or internucleoside linkage.

As used herein, “modified internucleoside linkage” can refer to any change from a naturally occurring internucleoside linkage.

As used herein, “phosphorothioate internucleoside linkage” can refer to a linkage between nucleosides where one of the non-bridging atoms is a sulfur atom.

As used herein, “modified sugar” can refer to substitution and/or any change from a natural sugar.

As used herein, “modified nucleobase” can refer to any substitution and/or change from a natural nucleobase.

As used herein, “5-methylcytosine” can refer to a cytosine modified with a methyl group attached to the 5′ position.

As used herein, “2′-O-methyl sugar” or “2′-OMe sugar” can refer to a sugar having an O-methyl modification at the 2′ position.

As used herein, “2′-O-methoxyethyl sugar,” “2′-MOE sugar,” or “2′-O-methyl sugar” can refer to a sugar having an O-methoxyethyl modification at the 2′ position.

As used herein, “2′-O-fluoro sugar,” “2′-F sugar,” or “2′-O-methyl sugar” can refer to a sugar having a fluoro modification of the 2′ position.

As used herein, “bicyclic sugar moiety” can refer to a sugar modified by the bridging of two non-geminal ring atoms.

As used herein, “2′-O-methoxyethyl nucleoside” can refer to a 2′-modified nucleoside having a 2′-O-methoxyethyl sugar modification.

As used herein, “2′-fluoro nucleoside” can refer to a 2′-modified nucleoside having a 2′-fluoro sugar modification.

As used herein, “2′-O-methyl” nucleoside can refer to a 2′-modified nucleoside having a 2′-O-methyl sugar modification.

As used herein, “bicyclic nucleoside” can refer to a 2′-modified nucleoside having a bicyclic sugar moiety.

As used herein, the terms “miR,” “mir,” and “miRNA” are used interchangeably and to refer to microRNA, a class of small RNA molecules that are capable of hybridizing to and regulating the expression of a coding RNA. In certain embodiments, a miRNA is the product of cleavage of a pre-miRNA by the enzyme Dicer. These terms as provided herein refer to a nucleic acid that forms a double-stranded RNA which has the ability to reduce or inhibit expression of a gene or target gene when expressed in the same cell as the gene or target gene. The complementary portions of the nucleic acid that hybridize to form the double-stranded molecule typically have substantial or complete identity. In some embodiments, a “microRNA” refers to a nucleic acid that has substantial or complete identity to a target gene and forms a double-stranded miRNA. In some embodiments, the miRNA of the disclosure inhibits gene expression by interacting with a complementary cellular mRNA thereby interfering with the expression of the complementary mRNA. In some embodiments, the double-stranded miRNA of the present disclosure is at least about 15-50 nucleotides in length (e.g., each complementary sequence of the double-stranded miRNA is 15-50 nucleotides in length, and the double-stranded miRNA is about 15-50 base pairs in length). In some embodiments, the length is 20-30 base nucleotides, preferably about 20-25 or about 24-29 nucleotides in length, e.g., 20, 21, 22, 23, 24, 25, 26, 27, 28, 29, or 30 nucleotides in length. In some embodiments of the disclosure, the microRNA is selected from, or substantially similar to a microRNA selected from miR-9a-5p, miR-100-5p, Let-7a-5p, and Let-7c-5p.

As used herein, the term “anti-miRNA” is used interchangeably with the term “anti-miR”, which can refer to an oligonucleotide capable of interfering with or inhibiting one or more activities of one or more target microRNAs. In some embodiments, the anti-miRNA is a chemically synthesized oligonucleotide. In some embodiments, the anti-miRNA is a small molecule. In some embodiments, the anti-miRNA is an miR antisense molecule. “Seed region” means nucleotides 2 to 6 or 2 to 7 from the 5′-end of a mature miRNA sequence.

As used herein, “miRNA precursor” can refer to a transcript that originates from a genomic DNA and that comprises a non-coding, structured RNA comprising one or more miRNA sequences. For example, in certain embodiments a miRNA precursor is a pre-miRNA. In certain embodiments, a miRNA precursor is a pri-miRNA.

As used herein, “pre-miRNA” or “pre-miR” can refer to a non-coding RNA having a hairpin structure, which contains a miRNA. In certain embodiments, a pre-miRNA is the product of cleavage of a pri-miR by the double-stranded RNA-specific ribonuclease known as Drosha. Without wishing to be bound by any particular theory, it is believed that in the cytoplasm, the pre-miRNA hairpin is cleaved by the RNase III enzyme Dicer. This endoribonuclease interacts with 5′ and 3′ ends of the hairpin and cuts away the loop joining the 3′ and 5′ arms, yielding an imperfect miRNA:miRNA duplex of about 22 nucleotides in length. Although either strand of the duplex may potentially act as a functional miRNA, it is believed that only one strand is usually incorporated into the RNA-induced silencing complex (RISC) where the miRNA and its mRNA target interact. The remaining strand (sense strand) is degraded. The RNA-induced silencing complex, or RISC, is a multiprotein complex, specifically a ribonucleoprotein, which incorporates one strand of a single-tran ed RNA (ssRNA) fragment, such as microRNA (miRNA), or double-stranded small interfering RNA (siRNA). “Modulation” means to a perturbation of function or activity. In certain embodiments, modulation means an increase in gene expression. In certain embodiments, modulation means a decrease in gene expression. The term “microRNA modulator” as used herein can refer to an agent capable of modulating the level of expression of a microRNA (e.g., let-7a, let-7c, miR-100, miR-99). In some embodiments, the microRNA modulator is encoded by a nucleic acid. In some embodiments, the microRNA modulator is a small molecule (e.g., a chemical compound or synthetic microRNA molecule). In some embodiments, the microRNA modulator decreases the level of expression of a microRNA compared to the level of expression in the absence of the microRNA modulator. Where the microRNA modulator decreases the level of expression of a microRNA relative to the absence of the modulator, the microRNA modulator is an antagonist of the microRNA. In some embodiments, the microRNA modulator increases the level expression of a microRNA compared to the level of expression in the absence of the microRNA modulator. Where the microRNA modulator increases the level of expression of a microRNA relative to the absence of the modulator, the microRNA modulator is an agonist of the microRNA.

As used herein, the term “myocardial cell” includes any cell that is obtained from, or present in, myocardium such as a human myocardium and/or any cell that is associated, physically and/or functionally, with myocardium. In some embodiments disclosed herein, a myocardial cell is a cardiomyocyte.

As used herein, “nucleotide” can refer to naturally occurring nucleotides as well as non-naturally occurring nucleotides. Thus, “nucleotides” includes not only the known purine and pyrimidine heterocycles-containing molecules, but also heterocyclic analogues and tautomers thereof. Non-limiting examples of other types of nucleotides are molecules containing adenine, guanine, thymine, cytosine, uracil, purine, xanthine, diaminopurine, 8-oxo-N6-methyladenine, 7-deazaxanthine, 7-deazaguanine, N4,N4-ethanocytosin, N6,N6-ethano-2,6-diaminopurine, 5-methylcytosine, 5-(C3-C6)-alkynylcytosine, 5-fluorouracil, 5-bromouracil, pseudoisocytosine, 2-hydroxy-5-methyl-4-triazolopyridin, isocytosine, isoguanine, inosine and the “non-naturally occurring” nucleotides described in U.S. Pat. No. 5,432,272. The term “nucleotide” is intended to cover every and all of these examples as well as analogues and tautomers thereof.

The term “operably linked”, as used herein, denotes a functional linkage between two or more sequences. For example, an operably linkage between a polynucleotide of interest and a regulatory sequence (for example, a promoter) is functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non-contiguous.

The terms “promoter,” “promoter region,” or “promoter sequence,” as used interchangeably herein, can refer to a nucleic acid sequence capable of binding RNA polymerase to initiate transcription of a gene in a 5′ to 3′ (“downstream”) direction. The specific sequence of the promoter typically determines the strength of the promoter. For example, a strong promoter leads to a high rate of transcription initiation. A gene is “under the control of” or “regulated by” a promoter when the binding of RNA polymerase to the promoter is the proximate cause of said gene's transcription. The promoter or promoter region typically provides a recognition site for RNA polymerase and other factors necessary for proper initiation of transcription. A promoter may be isolated from the 5′ untranslated region (5′ UTR) of a genomic copy of a gene. Alternatively, a promoter may be synthetically produced or designed by altering known DNA elements. Also considered are chimeric promoters that combine sequences of one promoter with sequences of another promoter. A promoter can be used as a regulatory element for modulating expression of an operably linked polynucleotide molecule such as, for example, a coding sequence of a polypeptide or a functional RNA sequence. Promoters may contain, in addition to sequences recognized by

RNA polymerase and, preferably, other transcription factors, regulatory sequence elements such as cis-elements or enhancer domains that affect the transcription of operably linked genes. In some embodiments, a promoter can be “constitutive.” In some embodiments, a promoter may be regulated in a “tissue-specific” or “tissue-preferred” manner, such that it is only active in transcribing the operable linked coding region in a specific tissue type or types. In some embodiments, for therapeutic purposes, the promoter can be a tissue-specific promoter, which supports transcription in cardiac and skeletal muscle cell. Further information in this regard can be found in, for example,

PCT Patent Publication WO2004041177A2, which is hereby incorporated by reference in its entirety. In some embodiments, a promoter may comprise “naturally-occurring” or “synthetically” assembled nucleic acid sequences.

Expression of a transfected gene can occur transiently or stably in a host cell. During “transient expression” the transfected nucleic acid is not integrated into the host cell genome, and is not transferred to the daughter cell during cell division. Since its expression is restricted to the transfected cell, expression of the gene can be lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as in subsequent excision.

As used herein, “inhibitor,” “repressor” or “antagonist” or “downregulator”, as used interchangeably, can refer to a substance, agent, or molecule that results in a detectably lower expression or activity level of a target gene as compared to a control. The inhibited expression or activity can be 10%, 20%, 30%, 40%, 50%, 60%, 70%, 80%, 90% or less than that in a control. In some embodiments, the inhibition is 1.5-fold, 2-fold, 3-fold, 4-fold, 5-fold, 10-fold, or more in comparison to a control. In some embodiments, an antagonist is an anti-miR.

As used herein, “treatment” can refer to a clinical intervention made in response to a disease, disorder or physiological condition manifested by a patient or to which a patient may be susceptible. The aim of treatment includes, but is not limited to, the alleviation or prevention of symptoms, slowing or stopping the progression or worsening of a disease, disorder, or condition and/or the remission of the disease, disorder or condition. “Treatments” can refer to one or both of therapeutic treatment and prophylactic or preventative measures. Subjects in need of treatment include those already affected by a disease or disorder or undesired physiological condition as well as those in which the disease or disorder or undesired physiological condition is to be prevented. In some embodiments of the disclosure, the terms “treatment,” “therapy,” and “amelioration” can refer to any reduction in the severity of symptoms, e.g., of a neurodegenerative disorder or neuronal injury.

As used herein, the terms “treat” and “prevent” are not intended to be absolute terms. Treatment can refer to any delay in onset, amelioration of symptoms, and improvement in patient survival, increase in survival time or rate, etc., or a combination thereof. The effect of treatment can be compared to an individual or pool of individuals not receiving the treatment, or to the same patient prior to treatment or at a different time during treatment. In some embodiments, the severity of disease or disorder in an individual can be reduced by at least 10%, as compared, e.g., to the individual before administration or to a control individual not undergoing treatment. In some embodiments, the severity of disease or disorder in an individual is reduced by at least 25%, 50%, 75%, 80%, or 90%, or in some embodiments, no longer detectable using standard diagnostic techniques.

As used herein, the term “effective amount” or “therapeutically effective amount” refers to an amount sufficient to effect beneficial or desirable biological and/or clinical results. In some embodiments, the term refers to that amount of the therapeutic agent sufficient to ameliorate a given disorder or symptoms. For example, for the given parameter, a therapeutically effective amount can show an increase or decrease of at least 5%, 10%, 15%, 20%, 25%, 40%, 50%, 60%, 75%, 80%, 90%, or at least 100% compared to a control. Therapeutic efficacy can also be expressed as “-fold” increase or decrease. For example, a therapeutically effective amount can have at least a 1.2-fold, 1.5-fold, 2-fold, 5-fold, or more effect over a control.

The terms “subject,” “patient,” “individual in need of treatment” and like terms are used interchangeably and refer to, except where indicated, a mammal subject that is the object of treatment, observation, or experiment. As used herein, “mammal” refers to a subject belonging to the class Mammalia and includes, but not limited to, humans, domestic and farm animals, zoo animals, sports and pet animals. Non-limiting examples of mammals include humans, and non-human primates, mice, rats, sheep, dogs, horses, cats, cows, goats, pigs, and other mammalian species. In some embodiments, the mammal is a human. However, in some embodiments, the mammal is not a human. The term does not necessarily indicate that the subject has been diagnosed with a particular disease or disorder, but typically refers to a subject under medical supervision. “Subject suspected of having” means a subject exhibiting one or more clinical indicators of a disease or condition. In certain embodiments, the disease or condition is a muscular dystrophy (MD) disorder.

As used herein, “target nucleic acid,” “target RNA,” “target RNA transcript” and “nucleic acid target” can refer to a nucleic acid capable of being targeted by antagonists. “Targeting” can refer to the process of design and selection of nucleobase sequence that will hybridize to a target nucleic acid and induce a desired effect. “Targeted to” can refer to having a nucleobase sequence that will allow hybridization to a target nucleic acid to induce a desired effect. In certain embodiments, a desired effect is reduction of a target nucleic acid.

As used herein, the term “variant” can refer to a polynucleotide (or polypeptide) having a sequence substantially similar to a reference polynucleotide (or polypeptide). In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by one of ordinary skill in the art.

Disclosed herein includes that 1) two single doses of JBT-miR2-ADD that delivers inhibitors to miR-99, miR-100, let-7a and let-7c administered intracoronary and intravenous (IV) respectively can re-activate an underlying cardiac regeneration process in hearts with IR injury. In some embodiments, the heart is a heart of the subject (e.g., pig, or a primate (e.g., human)). 2) Repeat dosing of JBT-miR2-ADD is safe, with no liver toxicity or arrhythmia. In some embodiments, the lack of liver toxicity or arrhythmia was noted up to 10-wks post-ischemic reperfusion (IR). 3) JBT-miR2-ADD reduces scar in the LV. In some embodiments, a composition comprising the miRs disclosed herein (e.g., via JBT-miR2-ADD) can be administered to the subject by intracoronary injection at reperfusion, e.g., using a catheter. In some embodiments, a composition comprising the miRs disclosed herein (e.g., via JBT-miR2-ADD) can be administered by intravenous injection one month, two months, three months, or a number or a range between any these two numbers, after Ischemia. In some embodiments, the second dose of a composition comprising the miRs disclosed herein (e.g., via JBT-miR2-ADD) can be administered by intravenous injection one month, two months, three months, or a number or a range between any these two numbers, after Ischemia. As disclosed herein, the composition comprising the miRs can be used to increase heart function when administered to the patient immediately after a heart attack; and/or 1, 2, 3 or 4 months (or a number or a range between any two of these values) after a heart attack to the patient with established Ischemic heart disease.

In some embodiment, an adenoviral delivery therapeutic to regenerate heart muscle after a surgically induced myocardial infarction in a subject is provided. In some embodiments, the subject is human, a primate or swine. In some embodiments, the swine is female 4 to 6-month-old, Yucatan pig. In some embodiments, the therapeutic is JBT-miR2-ADD. JBT-miR2-ADD is a single AAV2/9 virus that expresses inhibitors to four microRNAs (miRs: Let-7a/c, miR-99 and miR-100) (SEQ ID NO. 1). In some embodiments, JBT-miR2-ADD was delivered by IC infusion at the time or reperfusion, after a 60-minute balloon occlusion between the first and second diagonal branch of the left anterior descending coronary artery (LAD). The subjects (e.g., swine, a primate, human) were then dosed a second time with an IV bolus injection.

JBT-miR2-ADD (SEQ ID NO: 1)
ITR-pAV-U6-Plasmid sequence-U6 Promoter - miR-99a - spacer - let-7a/c -
H1 Promoter-U06 - stuffer - pAV-U6 Plasmid spacer-ITR 5′
(SEQ ID NO: 1)
CCACTCCCTCTATGCGCGCTCGCTCACTCACTCGGCCCTGGAGACCAAAGGTCTCCAGACTGCCGGCCTC
TGGCCGGCAGGGCCGAGTGAGTGAGCGAGCGCGCATAGAGGGAGTGGGTACCTCCATCATCTAGGTTTGC
CCCTGCAGGCAGCTGCGCGCTCGCTCGCTCACTGAGGCCGCCCGGGCGTCGGGCGACCTTTGGTCGCCCG
GCCTCAGTGAGCGAGCGCGCAGAGAGGGAGTGGCCAACTCCATCACTAGGGGTTCCTGCGGCCGCACGCG
TCTAGTTATTAATAGTAATCGAATTCGTGTTACTCATAACTAGTAAGGTCGGGCAGGAAGAGGGCCTATT
TCCCATGATTCCTTCATATTTGCATATACGATACAAGGCTGTTAGAGAGATAATTAGAATTAATTTGACT
GTAAACACAAAGATATTAGTACAAAATACGTGACGTAGAAAGTAATAATTTCTTGGGTAGTTTGCAGTTT
TAAAATTATGTTTTAAAATGGACTATCATATGCTTACCGTAACTTGAAAGTATTTCGATTTCTTGGGTTT
ATATATCTTGTGGAAAGGACTGTAAACACAAAGATATTAGTACAAAATACGTGACGTAGAAAGTAATAAT
TTCTTGGGTAGTTTGCAGTTTTAAAATTATGTTTTAAAATGGACTATCATATGCTTACCGTAACTTGAAA
GTATTTCGATTTCTTGGGTTTATATATCTTGTGGAAAGGACGCGAACACCGACGGCGCTAGGATCATCTT
GTACCCGTAGACATTTCCGATCTTGTGGTTGTATTCTGTGACCAGAATACTTGTACCCGTAGACATTTCC
GATCTTGTGGTTGATGATCCTAGCGCCGTCTTTTTTGAGCTCAAAAAAGACGGCGCTAGGATCATCAACA
ACTATACAACCAATGTACTACCTCACAAGTATTCTGGTCACAGAATACAACAACTATACAACCAATGTAC
TACCTCACAAGATGATCCTAGCGCCGTCGGATCCGAGTGGTCTCATACAGAACTTATAAGATTCCCAAAT
CCAAAGACATTTCACGTTTATGGTGATTTCCCAGAACACATAGCGACATGCAAATATTGCAGGGCGCCAC
TCCCCTGTCCCTCACAGCCATCTTCCTGCCAGGGCGCACGCGCGCTGGGTGTTCCCGCCTAGTGACACTG
GGCCCGCGATTCCTTGGAGCGGGTTGATGACGTCAGCGTTCACTAGTTATGAGTAACACGAATTCGATTA
CTATTAATAACTAGACGCGTGCGGCCGCAGGAACCCCTAGTGATGGAGTTGGCCACTCCCTCTCTGCGCG
CTCGCTCACTGAGGCCGGGCGACCAAAGGTCGCCCGACGCCCGGGCGGCCTCAGTGAGCGAGCGAGCGCG
CAGCTGCCTGCAGGACATGTGAGCAAAAGGCCAGCcgtttagtgaaccgtcagatcgcctggagacgcca
tccacgctgttttgacctccatagaagacaccgggaccgatccagcctccggactctagagagacgtaca
aaaaagagcaagaagctaaaaaagatttaaaaattatttttagcgcagttaatggaacaggaactaaatt
taccccaaaaatattacgtgaatcaggatataacgttattgaggttgaagagcatgcatttgaagatgaa
acatttaaaaatgttgtaaatccaaatccagaatttgatcctgcatgaaaaataccgcttgaatatggta
ttaaacatgatgcagatattattattatgaatgacccagatgctgacagatttggaatggcaataaaaca
tgatggtcattttgtaagattagatggaaatcaaacaggaccaattttaattgattgaaaattatcaaat
ctaaaacgcttaaatagcattccaaaaaatccggctctatattcaagttttgtaacaagtgatttgggtg
atagaatcgctcatgaaaaatatggagttaatattgtaaaaactttaactggatttaaatgaatgggtag
agaaattgctaaagaagaagataacggattaaattttgtttttgcttatgaagaaagttatggatatgta
attgatgactcagctagagataaagatggaatacaagcttctatattaatagcagaggctgcttgatttt
ataaaaaaaaaataaaacattagtagactatttagaagatttatttaaagaaatgggtgcatattacac
tttcactttaaacttgaattttaaaccagaagaaaagaaattaaaaattgaaccattaatgaaatcattg
agagcaacacccttaactcaaattgctggacttaaagttgttaatgttgaagactacatcgatggaatgt
ataatatgccaggacaagacttactaaaattttatttagaagataagtcatgatttgctgttcgcccaag
tggaactgaacctaaactaaaaatttattttataggtgttggtgaatctgttcaaaacgctaaagttaaa
gtagacgaaattattaaagaattaaaattaaaaatgaatatataggagaaaaaatgaaactaaacaaata
tatagatcacacattattaaaacaagatgctacgaaagctgaaattaaacaattatgtgatgaagcaatt
gaatttgattttgcaacagtttgtgttaattcatattgaacaagctattgtaaagaattattaaaaggca
caaatgtaggaataacaaatgttgtaggttttcctctaggtgcatgcacaacagctacaaaagcattcga
agtttctgaagcaattaaagatggtgcaacagaaattgatatggtattaaatattggtgcattaaaagac
aaaaattatgaattagttttagaagacatgaaagctgtaaaaaaagcagctggatcacatgttgttaaat
gtattatggaaaattgtttattaacaaaagaagaaatcatgaaagcttgtgaaatagctgttgaagctgg
attagaatttgttaaaacatcaacaggattttcaaaatcaggtgcaacatttgaagatgttaaactaatg
aagtcagttgttaaagacaatgctttagttaaagcagctggtggagttagaacatttgaagatgctcaaa
aaatgattgaagcaggagctgaccgcttaggaacaagtggtggagtagctattattaaaggtgaagaaaa
caacgcgagttactaaaactagcgtttttttattttgctcatttttattaaaagtttgcaaaaaggaaca
taaaaattctaattattgatactaaagttattaaaaagaagattttggttgattttataaaggtcataga
atataatattttagcatgtgtattttgtgtgctcatttacaaccgtctcgcggccgcgggACGCGGTAAC
CACGTGCGGACCGAGCGGCCGCAGGAACCCCTAGTGATGGAGTTGGCCACTCCCTCTCTGCGCGCTCGCT
CGCTCACTGAGGCCGGGCGACCAAAGGTCGCCCGACGCCCGGGCTTTGCCCGGGCGGCCTCAGTGAGCGA
GCGAGCGCGCAGCTGCCTGCAGG

Cardiac Diseases and Micro-Ribonucleic Acid (miRNA)

Cardiac disease or heart disease is a disease for which several classes or types exist (e.g., ischemic heart disease (IHD), dilated cardiomyopathy (DCM), aortic stenosis (AS)) and many require unique treatment strategies. Thus, heart disease is not a single disease, but rather a family of disorders arising from distinct cell types (e.g., myocardial cells) by distinct pathogenetic mechanisms. The challenge of heart disease treatment has been to target specific therapies to particular heart disease types, to maximize effectiveness and to minimize toxicity. As used herein, cardiac disease encompasses the following non-limiting examples: heart failure (e.g., congestive heart failure), IHD, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis, AS, aortic regurgitation, mitral stenosis, mitral regurgitation, mitral prolapse, pulmonary stenosis, pulmonary regurgitation, tricuspid stenosis, tricuspid regurgitation, congenital disorder, genetic disorder, or a combination thereof.

Ischemic heart disease (IHD) is the single largest cause of death in the world and is commonly a consequence of acute myocardial infarction (MI). A heart attack or MI results from limitation of coronary blood flow to the heart, causing ischemia and ultimately irreversible death of cardiomyocytes. With the number of adults aged 65 years and older projected to increase an additional 44% from 2017 to 2030, innovative and effective approaches to prevent and treat IHD, particularly the substantially increasing rates of heart failure (HF), are needed. The increase of morbidity and mortality from IHD can be preponderantly attributed to the lack of therapies to replace the lost cardiac muscle cells incurred during a MI and to prevent the transition to HF. The size of a myocardial infarct correlates with the degree of deterioration of heart function, compromise of contractile reserve, and the overtime likelihood of mortality from heart failure (HF).

Prompt restoration of arterial perfusion with thrombolytic and antiplatelet therapy during percutaneous coronary intervention has led to a decline in acute mortality from MI. However, the prevalence of HF among survivors has augmented, because irreversible cardiomyocyte death results in residual inducible ischemia and permanent scarring. A major pathologic problem is the failure of human adult cardiomyocytes to regenerate themselves endogenously following an MI. This is compounded by a lack of adjunctive treatments, pharmacologic or cellular, that can be administered in conjunction with reperfusion, or after to stimulate regeneration of heart muscle. Effective promotion of endogenous cardiomyocyte regeneration in the ischemic heart with concomitant reduction of scar size can offer a powerful new treatment for MI and its adverse pathophysiologic consequences.

In 2001, mitosis in cardiomyocytes was evident after a myocardial infarction. Studies by others confirmed that adult mammalian hearts can elicit a primitive regeneration response upon injury with mature differentiated mononuclear mammalian cardiomyocytes re-entering the cell cycle upon application of chemical compounds that target specific signaling pathways.

miRNAs (miRs) are small non-coding RNA molecules conserved in plants, animals, and some viruses, which function in RNA silencing and post-transcriptional regulation of gene expression. Identified in 1993, they are a vital and evolutionarily component of genetic regulation. They function via base-pairing and silencing complementary sequences within mRNA molecules thereby modulating target protein expression and downstream signaling pathways. There are 1000 known miRs in the human genome that can target 60% of human genes. In animals, miRNAs are processed from larger primary transcripts (pri-miRNA or pri-miR) through an approximate 60-bp hairpin precursor (pre-miRNA or pre-miR) into the mature forms (miRNA) by two RNAse III enzymes Drosha and Dicer. The mature miRNA is loaded into the 50 ribonucleoprotein complexes (RISC), where it typically guides the downregulation of target mRNA through base pair inter- actions. Pri-miRNAs are transcribed by RNA polymerase II and predicted to be regulated by transcription factors in an inducible manner. While some miRNAs show ubiquitous expression, others exhibit only limited developmental stage-, tissue- or cell type-specific patterns of expression.

miRNAs play a regulatory role in myocardial growth, fibrosis, and remodeling in myocardial tissue. In particular, ribonucleic acid interference (RNAi) technology is an area of intense research for the development of new therapies for heart disease, with studies demonstrating the utility of adeno-associated virus (AAV) for delivering oligonucleotides in vivo. Two separate AAV2/9 virus' expressing antagonists of microRNAs (miRs) let-7a/let-c and miR-99/100 can induce proliferation of cardiomyocytes in the ischemic mouse heart for up to 3 months following a single injection. Transcriptomic and translational analysis on mice heart cells and tissues treated with viral delivered miR antagonists showed differences in the expression of genes and proteins involved in cardiac development, proliferation and muscle structure and function, implying that a similar regenerative effect, through targeting of these miRs, may occur in human cardiac myocytes and models of DMD.

Regeneration is a physiological reprogramming process. Regeneration can occur spontaneously in many vertebrates. Especially, lower vertebrates exhibit a remarkable regenerative ability (heart, liver, limbs, nervous system, etc.). Young mammals have remarkable regenerative capacity, which is lost over time (e.g. heart, kidney, etc.). Adult mammals (e.g., human) possess a limited regenerative capacity (e.g. liver), indicating the information for regenerating damaged tissues is still present in adult mammals and is just silenced. The adult heart muscle cannot regenerate. Permanent heart muscle loss leads to heart failure. A new treatment for heart disease can be develop by reactivating the regenerative programs in heart muscle. Inhibition of four specific, evolutionary and species conserved microRNAs (e.g., miR-99, miR-100, let-7a and/or let-7c) is a critical regulator of cardiomyocyte dedifferentiation and heart regeneration in Zebrafish which, unlike adult mammals, have retained the ability to regenerate heart tissue in response to injury.

The sequences of Let-7a/c and MicroRNA-99/100 from different species are compared in Table 1. Although human and murine adults fail to elicit an endogenous cardiomyocyte regenerative response after MI, in vivo manipulation of this molecular machinery, after injury in mammals can result in cardiomyocyte proliferation and improved heart function.

TABLE 1
CONSERVATION OF SEQUENCES OF LET-7A/C, MICRORNA-
99/100ACROSS SPECIES

| | | | SEQ |
|---|---|---|---|
| | Sequence | | ID |
| Species | name | Sequence | NO. |
| Dre | let-7a | UGAGGUAGUAGGUUGUAUAGUU | 2 |
| Mmu | let-7a-5p | UGAGGUAGUAGGUUGUAUAGUU | 2 |
| Rno | let-7a-5p | UGAGGUAGUAGGUUGUAUAGUU | 2 |
| Ssc | let-7a | UGAGGUAGUAGGUUGUAUAGUU | 2 |
| Ptr | let-7a | UGAGGUAGUAGGUUGUAUAGUU | 2 |
| Hsa | let-7a-5p | UGAGGUAGUAGGUUGUAUAGUU | 2 |
| Cfa | let-7a | UGAGGUAGUAGGUUGUAUAGUU | 2 |
| Dre | let-7c-5p | UGAGGUAGUAGGUUGUAUGGUU | 3 |
| Mmu | let-7c-5p | UGAGGUAGUAGGUUGUAUGGUU | 3 |
| Rno | let-7c-5p | UGAGGUAGUAGGUUGUAUGGUU | 3 |
| Ssc | let-7c | UGAGGUAGUAGGUUGUAUGGUU | 3 |
| Ptr | let-7c | UGAGGUAGUAGGUUGUAUGGUU | 3 |
| Hsa | let-7c-5p | UGAGGUAGUAGGUUGUAUGGUU | 3 |
| Cfa | let-7c | UGAGGUAGUAGGUUGUAUGGUU | 3 |
| Dre | miR-99 | AACCCGUAGAUCCGAUCUUGUG | 4 |
| Mmu | miR-99a-5p | AACCCGUAGAUCCGAUCUUGUG | 4 |
| Rno | miR-99a-5p | AACCCGUAGAUCCGAUCUUGUG | 4 |
| Cfa | miR-99a | AACCCGUAGAUCCGAUCUUGU | 5 |
| Ssc | miR-99a | AACCCGUAGAUCCGAUCUUGUG | 4 |
| Ptr | miR-99a | AACCCGUAGAUCCGAUCUUGUG | 4 |
| Hsa | miR-99a-5p | AACCCGUAGAUCCGAUCUUGUG | 4 |
| Dre | miR-100-5p | AACCCGUAGAUCCGAACUUGUG | 6 |
| Mmu | miR-100-5p | AACCCGUAGAUCCGAACUUGUG | 6 |
| Rno | miR-100-5p | AACCCGUAGAUCCGAACUUGUG | 6 |
| Ssc | miR-100 | AACCCGUAGAUCCGAACUUGUG | 6 |
| Ptr | miR-100 | AACCCGUAGAUCCGAACUUGUG | 6 |
| Hsa | miR-100-5p | AACCCGUAGAUCCGAACUUGUG | 6 |
| Cfa | miR-100 | | |

In Table 1, Dre: Danio rerio (zebrafish); Hsa: Homo sapiens (human); PtFGP-23,DNA,M Pan Troglodytes (Chimpanzee); Cfa: Canis Familiaris (dog); Ssc: Sus scrofa (minipig); Rno: Rattus norvegicus (rat); Mmu: Mus musculus (mouse). miR-100 is not found in dogs. let-7a and let-7c have one base difference at base 19. miR-99 and miR-100 have one base difference at base 16.

RNAi technology can take many forms, but it is typically implemented within a cell in the form of a base-pair short hairpin (sh) RNA (shRNA), which is processed into an approximately 20 base pair small interfering RNA through the endogenous miR pathway. Viral delivery of complementary sequences to miRs is a common approach. AAV vectors are optimal in cardiovascular muscle gene delivery since they a) contain no viral protein-coding sequences to stimulate an immune response, b) do not require active cell division for expression to occur and c) have a significant advantage over adenovirus vectors because of their stable, long-term expression of recombinant genes in myocytes in vivo. Viral delivery of genes is in development for the treatment of DMD and include AAV 1-gamma-sarcoglycan vector as a therapy for LGMD, recombinant (r) AAV2.5 vector for delivery of mini dystrophin, and rAAV, rhesus serotype 74.

The mechanism by which the miRNA antagonist functions to inhibit the activity of the target miRNA is not limited in any way. For example, a nucleic acid-based antagonist can form a duplex with the target miRNA sequences and prevent proper processing of the mature miRNA product from its precursor, or may prevent the mature miRNA from binding to its target gene, or may lead to degradation of pri-, pre-, or mature miRNA, or can act through some other mechanism.

By studying the mechanisms of let-7a/c and miR-100/99 inhibition in inducing heart regeneration in zebrafish and neonatal mice, it has been found that heart regeneration is a primarily cardiomyocyte-mediated process that occurs by dedifferentiation of mature cardiomyocytes followed by proliferation and further re-differentiation. Epigenetic remodeling and cell cycle control are two key steps controlling this regenerative process. Aguirre et al. (Cell Stem Cell. 2014; 15(5):589-604) reported a very relevant study, which investigated the underlying mechanism of heart regeneration and identified a series of miRs strongly involved in zebrafish heart regeneration. Not intending to being bound by any theories discussed herein, the study by Aguirre et al. (Cell Stem Cell. 2014; 15(5):589-604) is incorporated by reference in its entirety. Focus on those miRs that present significant expression changes and that were conserved across vertebrates, both in sequence and 3′ UTR binding sites, led to the identification of two miR families (miR-99/100, let-7a/c) clustered in two well-defined genomic locations. This finding was supported by a common role for the miR-99a/Let-7c-5p cluster in regulating vertebrate cardiomyogenesis. MIRANDA-based miR-UTR binding predictions showed a strong interaction for miR-99/100 with zebrafish FNTP (beta subunit of farnesyl-transferase) and SMARCA5 (SWI/SNF-related matrix associated actin-dependent regulator of chromatin subfamily a, member 5), linking the miR families to cell cycle and epigenetic control in cardiomyocytes. Increased expression of SMARCA5 and FNTP and GATA4 is associated with dedifferentiation of cardiomyocytes.

In addition to zebrafish, synergistic inhibition of miR-99/100 and Let-7a and let-7c is required for regeneration of human embryonic stem cell derived cardiomyocytes. In vivo intracardiac AAV2 delivery of miR inhibitors to mice with an MI restores cardiac function and reduces scarring. For example, miR-99/100 and let-7a/c levels are low during early mammalian heart development and promote quick cardiac mass growth, but increase exponentially during late development, with a corresponding decrease in FNTP and SMARCA5 protein levels to block further cardiomyocyte proliferation. Postmortem analysis of injured human heart tissue, suggests that these miRs constitute a conserved roadblock to cardiac regeneration in adults. RNA-seq transcriptomic analysis on neonatal mouse cardiomyocytes transduced two viral delivered antagonists to let-7a/c and miR-99/100 revealed differences in genes involved in epigenetic remodeling, demethylation, cardiac development, proliferation, and unexpectedly, metabolic pathways and muscle structural and function. Indeed, miR-let 7a/c and miR-99/100 inhibition targets 1072 and 47 genes, respectively. DNA synthesis was shown with an increase in BrDU incorporation. Nuclear ARK-2 and Anillin expression in mice also confirmed that cell division was the mechanism of cardiac muscle regeneration.

Compositions of the microRNA Antagonists and Vectors

Adult heart muscle cells cannot divide because they have high levels of miR-99/100 and let-7a/c inhibiting protein synthesis. By designing viral delivered JBT-miR2 and synthetic oligonucleotide drug JN-101 that independently lower the levels of miR-99/100 and let-7a/c, protein synthesis can be restored to the level required for cell division and re-differentiation similar to fetal heart muscle cells. Some compositions for reducing the levels of miR-99/100 and let-7a/c were described in PCT/US2020/056536, published as WO2021081006A1, which is herein incorporated by reference in its entirety.

In some embodiments, an adeno-associated virus (AAV) that delivers decoy inhibitors to all four of these microRNAs, was designed. In a preferred embodiment, JBT-miR2 is delivered by AAV 2/9 (JBT-miR2 (AAV 2/9)), which functions as microRNA inhibitors and treatment of IHD. JN-101 is a synthetic oligonucleotide also functions as microRNA inhibitors, treating DMD. Another AAV delivered vector is JN-210, which can also be delivered by AAV 2/9 (JN-210 (AAV 2/9)), treating familial hypertrophic cardiomyopathy (HCM). HCM affects about 500,000 patients.

microRNA Antagonists

In some embodiments, an miR antagonist comprises an antisense compound targeted to a miRNA. In some embodiments, an miR antagonist comprises a modified oligonucleotide having a nucleotide sequence that is complementary to the nucleotide sequence of a miRNA, or a precursor thereof. In other embodiments, an miR antagonist comprises a small molecule, or the like that interferes with or inhibits the activity of an miRNA. In some embodiments, an miR antagonist is a miR-99a antagonist. In some embodiments, an miR antagonist is a miR-100-5p antagonist. In some embodiments, an miR antagonist is a miR-Let-7a-5p antagonist. In some embodiments, an miR antagonist is a miR-Let-7c-5p antagonist.

The plurality of miR antagonists can include 4, 5, 6, 7, 8, 9, 10, 15, 20, 30, 40, 50 miR antagonists or a number of antagonists that is within a range defined by any two of the aforementioned values. In some embodiments, the plurality of miR antagonists includes one or more selected from miR-99a antagonists, miR-100-5p antagonists, miR-Let-7a-5p antagonists, miR-Let-7c-5p antagonists, and combinations thereof. In some embodiments, the numbers of each miR antagonist group are the same in the plurality of miR antagonists. In some embodiments, the numbers of each miR antagonist group are not the same in the plurality of miR antagonists.

The therapeutic composition can comprise a nucleotide sequence having at least 80% identity, at least 85%, at least 90% identity, or at least 95% identity to SEQ ID NO: 1. The therapeutic composition can comprise a nucleotide sequence of SEQ ID NO: 1, or a nucleotide sequence having no more than 10 (e.g., 0, 1, 2, 3, 4, 5, 6, 7, 8, 9, or 10) mismatches to the nucleotide sequence of SEQ ID NO: 1. The therapeutic composition can comprise at least two nucleotide sequences selected from: (i) a nucleotide sequence having at least 80% identity, at least 90% identity, or at least 95% identity to SEQ ID NOs: 14-18, (ii) a nucleotide sequence having at least 80% identity, at least 90% identity, or at least 95% identity to SEQ ID NOs: 19-23, (iii) a nucleotide sequence having at least 80% identity, at least 90% identity, or at least 95% identity to SEQ ID NOs: 24-31, and (iv) a nucleotide sequence having at least 80% identity, at least 90% identity, or at least 95% identity to SEQ ID NOs: 26-33. JRX0104, let-7 a/c antagomiR 5′-ATACAACCTACTACCTC-3′ (SEQ ID NO: 17) and JRX0116 miR-99/100 antagomiR 5′-GATCGGATCTACGGGT-3′ (SEQ ID NO: 26), that can be administered together, are collectively known as JN-101. The efficacy and safety were tested in mice and described in WO2021081006A1, which is herein incorporated by reference by its entirety. The therapeutic composition comprises a nucleotide sequence of SEQ ID NOs: 17 and 26, or a nucleotide sequence having no more than 10 (e.g., 0, 1, 2, 3, 4, 5, 6, 7, 8, 9, or 10) mismatches to the nucleotide sequence of SEQ ID NOs: 17 and 26.

Chemical Modifications to the microRNA Antagonists

miR antagonists described herein can have one or more chemical modifications. Suitable chemical modifications include, but are not limited to, modifications to a nucleobase, a sugar, and/or an internucleoside linkage. A modified nucleobase, sugar, and/or internucleoside linkage may be selected over an unmodified form because of desirable properties such as, for example, enhanced cellular uptake, enhanced affinity for other oligonucleotides or nucleic acid targets and increased stability in the presence of nucleases. Accordingly, in some embodiments of the compositions disclosed herein, at least one of the anti-miRs includes one or more chemical modifications selected from the group consisting of a modified internucleoside linkage, a modified nucleotide, and a modified sugar moiety, and combinations thereof.

The one or more chemical modifications include a modified internucleoside linkage. Generally, a modified internucleoside linkage can be any internucleoside linkage known in the art. Non-limiting examples of suitable modified internucleoside linkage include a phosphorothioate, 2′-Omethoxyethyl (MOE), 2′-fluoro, alkylphosphonate, phosphorodithioate, alkylphosphonothioate, phosphoramidate, carbamate, carbonate, phosphate triester, acetamidate, carboxymethyl ester, and combinations thereof. In some embodiments, the modified internucleoside linkage comprises a phosphorus atom. In some embodiments, the modified internucleoside linkage does not comprise a phosphorus atom. In certain such embodiments, an internucleoside linkage is formed by a short chain alkyl internucleoside linkage. In certain such embodiments, an internucleoside linkage is formed by a cycloalkyl internucleoside linkages. In certain such embodiments, an internucleoside linkage is formed by a mixed heteroatom and alkyl internucleoside linkage. In certain such embodiments, an internucleoside linkage is formed by a mixed heteroatom and cycloalkyl internucleoside linkages. In certain such embodiments, an internucleoside linkage is formed by one or more short chain heteroatomic internucleoside linkages. In certain such embodiments, an internucleoside linkage is formed by one or more heterocyclic internucleoside linkages. In certain such embodiments, an internucleoside linkage has an amide backbone. In certain such embodiments, an internucleoside linkage has mixed N, O, S and CH₂ component parts. In some embodiments, at least one of the anti-miRs includes a modified internucleoside linkage which is a phosphorothioate internucleoside linkage.

In some embodiments, at least one of the one or more chemical modifications includes a modified nucleotide. A modified nucleotide can generally be any modified nucleotide and can be for example, a locked nucleic acid (LNA) chemistry modification, a peptide nucleic acid (PNA), an arabino-nucleic acid (FANA), an analogue, a derivative, or a combination thereof. In some embodiments, the modified nucleotide comprises 5-methylcytosines. In some embodiments, a modified nucleotide is selected from 5-hydroxymethyl cytosine, 7-deazaguanine and 7- deazaadenine. In certain embodiments, the modified nucleotide is selected from 7-deaza-adenine, 7-deazaguanosine, 2-aminopyridine and 2-pyridone. In certain embodiments, the modified nucleotide is selected from 5-substituted pyrimidines, 6-azapyrimidines and N-2, N-6 and 0-6 substituted purines, including 2-aminopropyladenine, 5-propynyluracil and 5-propynylcytosine. In certain embodiments, a modified nucleotide comprises a polycyclic heterocycle. In certain embodiments, a modified nucleotide comprises a tricyclic heterocycle. In certain embodiments, a modified nucleotide comprises a phenoxazine derivative. In certain embodiments, the phenoxazine can be further modified to form a nucleobase known in the art as a G-clamp.

In some embodiments, the modified nucleotide includes a locked nucleic acid (LNA). In some embodiments, the one or more chemical modifications include at least one locked nucleic acid (LNA) chemistry modifications to enhance the potency, specificity and duration of action and broaden the routes of administration of oligonucleotides. This can be achieved by substituting some of the nucleobases in a base nucleotide sequence by LNA nucleobases. The LNA modified nucleotide sequences can have a size similar to the parent nucleobase or can be larger or preferably smaller. In some embodiments, the LNA-modified nucleotide sequences contain less than about 70%, less than about 65%, more preferably less than about 60%, less than about 55%, most preferably less than about 50%, less than about 45% LNA nucleobases and that their sizes are between about 5 and 25 nucleotides, more preferably between about 12 and 20 nucleotides. In some embodiments, the LNA is incorporated at one or both ends of the modified anti-miR.

In some embodiments, the one or more chemical modifications include at least one modified sugar moiety. In some embodiments, a sugar modified nucleoside is a 2′-modified nucleoside, wherein the sugar ring is modified at the 2′ carbon from natural ribose or 2′-deoxy-ribose. In some embodiments, a 2′-modified nucleoside has a bicyclic sugar moiety. In certain such embodiments, the bicyclic sugar moiety is a D sugar in the alpha configuration. In certain such embodiments, the bicyclic sugar moiety is a D sugar in the beta configuration. In certain such embodiments, the bicyclic sugar moiety is an L sugar in the alpha configuration. In certain such embodiments, the bicyclic sugar moiety is an L sugar in the beta configuration.

The bicyclic sugar moiety can comprise a bridge group between the 2′ and the 4′-carbon atoms. For example, the bridge group can be from 1 to 8 linked biradical groups, 1 to 4 linked biradical groups, or 2 or 3 linked biradical groups. In some embodiments, the bicyclic sugar moiety comprises 2 linked biradical groups. In some embodiments, a linked biradical group is selected from —O—, —S—, —N(R₁)—, —C(R₁)(R₂)—, —C(R₁)═C(R₁)—, —C(R₁)═N—, —C(═NR₁)—, —Si(R₁)(R₂)—, —S(═O)₂—, —S(═O)—, —C(═O)— and —C(═S)—; where each R₁ and R₂ is, independently, H, hydroxyl, C₁-C₁₂ alkyl, substituted C₁-C₁₂ alkyl, C₁-C₁₂ alkenyl, substituted C₁-C₁₂ alkenyl, C₁-C₁₂ alkynyl, substituted C₁-C₁₂ alkynyl, C₅-C₂o aryl, substituted C₅-C₂o aryl, a heterocycle radical, a substituted heterocycle radical, heteroaryl, substituted heteroaryl, C₅-C₇ alicyclic radical, substituted C₅-C₇ alicyclic radical, halogen, substituted oxy (—O—), amino, substituted amino, azido, carboxyl, substituted carboxyl, acyl, substituted acyl, CN, thiol, substituted thiol, sulfonyl (S(═O)₂—H), substituted sulfonyl, sulfoxyl (S(═O)—H) or substituted sulfoxyl; and each substituent group is, independently, halogen, C₁-C₁₂ alkyl, substituted C₁-C₁₂ alkyl, C₁-C₁₂ alkenyl, substituted C₁-C₁₂ alkenyl, C₁-C₁₂ alkynyl, substituted C₁-C₁₂ alkynyl, amino, substituted amino, acyl, substituted acyl, C₁-C₁₂ aminoalkyl, C₁-C₁₂ aminoalkoxy, substituted C₁-C₁₂ aminoalkyl, substituted C₁-C₁₂ aminoalkoxy or a protecting group.

In some embodiments, the bicyclic sugar moiety is bridged between the 2′ and 4′ carbon atoms with a biradical group selected from —O—(CH₂)_{P}—, —O—CH₂— —O—CH₂CH₂—, —O—CH(alkyl)-, —NH—(CH₂)_{P}—, —N(alkyl)-(CH₂)_{P}—, —O—CH(alkyl)-, —(CH(alkyl))-(CH₂)_{P}—, —NH—O—(CH₂)_{P}—, —N(alkyl)-O—(CH₂)_{P}—, or —O—N(alkyl)-(CH₂)_{P}—, wherein p is 1, 2, 3, 4 or 5 and each alkyl group can be further substituted. In some embodiments, p is 1, 2 or 3.

In some embodiments, a 2′-modified nucleoside comprises a 2′-substituent group selected from halo, allyl, amino, azido, SH, CN, OCN, CF₃, OCF₃, O—, S—, or N(Rₘ)-alkyl; O—, S—, or N(Rₘ)-alkenyl; O—, S— or N(Rₘ)-alkynyl; O-alkylenyl-O-alkyl, alkynyl, alkaryl, aralkyl, O-alkaryl, O-aralkyl, O(CH₂)₂SCH₃, O—(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some embodiments, a 2′-modified nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), —O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In some embodiments, a 2′-modified nucleoside comprises a 2′-substituent group selected from F, OCF₃, O—CH₃, OCH₂CH₂OCH₃, 2′-O(CH₂)₂SCH₃, O—(CH₂)₂—O—N(CH₃)₂, O(CH₂)₂O(CH₂)₂N—(CH₃)₂, and O—CH₂—C(═O)—N(H)CH₃.

In some embodiments, a 2′-modified nucleoside comprises a 2′-substituent group selected from F, O—CH₃, and OCH₂CH₂OCH₃.

In some embodiments, a sugar-modified nucleoside is a 4′-thio modified nucleoside. In certain embodiments, a sugar-modified nucleoside is a 4′-thio-2′-modified nucleoside. A 4′-thio modified nucleoside has a P-D-ribonucleoside where the 4′-0 replaced with 4′-S. A 4′-thio-2′-modified nucleoside is a 4′-thio modified nucleoside having the 2′-OH replaced with a 2′-substituent group. Suitable 2′-substituent groups include 2′-OCH₃, 2′-O—(CH₂)₂—OCH₃, and 2′-F.

In some embodiments, the modified sugar moiety is a 2′-O-methoxyethyl modified sugar moiety, a 2′-methoxy modified sugar moiety, a 2′-O-alkyl modified sugar moiety, a bicyclic sugar moiety, or a combination thereof. In some embodiments, the modified sugar moiety comprises a 2′-O-methyl sugar moiety.

### Cloning and Expression Vector

In some embodiments, one or more of the miR antagonists described herein can be encoded by and/or expressed from a cloning vector or an expression vector. In some embodiments, a vector is composed of DNA. In some embodiments, a vector is composed of RNA. Vectors are preferably capable of autonomous replication, by having the gene encoding the miR antagonists operably linked to a promoter.

The therapeutic composition can comprise a viral and/or non-viral vector comprising the nucleotide sequence. In some embodiments, the cloning vector or expression vector is a viral vector. A viral vector is capable of directing expression of a gene, a protein or proteins encoded by one or more genes carried by the vector when it is present in the appropriate environment. Examples for viral vectors include, but are not limited to adenovirus vectors, adeno-associated virus (AAV) vectors, retrovirus vectors, lentiviral vectors, herpes virus vectors, phages, and poxvirus vectors.

In some embodiments, the viral vector is a lentiviral vector or an adeno-associated viral (AAV) vector or any serotype. As used herein, the term “serotype” or “serovar” is a distinct variation within a species of bacteria or virus or among immune cells of different individuals. These microorganisms, viruses, or cells are classified together based on their cell surface antigens, allowing the epidemiologic classification of organisms to the sub-species level. Generally, the AAV vector can be any existing AAV vectors and can be, for example, an AAV vector selected from serotype 1, 2, 3, 4, 5, 6, 7, 8, 9, and 10 or chimeric AAV derived thereof, which will be even better suitable for high efficiency transduction in the tissue of interest. Upon transfection, AAV elicits only a minor immune reaction (if any) in the host. Therefore, AAV vector is highly suited for gene therapy approaches. It has been reported that, for transduction in mice, AAV serotype 6 and AAV serotype 9 are particularly suitable. For gene transfer into a human, AAV serotypes 1, 6, 8 and 9 are generally preferred. It has been also assumed that the capacity of AAV for packaging a therapeutic gene is limited to approximately 4.9 kb, while longer sequences lead to truncation of AAV particles. In some embodiments, the AAV vector is an AAV2/9 vector, e.g., AAV2 inverted terminal repeat (ITR) sequences cross-packaged into AAV capsid.

The pAV-U6-GFP vector can be used as the basic cloning vector. Vector genomes with AAV2 ITR sequences can be cross packaged into AAV2/9 capsids via triple transfection of AAV-293 cells, then purified by iodixanol gradient centrifugation. The structure of pAV-U6-GFP vector is described in US20220307028A1, which is hereby incorporated by reference in its entirety.

In some embodiments, the cloning vector or expression vector disclosed herein includes a nucleotide sequence having, or having about, 80%, 85%, 90%, 91%, 92%, 93%, 94%, 95%, 96%, 97%, 98%, 99%, 100%, or a range between any two of these values, sequence identity to the full sequence of JBT-miR2-ADD (SEQ ID NO: 1).

In some embodiment, the promoter operably linked to the gene encoding the miR antagonists is an RNA polymerase III promoter. Examples of RNA polymerase III promoter include but not limited to U6 promoter, H1 promoter and 7SK promoter. The U6 promoter can be selected from a U6-9 promoter, a U6-1 promoter and U6-8 promoter.

The vector can have at least two promoters. In some embodiments, the at least two promoters are the same promoter. In some embodiments, the at least two promoters are different promoters. In a preferred embodiment, the vector can have a U6 promoter, and an H1 promoter. In some embodiments, the U6 promoter is operably linked to the gene encoding one miR antagonist and the H1 promoter is operably linked to another miR antagonist.

### Therapeutic Compositions and Pharmaceutical Formulations

While it is possible for the agents to be administered as the raw substances, it is preferable, in view of their potency, to present them as a pharmaceutical formulation. Thus, in some embodiments of the compositions disclosed herein, the composition is further formulated into a pharmaceutical formulation. For example, a pharmaceutical formulation can comprise an anti-miR antagonist disclosed herein and a sterile aqueous solution. For example, the pharmaceutical formulations of the present disclosure for human use comprise the agent, together with one or more acceptable carriers therefor and optionally other therapeutic ingredients. The carrier(s) must be “acceptable” in the sense of being compatible with the other ingredients of the formulation and not deleterious to the recipient thereof or deleterious to the inhibitory function of the active agent. Desirably, the pharmaceutical formulations should not include oxidizing agents and other substances with which the agents are known to be incompatible.

Accordingly, some embodiments disclosed herein relate to pharmaceutical formulations that include a therapeutic composition described herein and a pharmaceutically acceptable carrier. The formulations can also comprise additional ingredients such as diluents, stabilizers, excipients, and adjuvants.

Buffers can also be included in the pharmaceutical formulations to provide a suitable pH value for the formulation. Suitable such materials include sodium phosphate and acetate. Sodium chloride or glycerin may be used to render a formulation isotonic with the blood. If desired, the formulation may be filled into the containers under an inert atmosphere such as nitrogen or may contain an anti-oxidant and are conveniently presented in unit dose or multi-dose form, for example, in a sealed ampoule.

The carriers, diluents and adjuvants can include antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

Generally, the pharmaceutical formulations disclosed herein can be prepared by any one of the methods and techniques known in the art. For example, solid dosage forms can be prepared by wet granulation, dry granulation, direct compression, and the like. In some embodiments, the solid dosage forms of the present disclosure may be coated or otherwise compounded to provide a dosage form affording the advantage of prolonged action. For example, the tablet or pill can comprise an inner dosage and an outer dosage component, the latter being in the form of an envelope over the former. In some embodiments, the two components can be separated by an enteric layer, which serves to resist disintegration in the stomach and permit the inner component to pass intact into the duodenum or to be delayed in release. In these instances, a variety of materials can be used for such enteric layers or coatings, such materials including a number of polymeric acids and mixtures of polymeric acids with such materials as shellac, cetyl alcohol, and cellulose acetate.

Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

For example, dosage regimens can be adjusted to provide the optimum desired response. For example, a single dose can be administered, or several divided doses can be administered over time, or the dose may be proportionally reduced or increased as indicated by the exigencies of the therapeutic situation. It is especially advantageous to formulate parenteral compositions and formulations in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure are dictated by and directly dependent on (a) the unique characteristics of the therapeutic agent and the particular therapeutic or prophylactic effect to be achieved, and (b) the limitations inherent in the art of compounding such an active compound for the treatment of sensitivity in individuals.

The expression vectors and/or the miRNA antagonists disclosed herein can be administered to a subject (e.g., a human) in need thereof. For example, a therapeutically effective amount of the recombinant viruses can be administered to the subject by via routes standard in the art. Non-limiting examples of the route include intramuscular, intravaginal, intravenous, intraperitoneal, subcutaneous, epicutaneous, intradermal, rectal, intraocular, pulmonary, intracranial, intraosseous, oral, buccal, or nasal. In some embodiments, the recombinant virus is administered to the subject by intramuscular injection. In some embodiments, the recombinant virus is administered to the subject by intravaginal injection. In some embodiments, the expression vectors and/or the miRNA antagonists is administered to the subject by the parenteral route (e.g., by intravenous, intramuscular or subcutaneous injection), by surface scarification or by inoculation into a body cavity of the subject. In some embodiments, the expression vectors and/or the miRNA antagonists are administered to muscle cells such as, cardiac muscle cells.

When administering these small miR oligonucleotide antagonists by injection, the administration can be by continuous infusion, or by single or multiple boluses. Typically, it is desirable to provide the recipient with a dosage of the molecule which is in the range of from about 1 μg/kg to 10 mg/kg (amount of agent/body weight of patient), although a lower or higher dosage can also be administered.

In some embodiments, it may be desirable to target delivery of a therapeutic to the heart, while limiting delivery of the therapeutic to other organs. This may be accomplished by any one of a number of methods known in the art. In some embodiments, delivery to the heart of a therapeutic composition or pharmaceutical formulation described herein comprises coronary artery infusion. In certain embodiments, coronary artery infusion involves inserting a catheter through the femoral artery and passing the catheter through the aorta to the beginning of the coronary artery. In yet some other embodiments, targeted delivery of a therapeutic to the heart involves using antibody-protamine fusion proteins, such as those previously describe (Song E et al., Nature Biotechnology, 2005), to deliver the small miR oligonucleotide antagonists disclosed herein.

Actual administration of the expression vectors and/or the miRNA antagonists can be accomplished by using any physical method that will transport the expression vectors and/or the miRNA antagonists into the target tissue of the subject. For example, the expression vectors and/or the miRNA antagonists can be injected into muscle, the bloodstream, and/or directly into the liver. Pharmaceutical formulations can be prepared as injectable formulations or as topical formulations to be delivered to the muscles by transdermal transport.

For intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of the expression vectors and/or the miRNA antagonists as a free acid (DNA contains acidic phosphate groups) or a pharmacologically acceptable salt can be prepared in water suitably mixed with a surfactant such as hydroxpropylcellulose. A dispersion of the expression vectors and/or the miRNA antagonists can also be prepared in glycerol, liquid polyethylene glycols and mixtures thereof and in oils. Under ordinary conditions of storage and use, these preparations contain a preservative to prevent the growth of microorganisms.

The expression vectors and/or the miRNA antagonists to be used can be utilized in liquid or freeze-dried form (in combination with one or more suitable preservatives and/or protective agents to protect the virus during the freeze-drying process). For gene therapy (e.g of neurological disorders which may be ameliorated by a specific gene product) a therapeutically effective dose of the recombinant virus expressing the therapeutic protein is administered to a host in need of such treatment. The use of the expression vectors and/or the miRNA antagonists disclosed herein in the manufacture of a medicament for inducing immunity in, or providing gene therapy to, a host is within the scope of the present application.

In instances where human dosages for the expression vectors and/or the miRNA antagonists have been established for at least some conditions, those same dosages, or dosages that are between about 0.1% and 500%, more preferably between about 25% and 250% of the established human dosage can be used. Where no human dosage is established, as will be the case for newly-discovered pharmaceutical formulations, a suitable human dosage can be inferred from ED₅₀ or ID₅₀ values, or other appropriate values derived from in vitro or in vivo studies, as qualified by toxicity studies and efficacy studies in animals. Typically, normalization to body surface area is an appropriate method for extrapolating doses between species. The method of conversion between species is described in a publication entitled “Estimating the Maximum Safe Starting Dose in Initial Clinical Trials for Therapeutics in Adult Healthy Volunteers” by U.S. Department of Health and Human Services, which is herein incorporated by reference by its entirety.

A therapeutically effective amount of the expression vectors and/or the miRNA antagonists can be administered to a subject at various points of time. For example, the expression vectors and/or the miRNA antagonists can also be administered to the subject prior to, during, or after the occurrence of a heart disease.

In some embodiments, a pharmaceutical kit is provided, wherein the kit comprises: any of the forgoing the therapeutic compositions and pharmaceutical formulations, and written information (a) indicating that the formulation is useful for inhibiting, in myocardial cells, such as, for example cardiomyocytes, the function of a gene associated with the heart disease and/or (b) providing guidance on administration of the pharmaceutical formulation.

### Methods of Treating Heart Disease and Regenerating Heart Muscle

Oxidative stresses associated with reperfusion can cause damage to the affected tissues or organs. Ischemia-reperfusion injury is characterized biochemically by a depletion of oxygen during an ischemic event followed by reoxygenation and the concomitant generation of reactive oxygen species during reperfusion. In some embodiments, the compositions provided herein are administered at the time of reperfusion. At the time of reperfusion can range from two hours before to 2 hours after reperfusion, as well as right at the same time of reperfusion. The compositions provided herein can be administered at the same time as, for example, a thrombolytic agent is administered, or at the time of performing a surgical intervention to eliminate the clot obstructing the blood flow.

In some embodiments, administering the therapeutic composition occurs before the onset of the cardiac ischemic event. In some embodiments, administering the therapeutic composition occurs during the cardiac ischemic event. The therapeutic composition can be administered about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, about 72 hours, or about 96 hours prior to reperfusion of ischemic cardiac tissue. In some embodiments, administering the therapeutic composition occurs concurrent with reperfusion of ischemic cardiac tissue. In some embodiments, administering the therapeutic composition occurs after reperfusion of ischemic cardiac tissue. The therapeutic composition can be administered about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, about 72 hours, about 96 hours, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, or about 20 days, after reperfusion of ischemic cardiac tissue.

The therapeutic composition can comprise a plurality of microRNA (miR) antagonists, the administration can comprise subcutaneous administration, systemic administration, and/or intra-coronary administration, and the therapeutic composition can be administered at a dose of about 0.0001 mg/kg to 100 mg/kg (e.g., about 0.08 mg/kg, about 0.24 mg/kg, about 0.81 mg/kg, about 1.22 mg/kg, about 2.44 mg/kg, about 3.25 mg/kg, about 4.06 mg/kg, about 4.89 mg/kg, about 5.69 mg/kg, about 6.50 mg/kg, about 7.32 mg/kg, or about 8.13 mg/kg). The therapeutic composition can comprise a plurality of microRNA (miR) antagonists, the administration can comprise intra-ventricular administration and/or intra-myocardial administration, and the therapeutic composition can be administered at a dose of about 0.0001 mg/kg to 100 mg/kg (e.g., about 0.004 mg/kg, about 0.012 mg/kg, about 0.0405 mg/kg, about 0.061 mg/kg, about 0.122 mg/kg, about 0.1625 mg/kg, about 0.203 mg/kg, about 0.2445 mg/kg, about 0.2845 mg/kg, about 0.325 mg/kg, about 0.366 mg/kg, or about 0.4065 mg/kg). In some embodiments, subcutaneous administration of the therapeutic composition yields increased survival and reduced incidence of cardiac thrombus as compared to intravenous administration of the therapeutic composition.

The therapeutic composition can comprise a viral vector, and the administration can comprise intravenous systemic administration and/or intra-coronary administration at a dose of about 1.0×10⁵ vg/kg to 1.0×10¹⁹ vg/kg (e.g., about 2.5×10¹² vg (viral genome)/kg, about 2.5×10¹³ vg/kg, about 2.5×10¹⁴ vg/kg, or about 2.5×10¹⁵ vg/kg). The therapeutic composition can comprise a viral vector, the administration can comprise intra-ventricular administration and/or intra-myocardial administration, and the therapeutic composition can be administered at a dose of about 1.0×10⁵ vg/kg to 1.0×10¹⁹ vg/kg (e.g., about 0.125×10¹² vg/kg, about 0.125×10¹³ vg/kg, about 0.125×10¹⁴ vg/kg, or about 0.125×10¹⁵ vg/kg).

The therapeutic composition can be a pharmaceutical composition. The subject can be a mammal (e.g., a human). The method of preventing, inhibiting, reducing, or treating ischemic heart injury provided herein comprises administering a therapeutic composition to a subject before, during, and/or after a cardiac ischemic event, wherein the therapeutic composition is administrated in a first dose and a second dose separated by a dosing interval.

The first dose can be administered at a dose of about 1.0×10⁵ vg/kg to 1.0×10¹⁹ vg/kg (e.g., about 1.0×10¹⁰ vg/kg, 2.5×10¹⁰ vg/kg, 2.5×10¹¹ vg/kg, 2.5×10¹² vg/kg, about 2.5×10¹³ vg/kg, about 2.5×10¹⁴ vg/kg, or about 2.5×10¹⁵ vg/kg). The second dose can be at least 50% (e.g., 50%, 60%, 70%, 80%, 90%, or 100%) of the first dose.

The second dose can be administered at a dose of about 1.0×10⁵ vg/kg to 1.0×10¹⁹ vg/kg (e.g., about 1.0×10¹⁰ vg/kg, 2.5×10¹⁰ vg/kg, 2.5×10¹¹ vg/kg, 1.7×10¹² vg, 2.5×10¹² vg/kg, about 2.5×10¹³ vg/kg, about 2.5×10¹⁴ vg/kg, or about 2.5×10¹⁵ vg/kg). The second dose can be administrated at the same total amount per subject as the first dose.

The second dose can be administered to the subject in the same or different manner as the first dose. The first dose and/or the second dose can be administrated through routes including intracardiac, intramuscular, intravaginal, intravenous, intra-myocardial, intra-ventricular, subcutaneous, systemic, intra-coronary, intraperitoneal, subcutaneous, epicutaneous, intradermal, rectal, intraocular, pulmonary, intracranial, intraosseous, oral, buccal, or nasal. In some embodiments, the first dose is administrated through intracardiac (IC) infusion. The first dose can be administrated at a rate of 0.1/mL/min-10/m/min (e.g., 0.1/m/min, 1/mL/min, 2/mL/min, 3/m/min, 4/m/min, 5/mL/min, 6/mL/min, 7/mL/min, 8/m/min, 9/mL/min, or 10/m/min), if administrated through intracardiac (IC) infusion. Preferably, the second dose can be administrated intravenously (IV) (e.g., IV bolus injection). The administration of the second dose through IV injection can broaden clinical application to patients, because in the chronic heart failure (CHF) setting where percutaneous coronary intervention (PCI) is not possible.

The second dose can be administrated to the subject about 1 day to about 1000 days (e.g., about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, and/or about 20 days, about 30, about 40 days, about 50 days, about 60 days, about 70 days, about 80 days, and/or about 90 days) after the administration of the first dose. The second dose can be administrated before and/or after the first dose is cleared from the subject. The second dose can be administrated when at least 50% (e.g., 60%, 70%, 80%, 90%, and/or 100%) of the first dose is cleared from the subject. The second dose can be administrated at a timing that it does not instigate immune response.

The method can comprise: additional repeated administration of the therapeutic composition to the subject. The repeated administration can comprise administration of one or more additional doses of the therapeutic composition to the subject. The number of additional doses can vary, and can range from 1 additional dose to 100 additional doses. The one or more additional doses can the same, larger, or smaller, than the initial administration. Reperfusion of ischemic cardiac tissue can comprise a percutaneous coronary intervention, coronary artery bypass grafting, thrombolytic therapy, anti-platelet therapy, heparin, warfarin, fibrinolytics, oxygen therapy, a vasodilator, pain medication, a beta blocker, an angiotensin-converting enzyme (ACE) inhibitor, an angiotensin receptor blocker (ARB), a glycoprotein antagonist, a statin, an aldosterone antagonist, an implantable cardiac defibrillator (ICD), or any combination thereof.

In some embodiments, the subject has or is suspected of having a cardiac disease. The cardiac disease can be myocardial infarction, ischemic heart disease, dilated cardiomyopathy, heart failure (e.g., congestive heart failure), ischemic cardiomyopathy, hypertrophic cardiomyopathy, restrictive cardiomyopathy, alcoholic cardiomyopathy, viral cardiomyopathy, tachycardia-mediated cardiomyopathy, stress-induced cardiomyopathy, amyloid cardiomyopathy, arrhythmogenic right ventricular dysplasia, left ventricular noncompaction, endocardial fibroelastosis, aortic stenosis, aortic regurgitation, mitral stenosis, mitral regurgitation, mitral prolapse, pulmonary stenosis, pulmonary regurgitation, tricuspid stenosis, tricuspid regurgitation, congenital disorder, genetic disorder, or any combination thereof. The subject can be affected by a condition selected from alcoholic cardiomyopathy, coronary artery disease, congenital heart disease, nutritional diseases affecting the heart, ischemic cardiomyopathy, hypertensive cardiomyopathy, valvular cardiomyopathy, inflammatory cardiomyopathy, cardiomyopathy secondary to a systemic metabolic disease, dilated cardiomyopathy (DCM), hypertrophic cardiomyopathy (HCM), arrhythmogenic right ventricular cardiomyopathy (ARVC), restrictive cardiomyopathy (RCM), noncompaction cardiomyopathy, supravalvular aortic stenosis (SVAS), vascular scarring, atherosclerosis, chronic progressive glomerular disease, glomerulosclerosis, progressive renal failure, vascular occlusion, hypertension, stenosis, diabetic retinopathy, or any combination thereof.

The cardiac ischemic reperfusion injury can comprise cardiac ischemic damage, cardiac reperfusion injury, or a combination thereof. In some embodiments, the administration reduces cardiac ischemic damage, cardiac reperfusion injury, or a combination thereof, as compared to a control subject. In some embodiments, the administration reduces creatine kinase levels as compared to a control subject. The cardiac ischemic reperfusion injury can comprise injuries caused by the cardiac ischemia event, reperfusion injuries, or a combination thereof.

The cardiac ischemic event can comprise one or more of myocardial infarction, coronary artery bypass grafting (CABG), cardiac bypass surgery, cardiac transplantation, and angioplasty. The cardiac ischemic event can comprise a vascular interventional procedure employing a stent, laser catheter, atherectomy catheter, angioscopy device, beta or gamma radiation catheter, rotational atherectomy device, coated stent, radioactive balloon, heatable wire, heatable balloon, biodegradable stent strut, a biodegradable sleeve, or any combination thereof.

In some embodiments, the administration results in one or more of (1) increased survival as compared to a control subject, (2) improved kidney function of the subject as compared to a control subject, (3) a decrease in blood urea nitrogen (BUN) levels as compared to a control subject, (4) a reduced scarring in the left ventricle of the subject and/or improved regional wall motion in the left ventricle of the subject as compared to a control subject, (5) a decrease in end diastolic volume and/or end systolic volume as compared to a control subject, (6) an increase in ejection fraction as compared to a control subject, (7) an increase in the number of cardiomyocytes and/or mRNAs encoding proteins that are involved in differentiated cardiomyocyte muscle structure and function as compared to a control subject, (8) an increase in the mRNA levels and/or protein levels of one or more of Ank2, Kdm6a, Grk6, KM15, Adam22, Pfkp, Gorasp2, Ralgpsl, Inppll, Kdm3a, Kit, Sortl, Dvl2, Sema6d, Teadl, B4galnt2, Ltbp4, Osbpl9, Nfe2Il, Tnnt2, and Fhll as compared to a control subject, and (9) a decrease in the mRNA levels and/or protein levels of one or more of Asph, Map6, Zfpl20, Ctnndl, Eya3, Tnnt2, Kdm3a, Myol8a, Ncoa6, Slc25al3, Rpe, Ralgpsl, Gimapl, Myo5a, Zeb2, Arapl, Nt5c2, Phldbl, Ttn, Camta2, Mef2c, Slk, Uimcl, Mthfdll, Mtusl, Ythdcl, and Eif2ak4 as compared to a control subject, and (10) an increase in one of more of cardiomyocyte formation, cardiomyocyte proliferation, cardiomyocyte cell cycle activation, mitotic index of cardiomyocytes, myofilament density, borderzone wall thickness, or any combination thereof, as compared to a control subject, by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) at a time point about 5 minutes to about 365 days after administration (e.g., about 5 minutes, about 10 minutes, about 15 minutes, about 20 minutes, about 25 minutes, about 30 minutes, about 40 minutes, about 50 minutes, or about 60 minutes, about 1 day, about 2 days, about 4 days, about 6 days, about 8 days, about 10 days, about 20 days, about 30 days, about 40 days, about 50 days, about 60 days, about 80 days, about 100 days, about 120 days, about 140 days, about 160 days, about 180 days, about 200 days, about 220 days, about 240 days, about 260 days, about 280 days, about 300 days, about 320 days, about 340 days, about 360 days, about 365 days, or a number or a range between any of these values). In some embodiments, the administration induces endogenous cardiomyocyte regeneration. In some embodiments, the administration enhances cardiac function in the subject as compared to a control subject. Enhancing cardiac function can comprise one or more of (i) improving left ventricular function, (ii) improving fractional shortening, (iii) improving ejection fraction, (iv) reducing end-diastolic volume, (v) decreasing left ventricular mass, and (v) normalizing of heart geometry, or (vi) a combination thereof. In some embodiments, the administration has no significant effect on body weight and/or heart weight. In some embodiments, the administration does not cause one or more of arrhythmia, after contractions (AC), and contraction failure (CF).

The compositions provided herein can also be used to inhibit an ischemia or ischemia-reperfusion injury to a cell, tissue or organ, ex vivo, prior to a therapeutic intervention (e.g., a tissue employed in a graft procedure, an organ employed in an organ transplant surgery). For example, prior to transplant of an organ into a host individual (e.g., during storage or transport of the organ in a sterile environment), the organ can be contacted with compositions provided herein (e.g., bathed in a solution comprising the compositions provided herein) to inhibit ischemia or ischemia-reperfusion injury.

The methods provided herein can treat a disease or disorder associated with one or more FHF1 mutations and/or one or more TNNT2 mutations. In some embodiments, the therapeutic composition increases the mRNA levels and/or protein levels of FHF1 and/or TNNT2. The disease or disorder can be a muscular dystrophy disorder or a muscular dystrophy-like muscle disorder. The muscular dystrophy disorder can be associated with Amyotrophic Fateral Sclerosis (AFS), Charcot-Marie-Tooth Disease (CMT), Congenital Muscular Dystrophy (CMD), Duchenne Muscular Dystrophy (DMD), Emery-Dreifuss Muscular Dystrophy (EDMD), Inherited and Endocrine Myopathies, Metabolic Diseases of Muscle, Mitochondrial Myopathies (MM), Myotonic Muscular Dystrophy (MMD), Spinal-Bulbar Muscular Atrophy (SBMA), or a combination thereof. The disease or disorder can be Limb girdle muscular dystrophy, X-linked myopathy with postural muscle atrophy (XMPMA), Reducing body myopathy (RBM), Scapuloperoneal (SP) syndrome, or any combination thereof. The disease or disorder can be hypertrophic cardiomyopathy (HCM), restrictive cardiomyopathy (RCM), dilated cardiomyopathy (DCM), or any combination thereof. The hypertrophic cardiomyopathy can be familial hypertrophic cardiomyopathy.

In some embodiments, the compositions disclosed herein exhibit a renal therapeutic effect. In some embodiments, the renal therapeutic effect comprises a renal protective effect or renal prophylactic effect. The methods provided herein can treat a kidney condition associated with a function of the subject's kidneys. The kidney condition can be selected from acute kidney diseases and disorders (AKD), acute kidney injury, acute and rapidly progressive glomerulonephritis, acute presentations of nephrotic syndrome, acute pyelonephritis, acute renal failure, idiopathic chronic glomerulonephritis, secondary chronic glomerulonephritis, chronic heart failure, chronic interstitial nephritis, chronic kidney disease (CKD), chronic liver disease, chronic pyelonephritis, diabetes, diabetic kidney disease, fibrosis, focal segmental glomerulosclerosis, Goodpasture's disease, diabetic nephropathy, hereditary nephropathy, interstitial nephropathy, hypertensive nephrosclerosis, IgG4-related renal disease, interstitial inflammation, lupus nephritis, nephritic syndrome, partial obstruction of the urinary tract, polycystic kidney disease, progressive renal disease, renal cell carcinoma, renal fibrosis, graft versus host disease after renal transplant, and vasculitis. The methods provided herein can protect a kidney of a subject from an injury associated with one or more of surgery, radiocontrast imaging, radiocontrast nephropathy, cardiovascular surgery, cardiopulmonary bypass, extracorporeal membrane oxygenation (ECMO), balloon angioplasty, induced cardiac or cerebral ischemic-reperfusion injury, organ transplantation, kidney transplantation, sepsis, shock, low blood pressure, high blood pressure, kidney hypoperfusion, chemotherapy, drug administration, nephrotoxic drug administration, blunt force trauma, puncture, poison, or smoking. The therapeutic composition can be administered in combination with a renal therapeutic agent, such as, for example, those selected from the group comprising dexamethasone, a steroid, budesonide, triamcinolone acetonide, an anti-inflammatory agent, an antioxidant, deferoxamine, feroxamine, a tin complex, a tin porphyrin complex, a metal chelator, ethylenediaminetetraacetic acid (EDTA), an EDTA-Fe complex, dimercapto succinic acid (DMSA), 2,3-dimercapto-1-propanesulfonic acid (DMPS), penicillamine, minocycline, prednisone, azathioprine, mycophenolate mofetil, mycophemolic acid, sirolimius, cyclorsporine, or tacrolimusan antibiotic, an iron chelator, a porphyrin, hemin, vitamin B 12, an Nrf2 pathway activator, bardoxolone, ACE inhibitors, enalapril, glycine polymers, antioxidants, glutathione, N acetyl cysteine, a chemotherapeutic, QPI-1002, QM56, SVT016426 (QM31), 16/86 (third generation ferrostatin), BASP siRNA, CCX140, BIIB023, CXA-10, alkaline phosphatase, Dnmtl inhibitor, THR-184, lithium, formoterol, IL-22, EPO, EPO derivative, agents that stimulate erthyropoietin, epoeitn alfa, darbepoietin alfa, PDGF inhibitor, CRMD-001, Atrasentan, Tolvaptan, RWJ-676070, Abatacept, Sotatercept, an anti-infective agent, an antibiotic, an anti-viral agent, an anti-fungal agent, an aminoglycoside, a nonsteroidal anti-inflammatory drug (NSAID), a diuretic drug, a statin, a senolytic, a corticosteroid, a glucocorticoid, a liposome, renin, angiotensin, ACE inhibitor, mediator of apoptosis, mediator of fibrosis, drug that targets p53, Apaf-1 inhibitor, RIPK1 inhibitor, RIPK3 inhibitor, inhibitor of IL17, inhibitor of IL6, inhibitor of IL23, inhibitor of CCR2, nitrated fatty acids, angiotensin blockers, agonists of the ALK3 receptor, and retinoic acid. The therapeutic composition can be administered in combination with a renal protective agent or a renal prophylactic agent, including, but not limited to, thiazide, bemetanide, ethacrynic acid, furosemidem torsemide, glucose, mannitol, amiloride, spironolactone, eplerenone, triamterene, potassium canrenoate, bendroflumethiazide, hydrochlorothiazide, vasopressin, amphotericin B, acetazolamide, tovaptan, conivaptan, dopamine, dorzolamide, bendrolumethiazide, hydrochlorothiazide, caffeine, theophylline, theobromine, a statin, a senolytic, navitoclax obatoclax, a corticosteroid, prednisone, betamethasone, fludrocortisone, deoxycorticosterone, aldosterone, cortisone, hydrocortisone, belcometasone, mometasone, fluticasone, prednisolone, methylprednisolone, triamcinolone acetonide, a glucocorticoid, dexamethasone, a steroid, budesonide, triamcinolone acetonide, an anti-inflammatory agent, an antioxidant, a nonsteroidal anti-inflammatory drug (NSAID), deferoxamine, iron, tin, a metal, a metal chelate, ethylenediaminetetraacetic acid (EDTA), dimercap to succinic acid (DMSA), 2,3-dimercapto-l-propanesulfonic acid (DMPS), penicillamine, an antibiotic, an aminoglycoside, an iron chelator, a porphyrin, an Nrf2 pathway activator, bardoxolone, ACE inhibitors, enalapril, glycine polymers, antioxidants, glutathione, N-acetyl cysteine, a PDGF inhibitor, lithium, ferroptosis inhibitors, vitamin B 12QPI-1002, QM56, SVT016426 (QM31), 16/86 (third generation ferrostatin), BASP siRNA, CCX140, BIIB023, CXA-10, alkaline phosphatase, Dnmtl inhibitor, THR-184, lithium, formoterol, IL-22, EPO, EPO derivative, agents that stimulate erthyropoietin, epoeitn alfa, darbepoietin alfa, PDGF inhibitor, CRMD-001, Atrasentan, Tolvaptan, RWJ-676070, Abatacept, Sotatercept, an anti-infective agent, an antibiotic, an anti-viral agent, an antifungal agent, an aminoglycoside, a nonsteroidal anti-inflammatory drug (NSAID), a diuretic drug, a statin, a senolytic, a corticosteroid, a glucocorticoid, a liposome, renin, angiotensin, ACE inhibitor, mediator of apoptosis, mediator of fibrosis, drug that targets p53, Apaf-1 inhibitor, RIPK1 inhibitor, RIPK3 inhibitor, inhibitor of IL17, inhibitor of IL6, inhibitor of IL23, inhibitor of CCR2, nitrated fatty acids, angiotensin blockers, agonists of the ALK3 receptor, SGLT2 modulator, and/or retinoic acid.

The therapeutic composition can improve one or more markers of kidney function in the subject, such as, for example, those selected from reduced blood urea nitrogen (BUN) in the subject, reduced creatinine in the blood of the subject, improved creatinine clearance in the subject, reduced proteinuria in the subject, reduced albumin: creatinine ratio in the subject, improved glomerular filtration rate in the subject, reduced NAG in the urine of the subject, reduced NGAL in the urine of the subject, reduced KIM-1 in the urine of the subject, reduced IL-18 in the urine of the subject, reduced MCP1 in the urine of the subject, reduced CTGF in the urine of the subject; reduced collagen IV fragments in the urine of the subject; reduced collagen III fragments in the urine of the subject; and reduced podocyte protein levels in the urine of the subject, wherein the podocyte protein is selected from nephrin and podocin, reduced cystatin C protein in the blood of a subject, reduced b-trace protein (BTP) in the blood of a subject, and reduced 2-microglobulin (B2M) in the blood of a subject.

The method of inducing cardiomyocyte regeneration, cardiac repair, vasculogenesis and/or cardiomyocyte differentiation provided herein comprises administering a therapeutic composition to a subject, wherein the therapeutic composition is administrated as a first dose and a second dose separated by an interval.

In some embodiments, in a swine model of cardiac IR injury in young adult 8-12-week-old pigs, combined inhibition of miR-99/100 and let-7a/c was necessary and sufficient to activate an underlying cardiac regeneration process in the hearts when administered IV at a single, relatively low dose immediately after reperfusion. JBT-miR2, unlike control virus, stimulated the regenerative capabilities of cardiomyocytes, increased heart function, and decreased cardiac volumes post administration. Furthermore, scar tissue was reduced after administration and the levels of creatine kinase, a biomarker of muscle injury, as well as circulating levels of blood urea nitrogen levels decreased with JBT-miR2 treatment. Particularly, the therapeutic composition can reduce creatine kinase (CK) and lactate dehydrogenase (LDH) levels, left ventricle (LV) cavity volume, size of scar, blood urea nitrogen (BUN) level, blood urea nitrogen/creatine ratios and anterior wall thickness; and increases LV ejection fraction and LV mass, LV wall displacement/motion, albumin/globulin (A/G) ratios, percentage of lymphocytes, septal and lateral walls thickness compared to a subject not administrated with the therapeutic composition. The creatine kinase (CK) and lactate dehydrogenase (LDH) levels, left ventricle (LV) cavity volume, size of scar, blood urea nitrogen (BUN) level, blood urea nitrogen/creatine ratios and/or anterior wall thickness are reduced by at least 5% (e.g., at least 5%, at least 10%, at least 20%, at least 30%, or at least 50%) compared to the creatine kinase (CK) and lactate dehydrogenase (LDH) levels, left ventricle (LV) cavity volume, size of scar, blood urea nitrogen (BUN) level, blood urea nitrogen/creatine ratios and anterior wall thickness of the subject prior to administration with the therapeutic composition. The LV ejection fraction and LV mass, LV wall displacement/motion, albumin/globulin (A/G) ratios, percentage of lymphocytes, septal and/or lateral walls thickness are increased by at least 5% (e.g., at least 5%, at least 10%, at least 20%, at least 30%, or at least 50%) compared to the LV ejection fraction and LV mass, LV wall displacement/motion, albumin/globulin (A/G) ratios, percentage of lymphocytes, septal and/or lateral walls thickness of the subject prior to administration with the therapeutic composition.

Moreover, the second dose of the therapeutic composition can reduce creatine kinase (CK) and lactate dehydrogenase (LDH) levels, left ventricle (LV) cavity volume, size of scar, blood urea nitrogen (BUN) level, blood urea nitrogen/creatine ratios and anterior wall thickness; and increases LV ejection fraction and LV mass, LV wall displacement/motion, albumin/globulin (A/G) ratios, percentage of lymphocytes, septal and lateral walls thickness compared to a subject not administrated with the second dose of the therapeutic composition. The creatine kinase (CK) and lactate dehydrogenase (LDH) levels, left ventricle (LV) cavity volume, size of scar, blood urea nitrogen (BUN) level, blood urea nitrogen/creatine ratios and/or anterior wall thickness are reduced by at least 5% (e.g., at least 5%, at least 10%, at least 20%, at least 30%, or at least 50%) compared to the creatine kinase (CK) and lactate dehydrogenase (LDH) levels, left ventricle (LV) cavity volume, size of scar, blood urea nitrogen (BUN) level, blood urea nitrogen/creatine ratios and anterior wall thickness of the subject prior to administration of the second dose of the therapeutic composition. The LV ejection fraction and LV mass, LV wall displacement/motion, albumin/globulin (A/G) ratios, percentage of lymphocytes, septal and/or lateral walls thickness are increased by at least 5% (e.g., at least 5%, at least 10%, at least 20%, at least 30%, or at least 50%) compared to the LV ejection fraction and LV mass, LV wall displacement/motion, albumin/globulin (A/G) ratios, percentage of lymphocytes, septal and/or lateral walls thickness of the subject prior to administration of the second dose of the therapeutic composition.

Importantly, no abnormal tissue pathological changes nor abnormal readings in metabolic blood function were found, demonstrating that JBT-miR2 has an acceptable safety profile. JBT-miR2 also demonstrated similar efficacy and safety in middle aged mini pigs as in young adult pigs. The therapeutic composition does not cause arrhythmias, a reduction in a body weight of the subject, liver toxicity, detection of red blood cells in urinalysis, and deviation of hematology values, prothrombin time (PT), activated partial thromboplastin time (APTT), peripheral oxygen saturation (SpO₂), body temperature end-tidal (ET) CO₂ and CO₂ reduction reaction (RR) from clinical reference ranges. A lack of liver toxicity can be measured by the level of liver enzymes selected from alanine transferase levels (ALT), alkaline phosphatase levels (ALP), aspartate aminotransferase levels (AST), total bilirubin levels (TBIL), and direct bilirubin levels (DBIL). The hematology values can include percentages of reticulocytes, monocytes, eosinophils, and basophils; counts of platelet, red blood cell, lymphocytes, monocytes, eosinophils, and basophils; and size and shape of erythrocytes and red blood cells.

In vivo AAV delivery of inhibitors of these miRs into the hearts of pigs with left coronary artery ligation increased the expression of FNTP and SMARCA5. Cardiac regeneration was achieved, as indicated by the expression of proliferation and cytokinesis markers, labeled uridine incorporation into DNA, together with scar tissue regression and heart functional improvement.

In some embodiments, two doses of IC and IV administered JBT-miR2-ADD that delivers inhibitors to miR-99, miR-100, let-7a and let-7c is necessary and sufficient to re-activate an underlying cardiac regeneration process in hearts. In some embodiments, the hearts are pig hearts with IR injury. In some embodiments, repeat dosing of JBT-miR2-ADD is safe, with no liver toxicity or arrhythmia up to 10-wks post-IR. In some embodiments, JBT-miR2 reduces scar in the left ventricular (LV). Thus, the JBT reprograming approach (e.g., JBT-miR2) can largely reduce death from IHD induced HF.

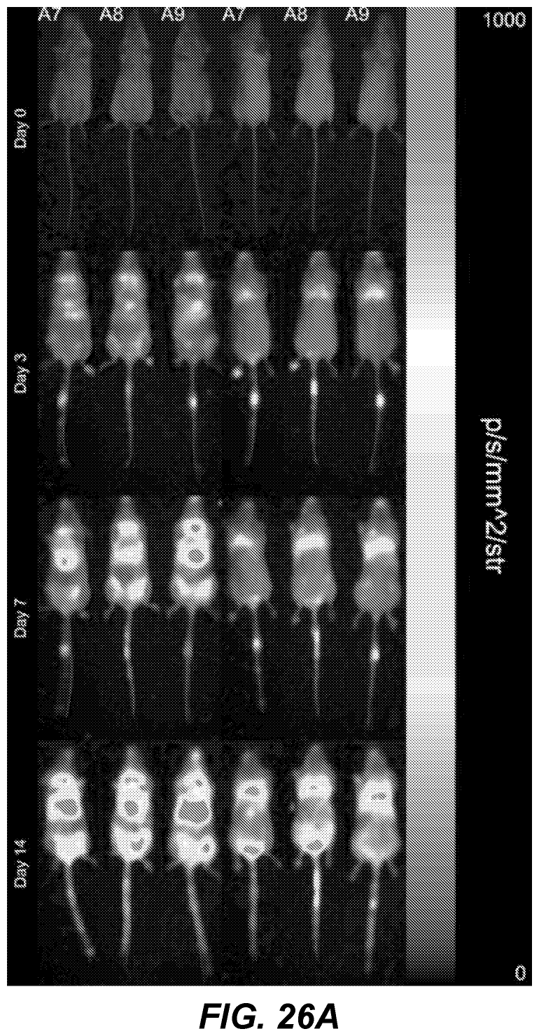

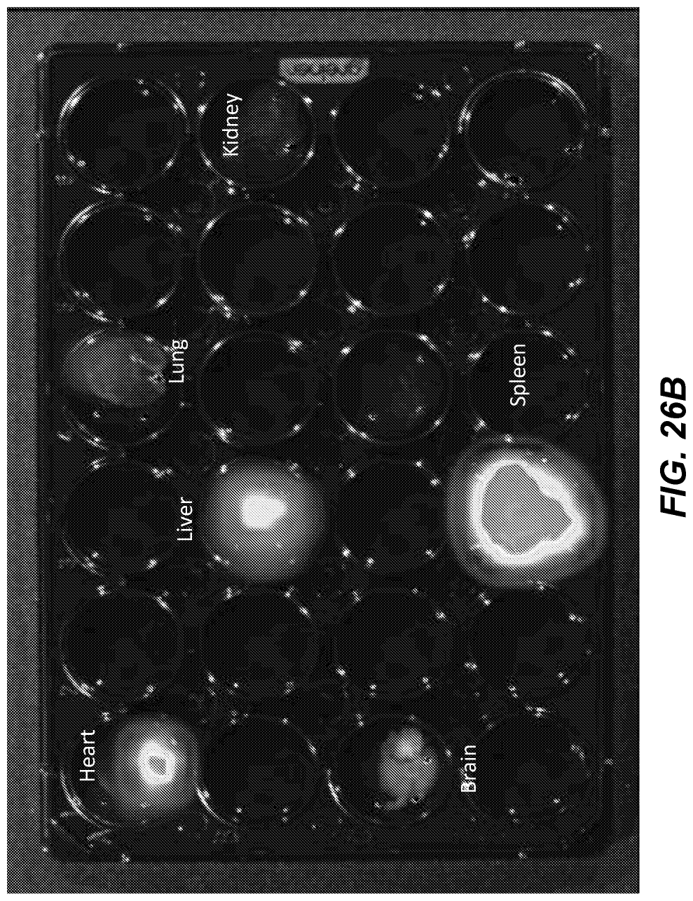

A significant problem in developing cardiovascular microRNA therapeutics is to target the organ effectively to get a therapeutic effect. In some embodiments, JBT-miR2 has tropism of the heart. As shown in the whole body bioluminescence imaging of mice (FIG. 26), JBT-miR2 inhibited miR-99/100 and Let-7a/c and targeted muscle and liver. The virus containing JBT-miR2 was safe and tolerable at 30x the effective dose. The viral particles containing JBT-miR2 were well tolerated amongst all animal models with no adverse signs or symptoms, including during necropsy (data not shown).

### Combination Therapies

In some embodiments, the therapeutic compositions and pharmaceutical formulations including the microRNA antagonists disclosed herein, such as those provided in the Sequence Listing, or those including a combination of the microRNA antagonists disclosed herein, or an expression vectors comprising a nucleotide sequence encoding one or more microRNA antagonists disclosed herein, can be used in combination with one or more additional therapeutic agents. In some embodiments, the therapeutic compositions and pharmaceutical formulations including the microRNA antagonists disclosed herein, such as those provided in the Sequence Listing, or those including a combination of the microRNA antagonists disclosed herein, or an expression vectors comprising a nucleotide sequence encoding one or more microRNA antagonists disclosed herein, can be used in combination with one or more therapeutic therapies.

Generally, any therapeutic approach pharmacological or non-pharmacological for muscular dystrophies can be suitably employed as additional therapeutic agents and therapies in the methods disclosed herein. Examples of additional therapeutic agents and therapies that can be used in combination with the microRNA antagonists disclosed herein, or a composition or formulation that include a combination of the microRNA antagonists disclosed herein, or an expression cassette comprising a nucleotide sequence encoding one or more microRNA antagonists disclosed herein, or a vector comprising one or more of such expression cassettes, include, but are not limited to, Idebenone, Eplerenone, VECTTOR, AVI-4658, Ataluren/PTC124/Translarna, BMN044/PR0044, CAT-1004, any gene therapy for MD including MicroDystrophin AAV gene therapy (SGT-001), Galectin-1 therapy (SB-002), LTBB4 (SB-001), rAAV2.5-CMV-minidystrophin, glutamine, NFKB inhibitors, sarcoglycan, delta (35 kDa dystrophin-associated glycoprotein), insulin like growth factor-1 (IGF-1) expression, genome editing through the CRISPR/Cas9 system, any gene delivery therapy aimed at reintroducing a functional recombinant version of the dystrophin gene, Exon skipping therapeutics, read-through strategies for nonsense mutations, cell-based therapies, utrophin upregulation, myostatin inhibition, anti-inflammatories/anti-oxidants, mechanical support devices, any standard therapy for muscular dystrophy, and combinations thereof.

Additional therapeutic agents useful for the methods of the present disclosure also include, but are not limited to, anti-platelet therapy, thrombolysis, primary angioplasty, Heparin, magnesium sulphate, Insulin, aspirin, cholesterol lowering drugs, angiotensin-receptor blockers (ARBs) and angiotensin-converting enzyme (ACE) inhibitors. In particular, ACE inhibitors have clear benefits when used to treat patients with chronic heart failure and high-risk acute myocardial infarction; this is possibly because they inhibit production of inflammatory cytokines by angiotensin II. A non-limiting listing of additional therapeutic agents and therapies includes ACE inhibitors, such as Captopril, Enalapril, Lisinopril, or Quinapril; Angiotensin II receptor blockers, such as Valsartan; Beta-blockers, such as Carvedilol, Metoprolol, and bisoprolol; Vasodilators (via NO), such as Hydralazine, Isosorbide dinitrate, and Isosorbide mononitrate; Statins, such as Simvastatin, Atrovastatin, Fluvastatin, Lovastatin, Rosuvastatin or pravastatin; Anticoagulation drugs, such as Aspirin, Warfarin, or Heparin; or Inotropic agents, such as Dobutamine, Dopamine, Milrinone, Amrinone, Nitroprusside, Nitroglycerin, or nesiritide; Cardiac Glycosides, such as Digoxin; Antiarrhythmic agents, such as Calcium channel blockers, for example, Verapamil and Diltiazem or Class III antiarrhythmic agents, for example, Amiodarone, Sotalol or, defetilide; Diuretics, such as Loop diuretics, for example, Furosemide, Bumetanide, or Torsemide, Thiazide diuretics, for example, hydrochlorothiazide, Aldosterone antagonists, for example, Spironolactone or eplerenone. Alternatively or in addition, other treatments of cardiac disease are also suitable, such as Pacemakers, Defibrillators, Mechanical circulatory support, such as Counterpulsation devices (intraaortic balloon pump or noninvasive counterpulsation), Cardiopulmonary assist devices, or Left ventricular assist devices; Surgery, such as cardiac transplantation, heart-lung transplantation, or heart-kidney transplantation; or immunosuppressive agents, such as Myocophnolate mofetil, Azathiorine, Cyclosporine, Sirolimus, Tacrolimus, Corticosteroids Antithymocyte globulin, for example, Thymoglobulin or ATGAM, OKT3, IL-2 receptor antibodies, for example, Basilliximab or Daclizumab are also suitable.

In some embodiments, at least one of the additional therapeutic agents or therapies includes a biologic drug. In some embodiments, the at least one additional therapeutic agent or therapy comprises a gene therapy or therapeutic gene modulation agent. As used herein, therapeutic gene modulation refers to the practice of altering the expression of a gene at one of various stages, with a view to alleviate some form of ailment. It differs from gene therapy in that gene modulation seeks to alter the expression of an endogenous gene, for example through the introduction of a gene encoding a novel modulatory protein, whereas gene therapy concerns the introduction of a gene whose product aids the recipient directly. Modulation of gene expression can be mediated at the level of transcription by DNA-binding agents, which can be for example, artificial transcription factors, small molecules, or synthetic oligonucleotides. Alternatively or in addition, it can also be mediated post-transcriptionally through RNA interference.

The therapeutic compositions, pharmaceutical formulations disclosed herein and the additional therapeutic agents or therapies can be further formulated into final pharmaceutical preparations suitable for specific intended uses. In some embodiments, the therapeutic composition and the additional therapeutic agent or therapy are administered in a single formulation. In some embodiments, each of the therapeutic composition and the additional therapeutic agent or therapy is administered in a separate formulation. In some embodiments of the methods disclosed herein, the therapeutic composition and/or the additional therapeutic agent or therapy is administered to the subject in a single dose. In some embodiments, the therapeutic composition and/or the additional therapeutic agent or therapy is administered to the subject in multiple dosages. In some embodiments, the dosages are equal to one another. In some embodiments, the dosages are different from one another. In some embodiments, the therapeutic composition and/or the additional therapeutic agent or therapy is administered to the subject in gradually increasing dosages over time. In some embodiments, the therapeutic composition and/or the additional therapeutic agent or therapy is administered in gradually decreasing dosages over time.

The order of the administration of the therapeutic compositions and pharmaceutical formulations, with one or more additional therapeutic agent or therapy, can vary. In some embodiments, a therapeutic composition or pharmaceutical formulation disclosed herein can be administered prior to the administration of all additional therapeutic agent or therapy. In some embodiments, a therapeutic composition or pharmaceutical formulation disclosed herein can be administered prior to at least one additional therapeutic agent or therapy. In some embodiment, a therapeutic composition or pharmaceutical formulation disclosed herein can be administered concomitantly with one or more additional therapeutic agent or therapy. In yet still other embodiments, a therapeutic composition or pharmaceutical formulation disclosed herein can be administered subsequent to the administration of at least one additional therapeutic agent or therapy. In some embodiments, a therapeutic composition or pharmaceutical formulation disclosed herein can be administered subsequent to the administration of all additional therapeutic agent or therapy. In yet some embodiments, a therapeutic composition or pharmaceutical formulation disclosed herein and at least one additional therapeutic agent or therapy are administered in rotation (e.g., cycling therapy). For examples, in some embodiments, a therapeutic composition or pharmaceutical formulation disclosed herein and at least one additional therapeutic agent or therapy are cyclically administered to a subject. Cycling therapy involves the administration of a first active agent or therapy for a period of time, followed by the administration of a second active agent or therapy for a period of time and repeating this sequential administration. Cycling therapy can reduce the development of resistance to one or more therapies, avoid or reduce the side effects of one or more therapies, and/or improve the efficacy of treatment.

In some embodiments, intermittent therapy is an alternative to continuous therapy. For example, intermittent therapy can be used for a period of 6 months on, followed by a period of 6 months off In some embodiments, one or more therapeutic agents or therapies are provided for one month on, followed by one month off. In some embodiments, one or more therapeutic agents or therapies are provided for three months on, followed by three months off.

Accordingly, one or more of the therapeutic compositions or pharmaceutical formulations disclosed herein can be provided before, during and/or after administering one or more additional therapeutic agents or therapies, as described above.

### EXAMPLES

Some aspects of the embodiments discussed above are disclosed in further detail in the following examples, which are not in any way intended to limit the scope of the present disclosure.

### Materials and Methods

The following experimental materials and methods were used for Examples 1-3 described below.

### Overview of Study and Procedures

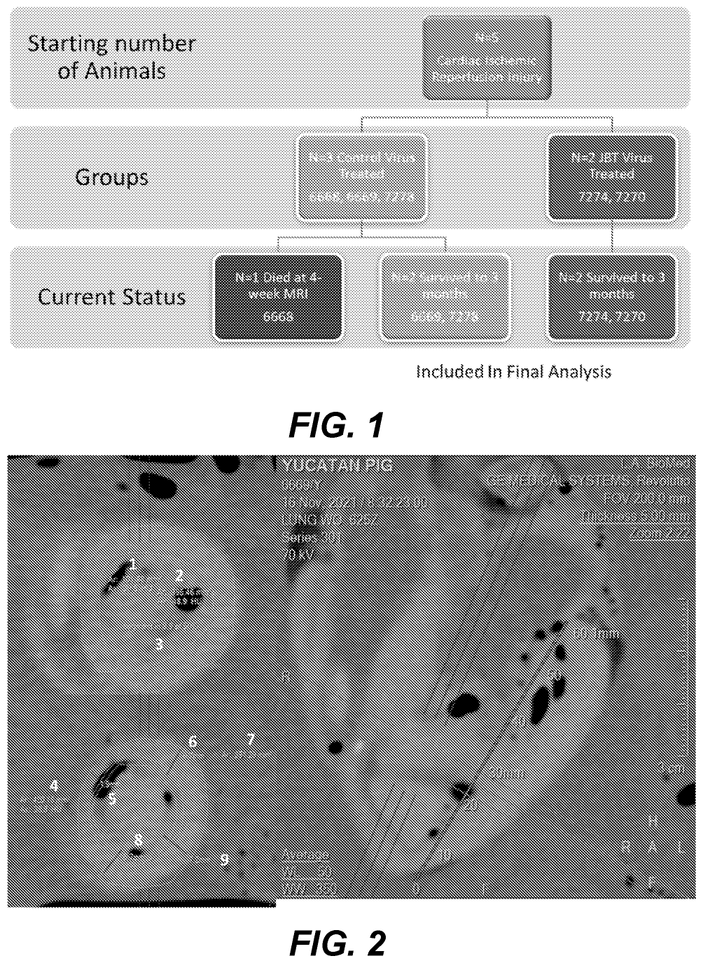

Ischemia was achieved in five 4-6-month-old 20 kg female Yucatan pigs by percutaneous transluminal coronary angioplasty (PTCA) balloon inflation for 60 min in the mid-LAD artery distal to the second diagonal branch. JBT-miR2-ADD (N=2, #7270 and 7274) or control (N=3, #6668, 6669, 7278) was administered twice to the animals (FIG. 1), once at a dose of 2.5×10¹² vg/kg [swine dose calculated from the effective dose of 1×10¹¹ vg/mouse for a 40 g CD-1 mouse (1 kg=1000 g/40 g=25; 25×1×10¹¹ vg=2.5×10¹² vg/kg)] via a 3 mL intracardiac (IC) infusion at a rate of 1/mL/min at reperfusion followed by second IV bolus injection at the same dose after the 8-wk (Day 60) MRI. For the second IV injection, #7270 and 7274 received JBT-miR2-ADD, and #6669, and 7278 received sterile saline.

For patients presenting with an MI, the ease of delivery following catheter intervention to re-establish coronary flow makes IC injection appealing as it allows for selective delivery of therapeutics to the myocardial area of interest and theoretically limits risks of systemic toxicity. However, the therapeutic could have broader application to patients if administered by IV administration and in the chronic heart failure (CHF) setting where percutaneous coronary intervention (PCI) is not possible.

The IV technique, through a peripheral or central venous catheter, is the simplest delivery mode and avoids the risk of an invasive procedure. The animals underwent the procedures shown in Table 1. The following protocol changes were made, confirming that: 1) a repeat IV dose at 8-wks was efficacious and safe. IV administration was possible so that the virus could improve heart function in HF reduced EF (HFrEF) applicable to the IHD population. Others have demonstrated that a repeat dose of AAV2/9 does not instigate an immune response after one-month in non-human primates. 2) Follow-up was extended from 8-wks (Day 60) to 10-wks (Day 80) to assess the efficacy and safety of the second dose. 3) Cardiac CT was used to quantify scar size in post-mortem hearts.

TABLE 2
SCHEDULE OF PROCEDURES PHASE 1 SWINE STUDIES

| | | | | Ischemia Reperfusion | | | | | | | | Follow | |
|---|---|---|---|---|---|---|---|---|---|---|---|---|---|
| Assessments | Screening | | | Surgery | | | | | | | | up | |
| Day | Day −7 to 0 | 1 | 1 | 1 | 1 | 1 | 1 | 1 | 1 | 2 | 30 | 60 | 80 |
| Weeks | | | | | | | | | | | 4 | 8 | 10 |
| Time (minutes) | | 120 | 0 | 5 | 10 | 30 | 60 | 90 | 120 | | | | |
| Weight kg | X | | | | | | | | | | X | X | X |
| Vital signs: BP, HR Resp rate, | X | | X | | | | X | | X | X | X | X | X |
| Temp | | | | | | | | | | | | | |
| Physical examination | X | | X | | | | | | X | X | X | X | X |
| Virus administration | | | | | | | X | | | | | X | |
| PK Viral concentration | | | X | | | | X (65') | X | X | | X | X | X |
| Cardiac Magnetic Resonance | X | | | | | | | | | | X | X | X |
| Imaging | | | | | | | | | | | | | |
| Fluoroscopy C-Arm | | X | X | X | X | X | X | X | X | | | | |
| Intracardiac ECHO (ICE) | | X | | | | X | X | | | | | | |
| Electrocardiograms | X | X | X | X | X | X | X | X | X | | X | X | X |
| AAV2/9 N-ab titers # | | X | | | | | | | | | X | X | X |
| Metabolic blood function | | X | | | | | | | X | | X | X | X |
| tests + LDH | | | | | | | | | | | | | |
| Hematology | X | | | | | | | | X | | X | X | X |
| Coagulation test | | X | | | | | | | X | | X | X | X |
| Urinalysis | X | | | | | | | | | | X | X | X |
| Medications daily | X | X | | | | | | | | X | X | X | X |
| Cardiac CT | | | | | | | | | | | | | X |
| Tissue Collection/Pathology | | | | | | | | | | | | | X |
| Adverse events (AEs) daily | X | X | | | | | | | | X | X | X | X |

In Table 2: #, collected but not analyzed; ICE, Intracardiac Echo; LDH, lactate dehydrogenase; AAV2/9 N-ab titers, AAV2/9 neutralizing antibody titers. Animals

The animals were checked carefully for preexisting diseases during acclimatization 3 days before undergoing any procedure. They were maintained at an atmospheric temperature of 68-72° F. and a humidity of 30%-70% with ad libitum access to water and fed a commercial diet (Teklad 8753). This was a pilot study to reduce the number of pigs in the experiment to about N=3/Grp. Female animals were used.

### LAD Occlusion

Fasted animals (o/n) were weighed 24 h before surgery for their dosing weight. Surgical anesthesia was continuously monitored as follows: Joint tone, movement, BP and HR, vital signs, and ERG. Sedation was achieved with 4.4 mg/kg Telazol, 2.2 mg/kg ketamine and 2.2 mg/kg xylazine intra-muscular as a cocktail, and surgical anesthesia was maintained with isoflurane 1.5-2.5%. Ventilation with 100% O₂ was provided with a ventilator and maintained partial pressure of carbon dioxide (PCO2) at 35 mmHg. Animals also received 0.02-0.04 mg/kg buprenorphine and 4 mg/kg carprofen intra-muscular pre-operatively to prevent wind up pain. Prophylactic antibiotics were administered pre-operatively, exceeding 5 mg/kg IM. An isotonic saline drip was administered via a peripheral venous line (300 ml/hr) to prevent dehydration. Animals were placed in the supine position. 12 lead EKG (Philips/HP Page writer XLI; Philips Healthcare, Andover, MA) and blood gas levels were measured before LAD occlusion. Body temperature was kept at 36.0° C.-37.2° C. with a heating pad and a Bair Hugger system. EKG leads were attached to the limbs and cardiac electrical signals were monitored on a Lifepak 12 monitor/defibrillator and a PowerLab data acquisition system (ADInstruments, CO) for offline EKG analysis (LabChart ADInstruments). EKG analysis pre-settings for swine were: QRS width 40 ms, R—R waves 200 ms, Pre-P baseline 50 ms, Max PR 140 ms, Max RT 400 ms, and ST height 60 ms from alignment.

### ICE Imaging

The femoral vein access was obtained after ultrasound visualization. A J-wire 0.035 was introduced into the femoral vein and into the inferior vena cava. A 9 Fr sheath was placed in the femoral vein over the wire. The wire was removed and intracardiac ultrasound, 8 Fr Siemens catheter was introduced through the 9 Fr sheath and navigated through the IVC to the right atrium. The catheter was repositioned until the image of the LV was obtained. Definity was given IV. visualization of the walls of the LV, including the apex, were recorded. LAD occlusion was confirmed by contrast injection and ST segment elevation on the EKG. An ICE image was acquired 30 mins from the onset of ischemia to confirm regional wall motion abnormality. The same imaging was repeated after balloon occlusion (induction of MI). Catheters were withdrawn at the end of the procedure. After 60 mins of LAD occlusion, the PTCA balloon was deflated. The animals were recovered.

### MRI

ECHO is commonly used in experimental animals and man because of ease of use but is limited by planographic views that do not reflect the full extent of myocardial damage. In contrast, 3D MRI provides accurate measurement of cardiac volumes, function, and mass. Anesthetized and intubated pigs were placed in a supine position with the heart at the isocenter of the MRI unit and an 8-channel cardiac surface array coil was placed over the heart. Anesthesia was maintained with isoflurane 1.5-2.5% with 100% O₂ using a ventilator and a PCO2 of 35 mmHg throughout the procedure. MRI images were acquired with a GE Discovery MR750 3.0 T scanner, using a breath-hold peripheral ECG gated, multi-NEX steady-state free precession pulse sequence (FIESTA). The heart was scanned along two long axis views (vertical and horizontal) and with a set of short axis views covering the entire LV from atrioventricular valve plane to the apex. The following parameters were used: isotropic field of view 30 cm, slice thickness 4-4.5 mm, no gap between each slice, minimum repetition time (˜3.69-4.32 msec), minimum echo time (˜1.62-1.97 msec), flip angle 49°, 20 phases, acquisition matrix 224×224, reconstruction matrix 512×512, NEX 2. LV size/EF: Image segmentation was performed on the end-systole (ED) and end-diastole (ES) phases using Seg3D 2 (University of Utah, NIH/NIGMS Center for Integrative Biomedical Computing) to best estimate LV endocardial borders (excluding papillary muscles) at ED and ES from the short-axis MR images of the heart. For 3D reconstruction wall motion analysis and size of the ischemic area, the borders derived from the images were imported into Continuity (6.4b revision 6734, National Biomedical Computation Resource). The simplest normalization for ventricular size was to calculate the ratio of displacement at each node to the calculated EDSA. Matlab® (MathWorks, MA) and Continuity Software were used for analyses. MRI endpoints included Interventricular septal thickness, Interventricular posterior wall, LV mid-ventricular area, LV mass, EDV, ESV, EF, LV outflow tract velocity (LV OTV) time integral, Global Longitudinal Strain, LV diastolic function, LV wall motion score. LVEF was obtained by measuring EDV and ESV by Simpson's biplane method in apical 4 and apical 2 chambers. EDV and ESV were also calculated using the biplane area-length method for an ellipsoidal model of the LV. Stroke volume (SV) was obtained by taking the EDV−ESV. Cardiac output (CO) was calculated by taking the SV×the HR. LV VTI was measured at the LVOT.

### Study Drug

Two batches of endotoxin-free JBT-miR2-ADD and control at a titer of at least 3×10¹⁴ vg were manufactured at non-GMP and at in vivo grade by Vigene Biosciences Inc. (now Charles River) using suspension cell lines. This vendor has GMP capability for human use. The identity of the virus was confirmed using PCR using primers to the ITR sequences.

Drug potency between batches was required to be consistent. pMIR-REPORT™ miRNA Reporter Expression vectors (AM5795, Applied Biosystems) have complementary binding sites for four miRs (LUC 1 (miR-99), LUC 2 (miR-100), LUC 3 (let-7a) and LUC 4 (let-7c)) cloned into the luciferase reporter gene. Human induced pluripotent stem cells (IPSCs) derived cardiac myocytes (CMs) and media were purchased from Ncardia, PA, pre-plated on 96 well plates according to the manufacturer's instructions. Each well of cells were transfected with 50 ng of pMIR-REPORT™ DNA and 10 ng of s-gal plasmid using Lipofectamine® 2000 Reagent (Life Technologies, CA). 24 h after transfection, the cells were treated with increasing concentrations of JBT-miR2-ADD in triplicate for 24 h in Ax-M-BMCC250 media followed by culturing the cells in Ax-M-HC250 media for 48 h. The cells were then harvested for Luciferase and R-gal activity using One Glo Luc (E6110, Promega), Beta Glo Luc (E4720, Promega), Glo Lysis Buffer (E2661, Promega) and BioTec Synergy HT Part Number BTSIAFRT/SN 235106.

Luciferase activity was normalized to R-gal activity and expressed as significant fold activation over the LUC plasmids alone. Dose dependent increase in luciferase activity should be evident with JBT-miR2-ADD infection. The effective dose of 2.5×10¹² vg/kg of JBT-miR2-ADD or control was diluted in a final volume of 3 mL in sterile saline and administered IC at reperfusion and then as a 1 mL IV bolus in an ear vein at 4-, 8- and 12-wks post IR.

### LV Scar

Under deep (5%) isoflurane anesthesia, the heart was arrested in diastole with a saturated solution of 30-90 mEq KCL injected through the jugular vein, excised, and analyzed for cardiac CT quantification of scar, cardiac volumes, and mass.

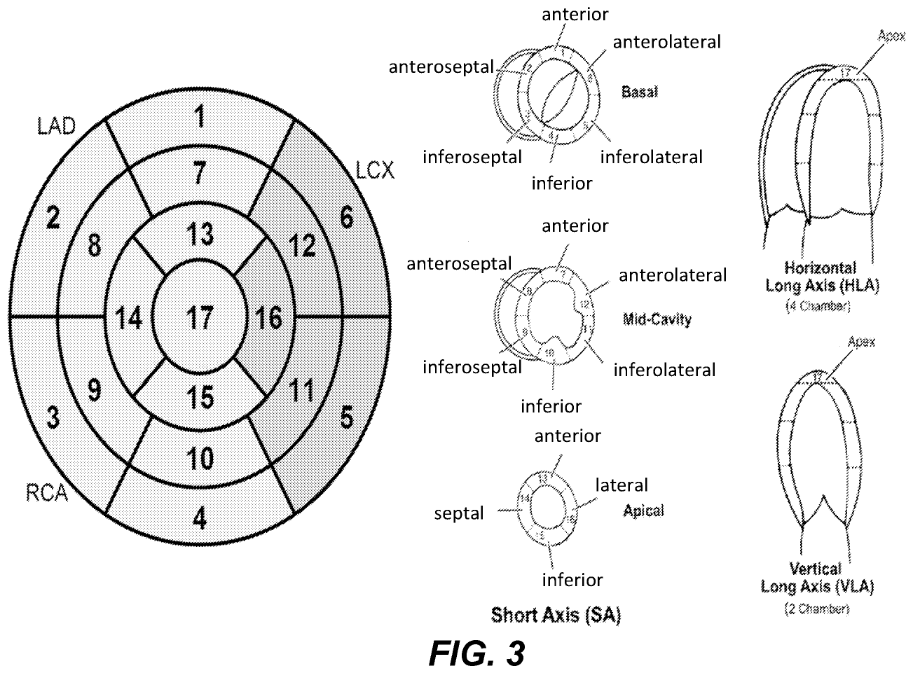

A CT scanner GE 256 row detectors, 70 kVp, 500 mA, with image spatial resolution of 0.625 mm/per slice was used to image the hearts that were submerged in ice cold water. The parameters determined included: (1) LV mass, LV wall thickness and Hounsfield Units (HU) measure in the short axial image from base to apex, sum or average of all measures (FIG. 2); (2) The characteristics of the ischemia area are 1. Thinner in local LV wall, 2. Mild decrease in CTHU, 3. Mild bulging as shown in FIG. 3. The 17 segments of Echo shown in FIG. 3 were used to locate the diseased area; (3) Definition of low CTHU area: by vision, the objects with 4-5 CTHU differences can be distinguished. In some examples, it was measured in interest area with a difference >5 HU and with the thinner wall in comparing surrounding area. After CT of the hearts, the hearts were fixed in 10% NBF (4 days) and digitally photographed in color.

Some errors can exist due to that the contrast between the LV and RV, between the LV cavity and mass was not optimal. All measures were done manually. Finally, the area with a low CTHU was under-estimation than true value since the thickness decreased compared to the normal areas.

### Safety of JBT-miR2-ADD

The primary goals of preclinical safety evaluation are: (1) To identify an initial safe and effective dose and subsequent dose escalation schemes in humans; (2) to identify potential target organs for toxicity and for the study of whether such toxicity is reversible; and (3) to identify safety parameters for clinical monitoring. The route and frequency of administration should be as close as possible to that proposed for clinical use.

Safety parameters between JBT-miR2-ADD treated and control animals were compared for 1) clinical chemistry, coagulation, urinalysis, and hematology, 2) arrhythmogenesis, 3) pathology on tissues from major organs, 4) vital signs and 5) adverse events (AEs). The normal values are shown in Table 3 below.

TABLE 3
Quality Vet Lab-Porcine-Yucatan (Yucatan Pigs) Reference Ranges

| Hematology | N = 161 | | | Chemistry | N = 153 | |
|---|---|---|---|---|---|---|
| Siemens-advia 120 | | | | D × C 700AU | | |
| | | Reference | | | | Reference |
| Test | Units | Range | | Test | Units | Range |
| WBC | × 10³/μL | 6-23 | | ALB | g/dL | 3.1-4.2 |
| RBC | × 10⁶/μL | 4.9-8.0 | | TP | g/dL | 5.5-7.5 |
| HGB | g/dL | 9-16 | | ALP | U/L | 22-164 |
| HCT | % | 28-46 | | ALT | U/L | 19-76 |
| MCV | fL | 50-64 | | AST | U/L | 7-50 |
| MCH | pg | 16-22 | | CPK** | U/L | 131-447 |
| MCHC | g/dL | 31-35 | | TBIL | mg/dL | 0.0-0.3 |
| RDW | % | 12-21 | | DBIL | mg/dL | 0.0-0.1 |
| PLT | × 10³/μL | 174-745 | | BUN | mg/dL | 9.0-21.0 |
| MPV | fL | 4-13 | | CREAT | mg/dL | 0.4-1.2 |
| % Neut | % | 21-73 | | CA | mg/dL | 9.2-10.7 |
| % Lymph | % | 23-71 | | CHOL | mg/dL | 59-106 |
| % Mono | % | 1-6 | | GLU | mg/dL | 43-128 |
| % Eosin | % | 0-6 | | PHOS | mg/dL | 5.7-8.8 |
| % Baso | % | 0-2 | | CO2 | mEq/L | 22-34 |
| % Bands | % | 0 | | NA | mEq/L | 133-145 |
| #Neut | × 10³/μL | 0-14 | | K | mEq/L | 3.1-5.1 |
| #Lymph | × 10³/μL | 2-11 | | CI | mEq/L | 95-107 |
| #Mono | × 10³/μL | 0-1 | | GLOB | g/dL | 1.9-3.6 |
| #Eosin | × 10³/μL | 0-1 | | A/G | Ratio | 0.9-1.8 |
| #Baso | × 10³/μL | 0-1 | | B/C | Ratio | 6-34 |
| #Bands | × 10³/μL | 0-1 | | IBIL | mg/dL | 0.0-0.2 |
| % RETIC | % | 0-3 | | ANION | mEq/L | 7-22 |
| # RETIC | × 10⁹/μL | 0-177 | | Chemistry range based on Mean+/−2 SD | | |
| Hematology Reference Range = Mean+/−2 SD | | | | Except for ** = Mean+/−1 SD | | |
| Coagulation | | Stago STA Compact | | | | |
| PT | seconds | 11.8-13.8 | N = 58 | GGT U/L | 22-64 | N = 29 |
| APTT | seconds | 11.5-17.2 | N = 58 | SDH U/L | 0-4 | N = 29 |

Coagulation range based on Mean+/−2 SD

For clinical chemistry, 2 mL of blood was collected in BD Vacutainer Serum 367812 tubes for measures of ALP CK, TP, TCO2, Na, I-BIL, ALT, CA, PHOS, D-BIL, K, GLOB, AST, ALB, CREAT, T-BIL, Cl, ALB/GLOB, CHOL, GLU, BUN, Anion Bun/Creat Ratio. For coagulation, 2 mL of blood was collected in 3.2 00 Sodium citrate tubes for measures of prothrombin time (PT), activated partial thromboplastin time (APTT), Fibrinogen. For urinalysis, 2 mL of urine was collected in BD Vacutainer Serum 367812 tubes for physical exam of color, clarity, volume, and specific gravity; and chemical exam of dipstick, ictotest, protein by sulfosalicylic acid, and microscopic examination.

Blood (2 mL) for hematology was collected in K2EDTA Tubes for automated measures of Blood Count (e.g., wbc, rbc, hgb, hct, mcv, inch, mchc, rdw, mpv, Differential, red blood cell (RBC) morphology, Platelet Count), with all slides reviewed automatedly by a Technologist, Retic. Both percent and absolute values were measured and calculated.

The samples were taken at 4-, 8-, 12- and 16-wks post-IR (prior to re-dosing). Raw values and abnormalities were reported by the testing laboratory, QVL. Values were averaged for JBT-miR2-ADD treated vs. control at each time point and compared.

Arrhythmogenesis were determined by EKG in JBT-miR2-ADD, and control treated animals at baseline, during IR surgery, 4-, 8-, 12- and 16-wks post-IR (prior to re-dosing and after immediately after re-dosing) using 12-lead EKG parameters (HR, PR, QRS, QT, QTcF) and reported as mean averages from duplicate recordings.

For histology, at necropsy, tissues were fixed for 24 h in 10% (v/v) NBF and embedded in paraffin for sectioning. Non-cardiac tissue (brain, lungs, liver, spleen, kidney, skin and skeletal muscle) was stained with H&E for toxicity including oncogenesis markers such as cytokeratin for neoplasms of epithelial cell lineage, vimentin for neoplasms of mesenchymal cell lineage (sarcomas and melanomas), or CD45 for neoplasms of hematopoietic cell lineage. Pathology assessment of toxicological changes was determined using the General Approach to Histopathologic Evaluation.

Vital signs were recorded at baseline, before and after virus administration and at 4-, 8-, and 10-wks post-IR (prior to re-dosing and after re-dosing) and include BP, temperature, HR, respiratory rate. AEs were recorded throughout the experiment period and defined as a medication addition/treatment, injury, or death at any time post study drug administration.

### Pharmacokinetic Assay

A qPCR assay was developed to provide pharmacokinetic (PK) data (e.g., circulating and in vivo tissue levels of virus) using primers to the TUD1 decoy inhibitor in JBT-miR2-ADD with the ability to detect levels as low at 8 vg/mL in whole blood from pigs. Viral DNA was extracted from 100 μl of blood using the PureLink Viral RNA/DNA Mini kit, Invitrogen. Similarly, viral DNA was extracted from control swine serum spiked with known virus concentrations from up to 1×10¹² vg/ml to generate a standard curve. QPCR of TUD1 was conducted using the Stratagene Mx3000p™ qPCR machine using the Forward primer: CCTAGTAGAACATGGGCATCTG (SEQ ID NO: 7), Reverse Primer: GCGATCCTAGTAGTTGGTGTTC (SEQ ID NO: 8) and probe: AGACACTGGTCTTATGAACATGGGCA (SEQ ID NO: 9). The PCR reaction for all samples was: TaqMan Master Mix (2×) 10 μl, TaqMan TUD1 (10 μM) 1 μl, Blood RT products 1 μl, ddH₂O 8 μl, 95° C. 10 min, (95° C. 15 sec, 60° C. 1 min)×40 cycles. An equation was derived from the line of the Standard Curve ΔCt Values of increasing JBT-miR2-ADD levels minus blank and was used to determine unknown levels of JBT-miR2-ADD in swine blood. PK parameters were measured using GraphPad Prism.

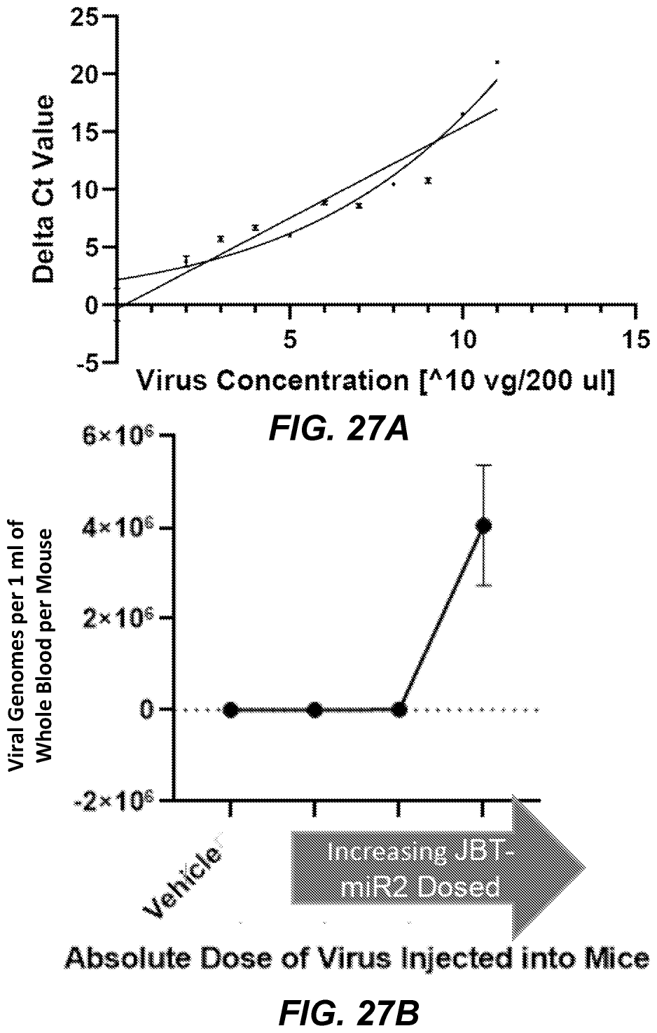

A similar QPCR PK assay was developed for JBT-miR TuDS in mice. The Standard curve delta Ct values of increasing JBT-miR2 levels in mouse serum minus blank is shown in FIG. 27A. From the standard curve, the formula below for calculating absolute dose of virus in mice was obtained. The change of viral genomes per 1 mL whole blood of mice with the increasing JBT-miR2 dose is shown in FIG. 27B.

$$x = \frac{1 {0}^{( y + 0.3615 )}}{1.574} \qquad ( 1 )$$

### Data Analysis & Statistics

Statistical analysis was done by individuals who are unaware of treatment allocation and expressed as Mean±SD using Prism 5.0 (GraphPad Software, Inc., SD). Efficacy and safety parameters of JBT-miR2-ADD were compared with control at all timepoints. For serial data, the main effects of treatment were tested using two-factor ANOVA for repeated measures, and differences between the treatments at specific time points were compared using the student-Newman-Keuls test. P<0.05 was significant. Systat 13.0 was used for MRI linear modeling, nesting of wall motion agreement between parameters. Kaplan-Meier was used for survival analysis.

### Example 1

### Fluoroscopy Images

Fluoroscopy images were acquired during the cardiac ischemic reperfusion surgery, before, during, and after ischemia. Multiple images were taken with and without contrast and balloon inflation and deflation to confirm restricted blood flow between the first and second diagonal branches of the LAD. The images showed that in swine numbers 6669, 7274, and 7278, that restricted blood flow was achieved. In animal 7270 the images showed that the balloon occlusion occurred at the level of the first diagonal branch, suggesting that more of the left ventricle was impacted by ischemia compared to the other animals. One pig #6668, was complicated by coronary artery dissection during injection and treated with Control virus. Fluoroscopy images confirmed that all four animals (#6669, 7270, 7274, and 7278) had significant cardiac ischemic injury following a sixty-minute balloon occlusion of the LAD.

### Intracardiac Echocardiographs (ICE)

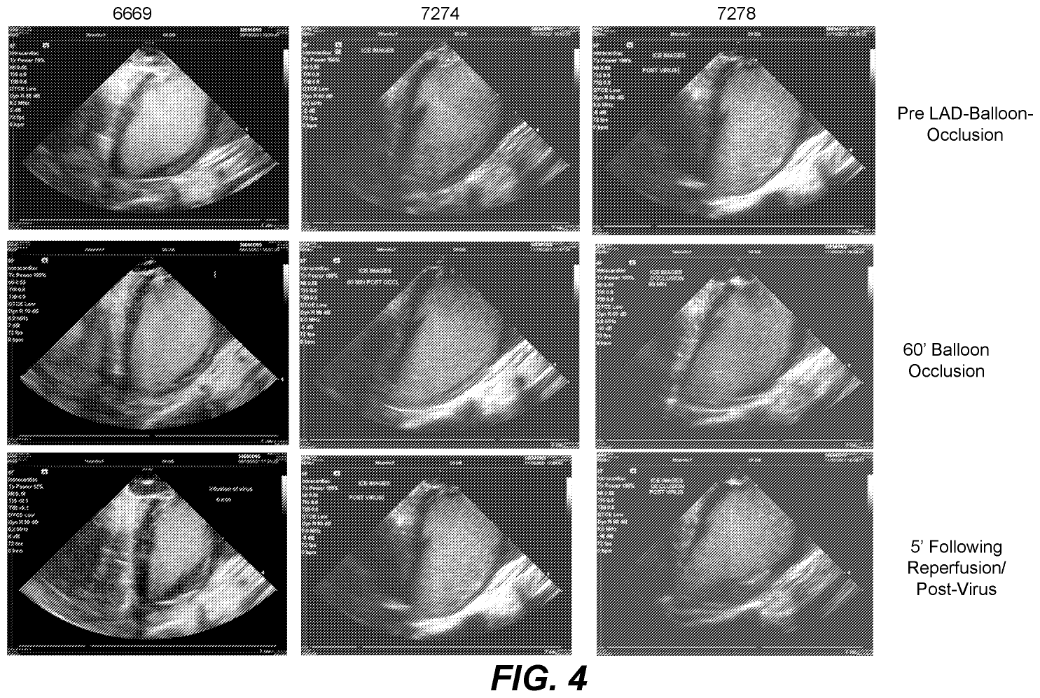

Intracardiac echocardiography images were acquired before ischemia, thirty minutes after ischemia was initiated and at reperfusion during the surgical procedure. The images (FIG. 4) confirmed regional ischemia in the wall of the left ventricle of all animals. Regional ischemia occurred in all animals.

Lactate Dehydrogenase (LDH) and Creatine Kinase (CK) levels

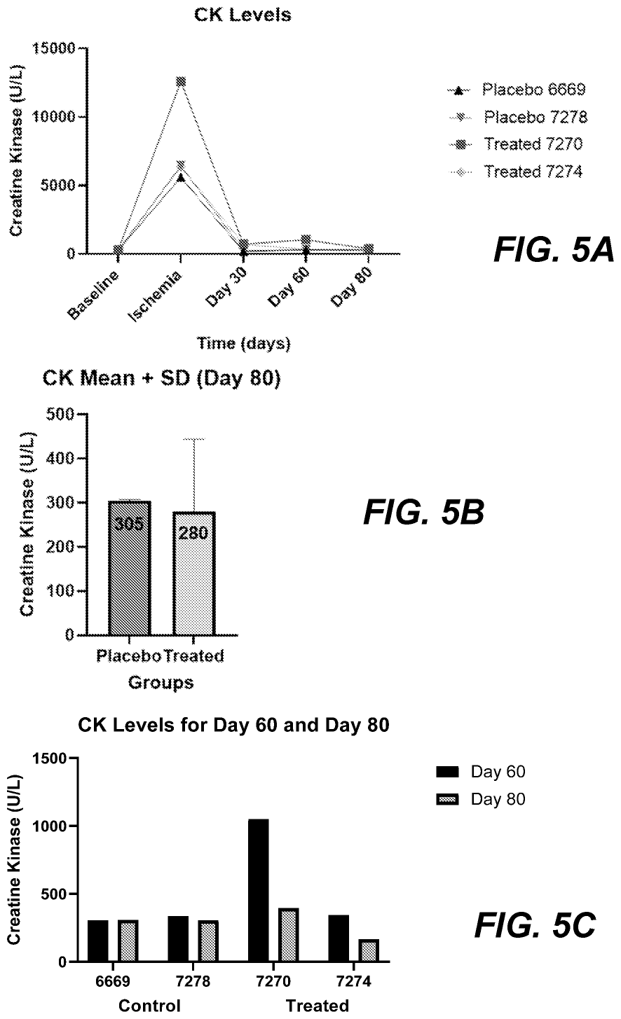

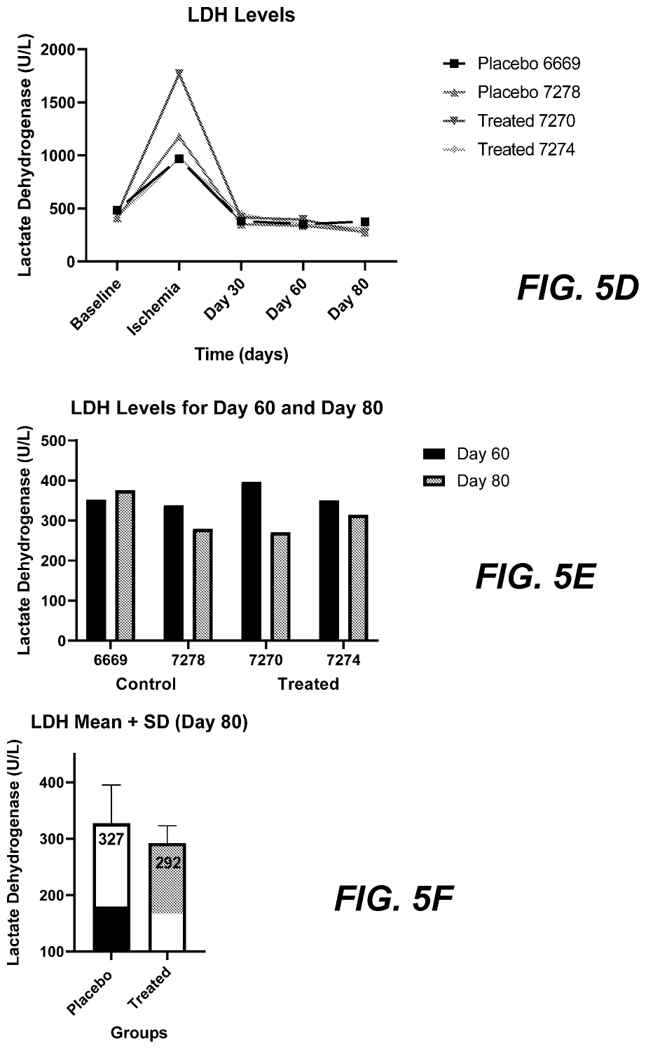

The analysis was conducted on the blood taken before, during, and after ischemia. Sixty minutes following reperfusion, levels of both enzymes were elevated in the blood over the baseline, indicative of cardiac muscle injury (FIG. 5A and FIG. 5D). In all animals, just following reperfusion, creatine kinase (7270>7278>7274>6669) and lactate dehydrogenase (7270>7278>7274>6669) blood levels were significantly higher than the reference range. In pig 7270, levels of both enzymes were higher compared to the other pigs, as a result of balloon occlusion before the first diagonal branch of the LAD, resulting in significantly more muscle damage, and is consistent with the fluoroscopy images.

CK (FIG. 5B) and LDH levels (FIG. 5F) returned to near baseline levels on Day 80 and were not different or slightly lower in treated versus untreated animals when values from two animals were averaged. However, when looking at individual treated animals 7270 and 7274, a repeat dose of IV-administered JBT-miR2-ADD reduced blood levels of CK levels between Day 60 and Day 80 (FIG. 5C), indicating a benefit of the drug versus control virus.

### MRI Images and Analysis

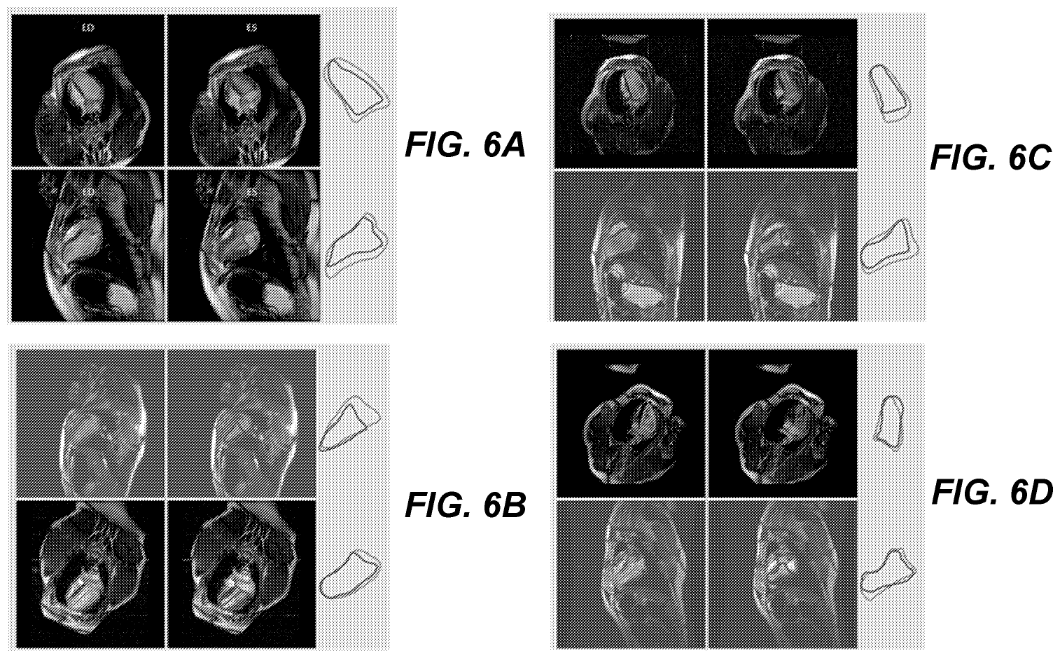

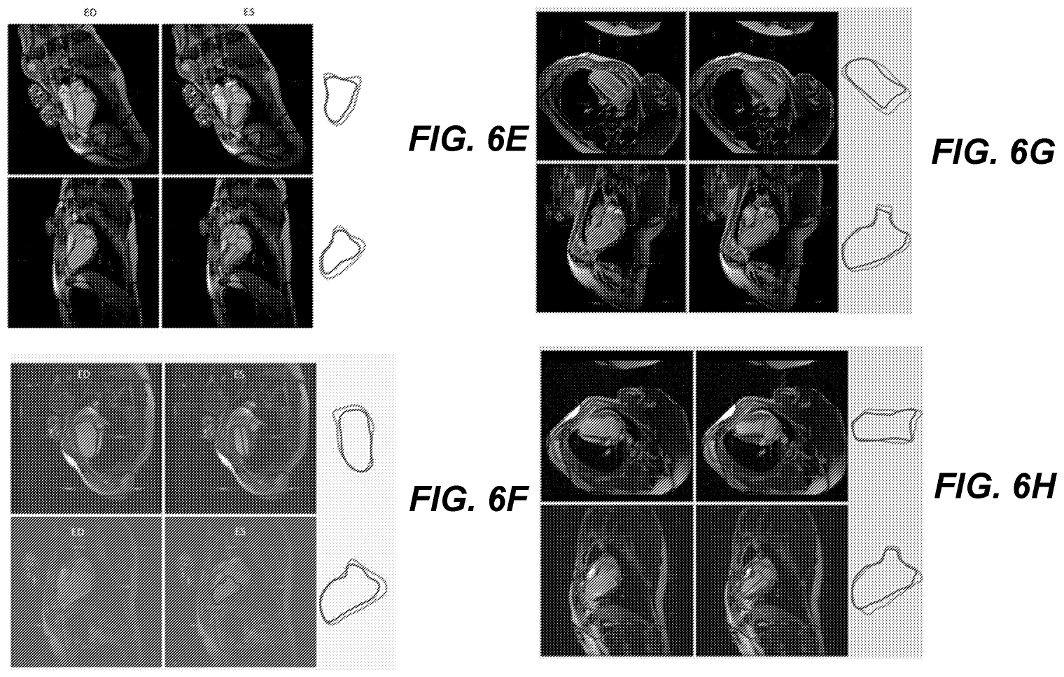

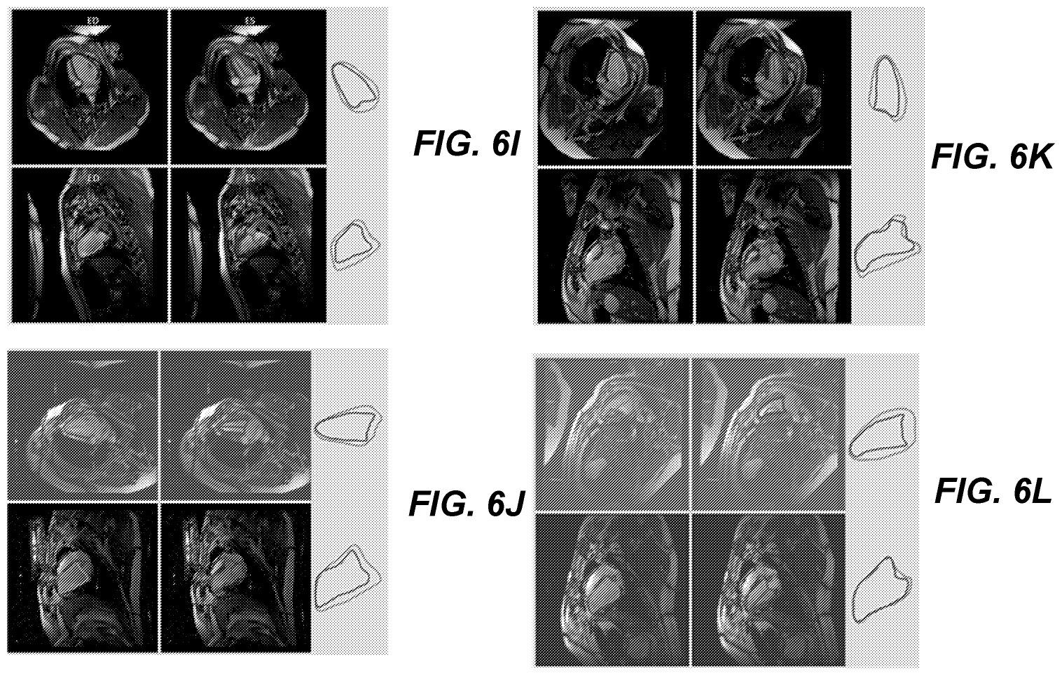

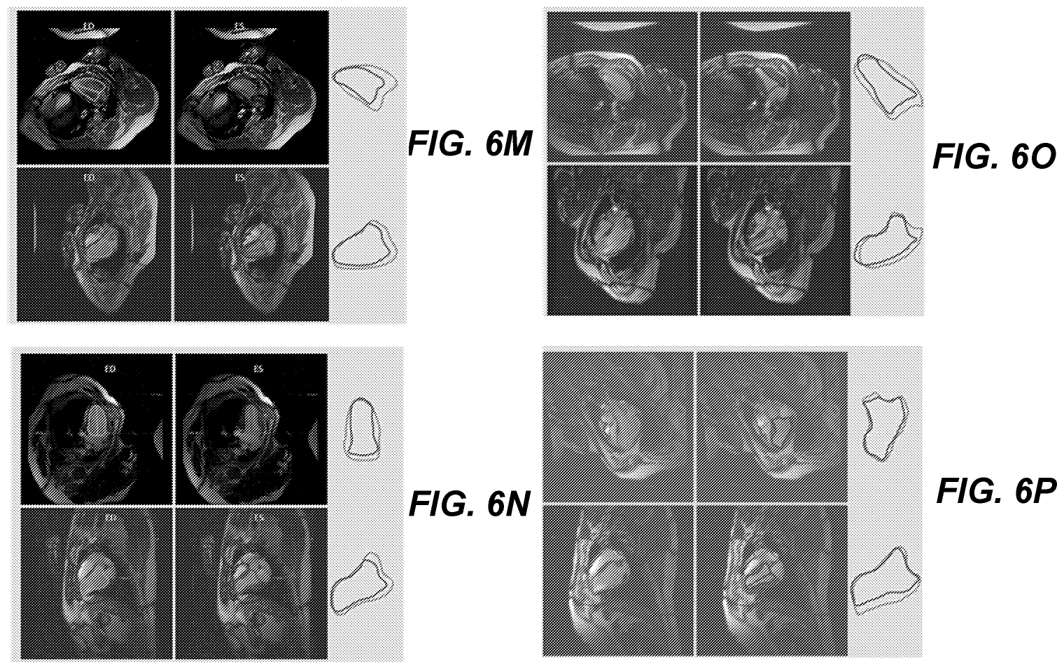

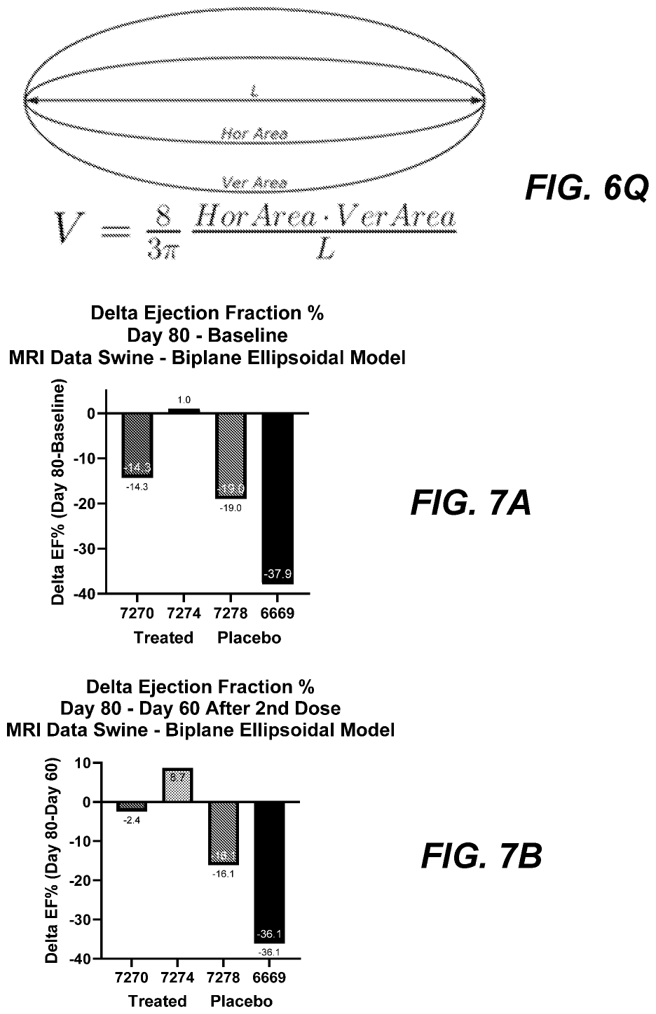

FIG. 6A-FIG. 6P show the biplane ellipsoidal model calculation of heart volumes images. LV volume is calculated as:

$$V = \frac{8}{3 π} \times \frac{HorArea \times VerArea}{L} \qquad ( 2 )$$

MRI Ejection Fraction results are summarized in Table 4 below.

TABLE 4
ABSOLUTE EJECTION FRACTION MRI DATA
SWINE-BIPLANE ELLIPSOIDAL MODEL

| | EF % | | | | | |
|---|---|---|---|---|---|---|
| | | Day | Day | Day | Delta (Day | Delta (Day |
| Swine | Baseline | 30 | 60 | 80 | 80-Baseline) | 80-Day 60) |
| 7270 | 142.6 | 35.2 | 30.7 | 28.3 | −14.3 | −2.4 |
| 7274 | 54 | 49 | 46.3 | 55 | 1 | +8.7 |
| 7278 | 49.7 | 43.5 | 46.8 | 30.7 | −19 | −16.1 |
| 6669 | 54.5 | 41.1 | 52.7 | 16.6* | −37.9 | −36.1 |

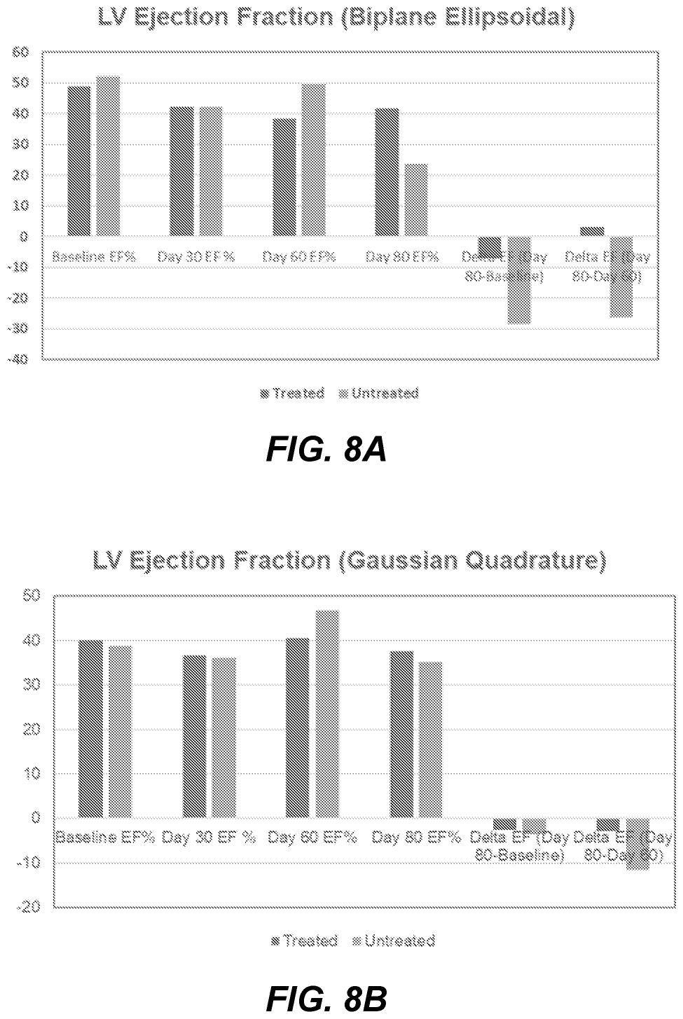

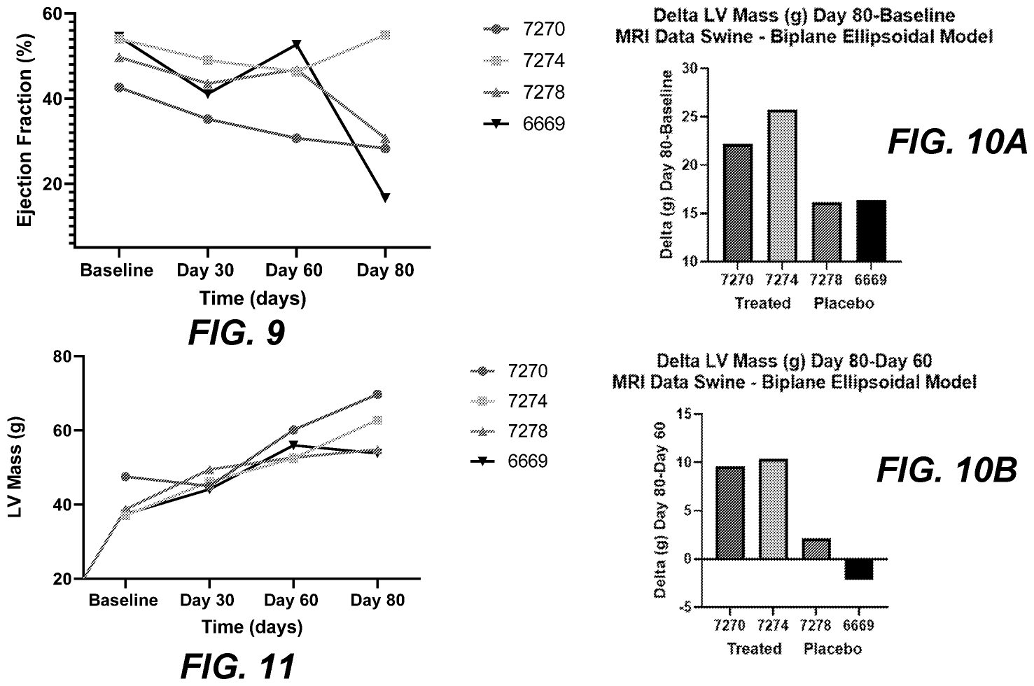

Using the Biplane Ellipsoidal method, serial evaluations of LV volumes and mass were derived from MRI images, using the horizontal and vertical long axis views, and calculated based upon a biplane ellipsoidal model (“EF” Plug-In, OsiriX MD). The delta EFs between Day 80 and Baseline for JBT-miR2-ADD treated animals 7270 and 7274 were −14.3%, and +1% respectively. In comparison, the delta EFs for the control animals 7278 and 6669 were EF −19% and −37.9%, respectively, showing significant depressed function at Day 80. A second dose of drug on Day 60 appeared to be effective in increasing EF because the delta EFs between Day 80 and Day 60 were −2.4% and +8.7% for the JBT-miR2-ADD treated animals 7270 and 7274, compared to −16.1% and −36.1% (Table 4, FIG. 7A-FIG. 7B). The mean average absolute values of EF for treated versus untreated animals were calculated using the Gaussian Quadrature and compared to the Biplane Ellipsoidal method (FIG. 8A-FIG. 8B). Both methods showed similar patterns of EF changes. On Day 80, treated animals had a greater EF compared to untreated animals. The delta EFs between Day 80 and baseline and between Day 80 and Day 60 were also positive in treated animals. The absolute EF for all animals is shown in FIG. 9. Between Day 60 and Day 80, the EF decreased significantly in the untreated pigs 7278 and 6669. On the contrary, in the treated pigs 7270 and 7274, the EF on Day 80 remained similar to that on Day 60 in pig 7270 and increased to normal levels in pig 7274.

Furthermore, MRI LV Mass (g) MRI Data obtained using Swine Biplane Ellipsoidal Model is also summarized below in Table 5.

TABLE 5
LV MASS (G) MRI DATA

| | | | | | Delta (g) | Delta (g) |
|---|---|---|---|---|---|---|
| | Baseline | Day | Day | Day | (Day 80- | (Day 80- |
| Swine | (g) | 30 (g) | 60 (g) | 80 (g) | Baseline) | Day 60) |
| 7270 | 47.54 | 44.97 | 60.12 | 69.71 | 22.17 | 9.59 |
| 7274 | 37.09 | 46.18 | 52.48 | 62.82 | 25.73 | 10.34 |
| 7278 | 38.70 | 49.49 | 52.66 | 54.84 | 16.14 | 2.18 |
| 6669 | 37.44 | 44.14 | 55.98 | 53.84 | 16.4 | −2.14 |

In this example, LV mass increased in JBT-miR2-ADD treated animals compared to control (Table 5, FIG. 10A-FIG. 10B and FIG. 11). The increase in both EF and LV mass was primarily due to the second IV dose of JBT-miR2-ADD, suggesting that multiple monthly doses may be required.

### 3D MRI LV Construction and Wall Motion Analysis

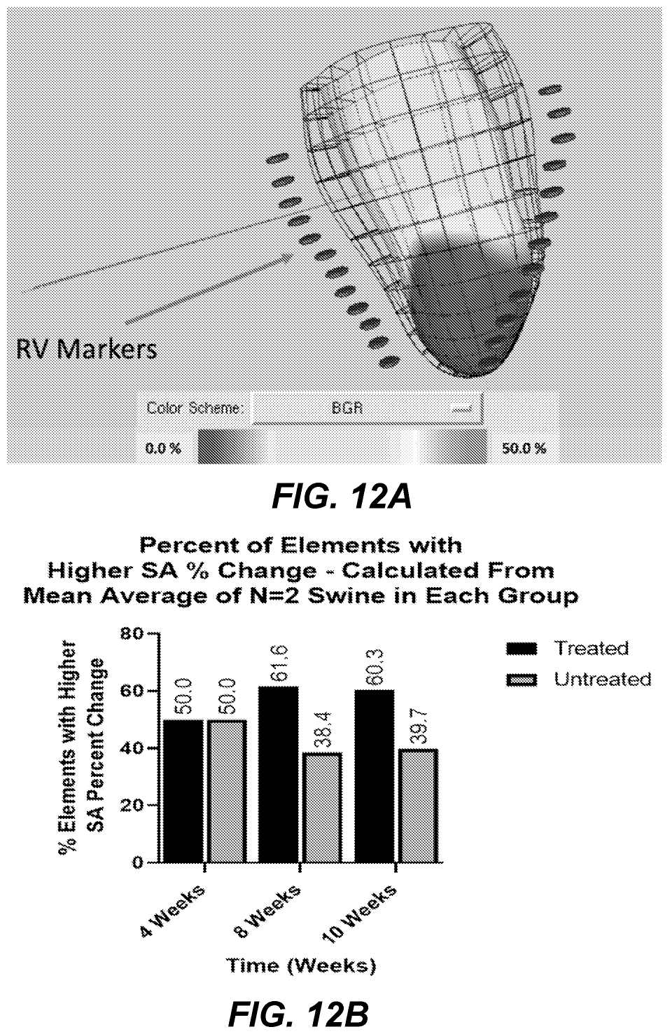

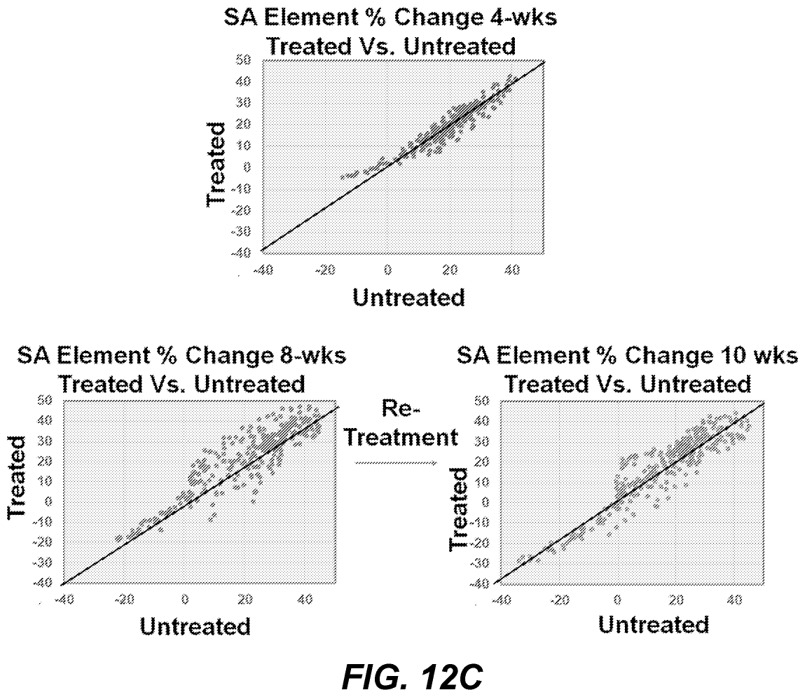

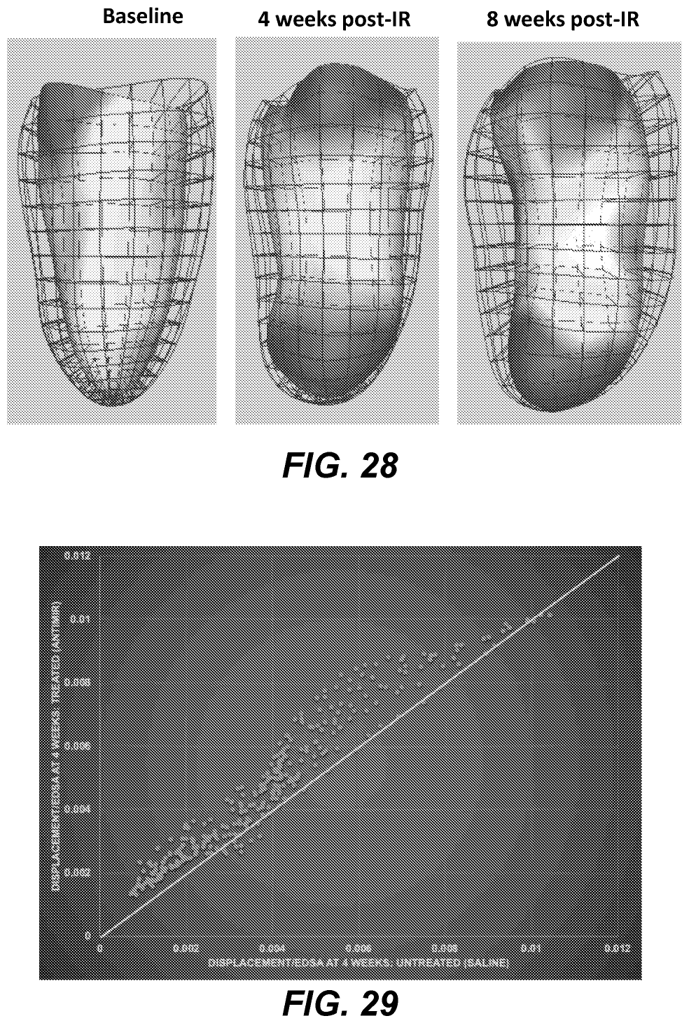

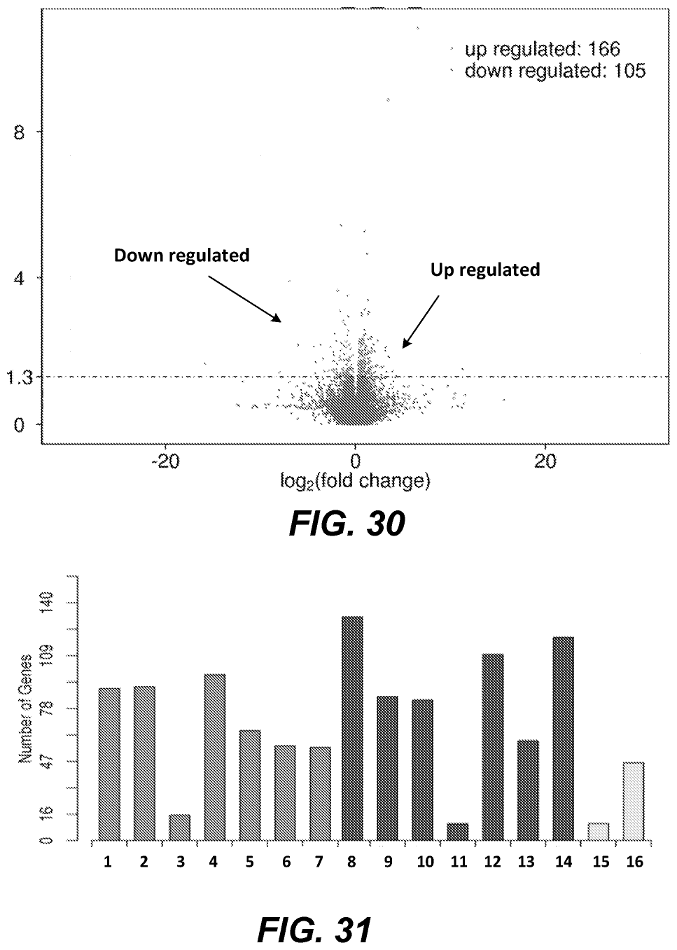

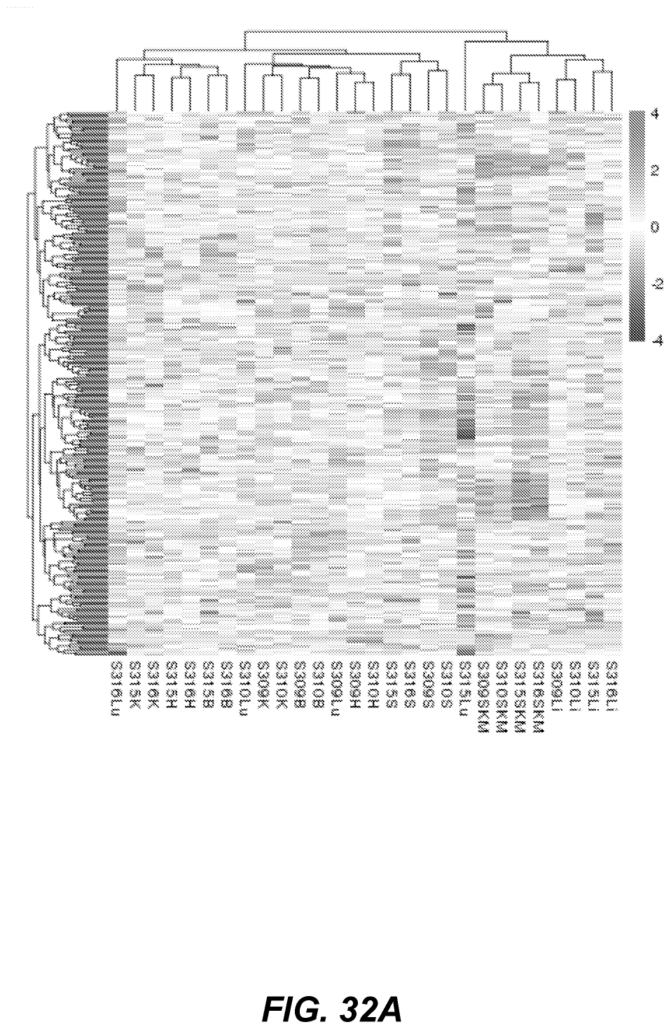

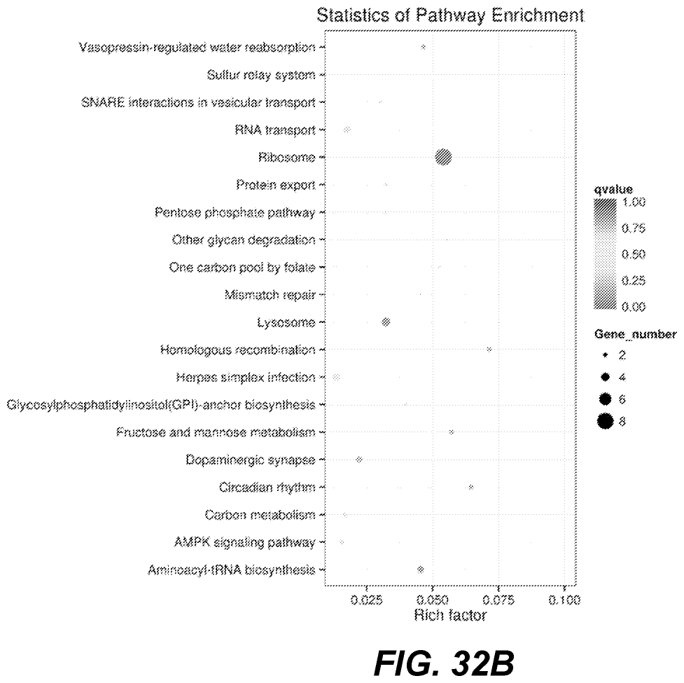

LV regional wall motion was calculated by 3D reconstruction of MR images, using 9-10, sequentially stacked short axis slices (5 mm thick with no gaps), acquired at a spatial resolution of 1.5 mm by 1.5 mm from base to apex and registered perpendicular to the long axis constructed from the middle of the mitral valve annulus to the apex. The 3D contours at ED and ES phases were then fitted, by a least square's routine, to a prolate spheroid, with 300 equidistant nodes on its surface. Multiple four-sided finite surface elements were also created, and the % area change between ED and ES of each element provided a composite measure of circumferential and longitudinal regional strain. To ensure the spatial correspondence in serial evaluations through time, the nodes and elements were positioned in relation to fiducial anatomical loci in the RV (FIG. 12A). Using this method, 3D MRI LV control pig ischemic reperfusion models were constructed for baseline, 4 weeks pst IR and 8 weeks post IR and are shown in FIG. 28. ReconstructionRegional shortening was then parametrically color-coded on the end-systolic volume image. An early comparison (untreated pigs=2; treated pigs=2) of group averages of corresponding elements was performed. Using an x-y display, with line of identify between untreated and treated, to track change over time, a preponderant number of surface area elements shifted above the line of identity (improvement with JBT-miR2-ADD) at 4-, 8- and 10-wks of follow-up (FIG. 12C), suggesting that treatment was associated with a salutary shortening effect on both ischemic and non-ischemic areas of myocardium, similar to observations in mice. At 8-wks and 10-wks, 60% of the nodes in the JBT-miR2-ADD treated animals had an increase in displacement compared to the untreated animals when averaged (FIG. 12B).

### Assessment Cardiac CT Data

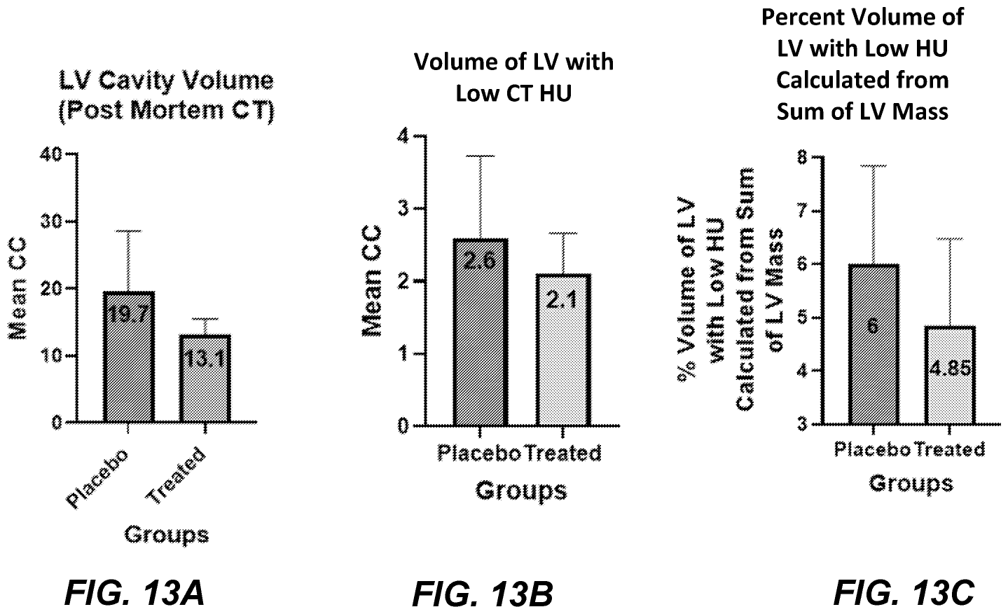

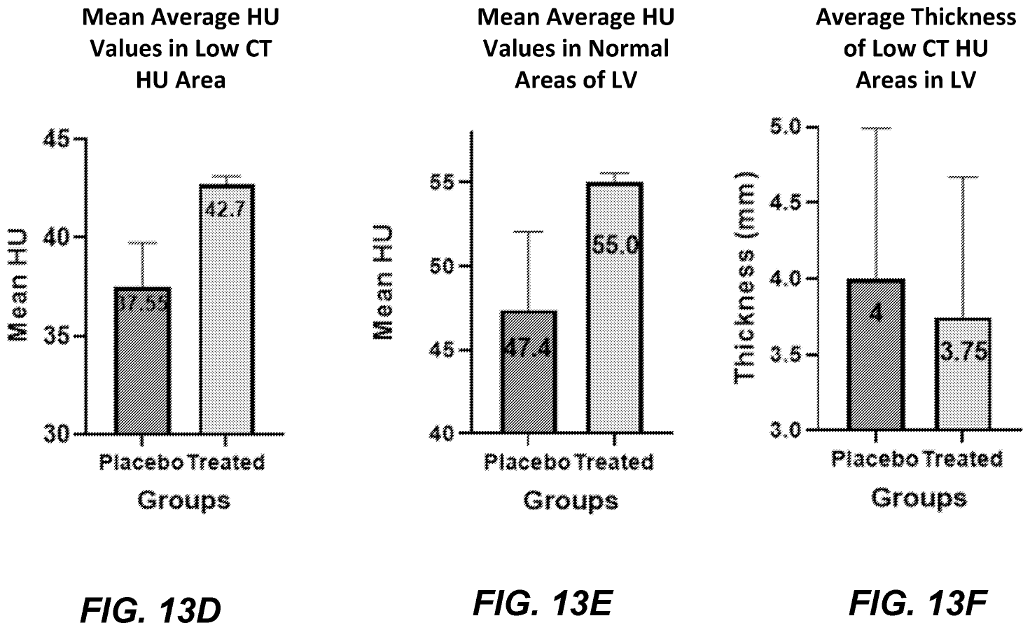

At 10-wks post-IR the heart was excised and submerged in ice for post-mortem CT. Hearts were imaged using a GE CT scanner, 256 row detectors, 70 kVp, 500 mA and an image spatial resolution of 0.625 mm/per slice. Between 10 and 12 slices were analyzed from the base to the apex of the heart. LV mass, LV wall thickness and Hounsfeld units (HU) were measured in the short axial image from the base to apex, and the sum, or average of all measures for each heart were calculated. The low CT HU area was determined by vision, by distinguishing objects with 4-5 CT HU difference. Overall, cavity volumes were smaller (less dilation) in the treated hearts, compared to placebo hearts (33% reduction in cardiac volumes in treated group, Table 6, FIG. 13A). There were less infarcted myocardium and smaller scars (e.g., low HU (lower HUs were consistent with fatty replacement of the myocardium), and less infarcted tissue (FIG. 13B-FIG. 13C). Specifically, FIG. 13B shows 19% reduction in area of LV with low HU (e.g., scar) in treated group, and FIG. 13C shows 19% reduction in volume of scar of LV in treated group. Within the scar, the HUs were higher in JBT-miR2-ADD treated hearts (FIG. 13D-FIG. 13E) (consistent with less fatty replacement, and necrotic tissue). Specifically, FIG. 13D shows that HU were 13.7% higher in low CT HU area in treated animals consistent with less scar; FIG. 13E shows that HU were 16% higher in normal areas of LV in treated animals consistent with less scar; and FIG. 13F shows 6% reduction in thickess of area of scar in treated animals. The average thickness of the scar was smaller in the treated hearts, compared to the control hearts (FIG. 13F). Overall, all positive signs that the scar was smaller and the positive effect on the myocardium were observed in the treated population (FIG. 13A-FIG. 13F).

TABLE 6
MEAN AVERAGE OF TREATED AND UNTREATED CARDIAC CT
PARAMETERS

| | | | | | | | | | % |
|---|---|---|---|---|---|---|---|---|---|
| Case | Control | | Mean | SD | Treated | | Mean | SD | change |
| number | 6669 | 7278 | | | 7270 | 7274 | | | |
| LV mass volume cc | 38.6 | 46.8 | 42.7 | 5.80 | 41.7 | 45.5 | 43.6 | 2.69 | 2.11 |
| LV mass volume = | 40.8 | 49.3 | 45.05 | 6.01 | 43.9 | 47.9 | 45.9 | 2.83 | |
| (volume × 1.053 g/cc) | | | | | | | | | |
| LV cavity volume = cc | 26 | 13.4 | 19.7 | 8.91 | 14.8 | 11.4 | 13.1 | 2.40 | −33.50 |
| Volume with a low CT HU cc | 1.8 | 3.4 | 2.6 | 1.13 | 2.5 | 1.7 | 2.1 | 0.57 | |
| % volume with low HU of sum LV | 4.7 | 7.3 | 6 | 1.84 | 6 | 3.7 | 4.85 | 1.63 | −19.17 |
| mass. | | | | | | | | | |
| HU value of low CTHU area = HU | 39.1 | 36 | 37.55 | 2.19 | 42.4 | 43 | 42.7 | 0.42 | |
| HU value in normal areas | 44.1 | 50.7 | 47.4 | 4.67 | 55.4 | 54.7 | 55.05 | 0.49 | |
| Average thickness of low CTHU | 3.3 | 4.7 | 4 | 1.00 | 3.1 | 4.4 | 3.75 | 0.92 | −6.25 |
| area mm | | | | | | | | | |
| LV anterior, posterior, septal and | 6.5-9.1 | 7.7-10.9 | | | 5.2-12.4 | 5.6-10.9 | | | |
| lateral walls mm | | | | | | | | | |

TABLE 7
WALL THICKNESS DATA

| | (mm) | | | | <35 mm (mm) | | | |
|---|---|---|---|---|---|---|---|---|
| | Anterior | Septal | Posterior | Lateral | Anterior | Septal | Posterior | Lateral |
| 6669 | 8.58 | 6.49 | 8.65 | 9.08 | 6.92 | 4.4 | 7.62 | 8.57 |
| 7278 | 10.92 | 7.7 | 8.42 | 10.44 | 8.68 | 6.66 | 5.94 | 9.48 |
| Mean | 9.75 | 7.10 | 8.54 | 9.76 | 7.80 | 5.53 | 6.78 | 9.02 |
| SD | 1.65 | 0.86 | 0.16 | 0.96 | 1.25 | 1.60 | 1.19 | 0.65 |
| 7270 | 5.19 | 11.81 | 12.42 | 8.74 | 3.23 | 11.82 | 13.12 | 8.25 |
| 7274 | 7.74 | 7 | 9.8 | 10.23 | 5.64 | 5.68 | 10.36 | 10.86 |
| Mean | 6.47 | 9.41 | 11.11 | 9.49 | 4.44 | 8.75 | 11.74 | 9.55 |
| SD | 1.81 | 3.40 | 1.85 | 1.05 | 1.70 | 4.34 | 1.95 | 1.85 |
| T-test | 0.02 | 0.58 | 0.28 | 0.14 | 0.06 | 0.58 | 0.07 | 0.64 |

The wall thickness data showed that the anterior wall was thicker in control animals than in the animals treated with JBT-miR2-ADD. In contrary, the septal and lateral walls were thicker in animals with JBT-miR2-ADD treatment than in control. The data followed the same pattern in <35 mm sections.

TABLE 8
THE LOCATION OF LOW CTHU AREA

| Segments | 7 | 8 | 9 | 13 | 14 | 15 | 17 |
|---|---|---|---|---|---|---|---|
| 6669 | — | 50% | — | 33% | 100% | — | |
| 7270 | 100% | — | — | 100% | — | — | |
| 7278 | — | 33% | — | — | 66% | 66% | |
| 7274 | 33% | 66% | — | 100% | 66% | — | |

The MRI and CT data confirms that 1) two single doses of IC and IV administered JBT-miR2-ADD that delivers inhibitors to miR-99, miR-100, let-7a and let-7c is necessary and sufficient to re-activate an underlying cardiac regeneration process in pig hearts with IR injury increasing LV EF, increasing cardiac mass, and reducing scar in the LV.

### Example 2

### Clinical Chemistry Test

Clinical chemistry data were compared between treated and untreated animals. There were no real differences between the treated and untreated animals at any time point.

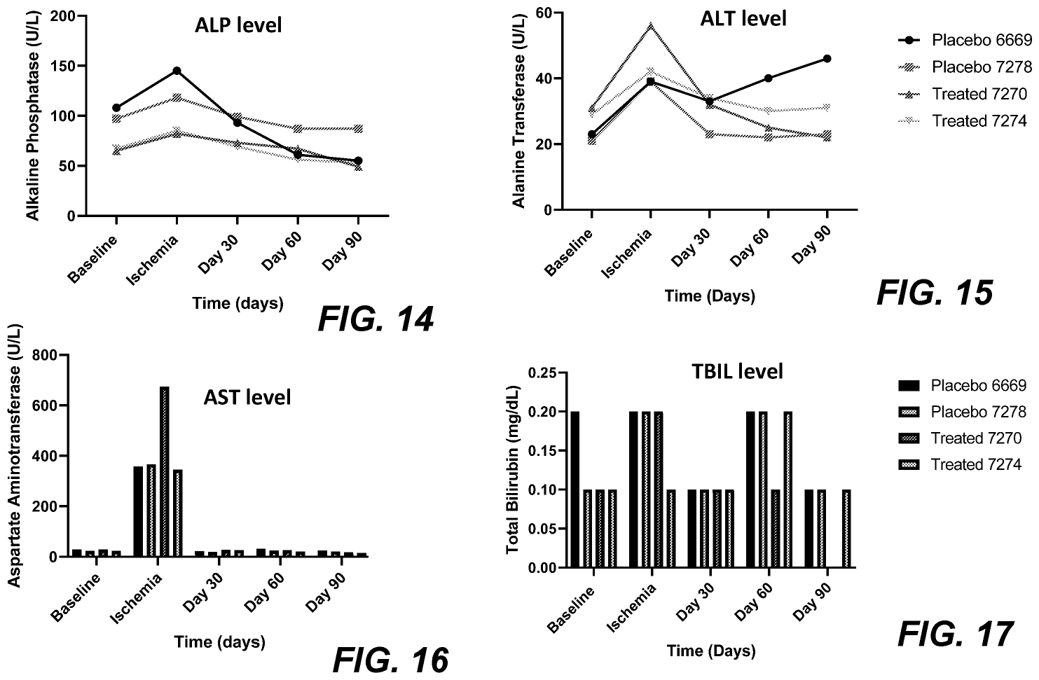

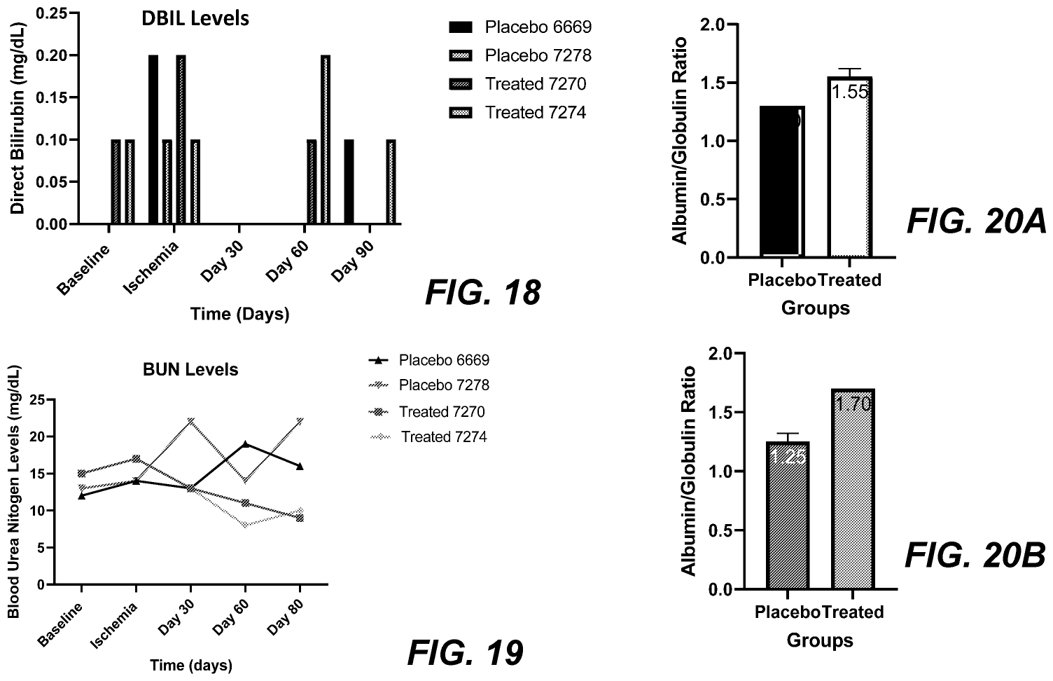

There were no differences between treated and untreated animals for liver enzymes (ALT, ALP, AST, TBIL, DBIL, FIG. 14-FIG. 18). In all these animals, there was a transient increase in the circulating levels of these enzymes during the ischemia, but the levels all returned to normal by Day 30. A repeat dose of JBT-mIR2-ADD on Day 60 did not further increase liver enzyme levels, suggesting no effects of this virus on liver function.

BUN levels were generally lower on Day 60 and Day 80 in the two JBT-mIR2-ADD treated pigs compared to control, suggesting that JBT-miR2 mitigated cardiac muscle injury and aids the kidneys in removing urea from the blood (FIG. 19). Persistent high BUN is associated with increased risk of CV events in patients with acute HF and is a predictor of mortality. Specifically in pig 7278 (control), BUN level (22 mg/dL) was higher than the reference range (9-21 mg/dL), after Day 80. In the 7274 (JBT-mIR2-ADD treated pig), BUN level (8 mg/dL) was lower than the reference range (9-21 mg/dL) on Day 60 but increased to within the reference range by Day 80 (raised to 10 mg/dL).

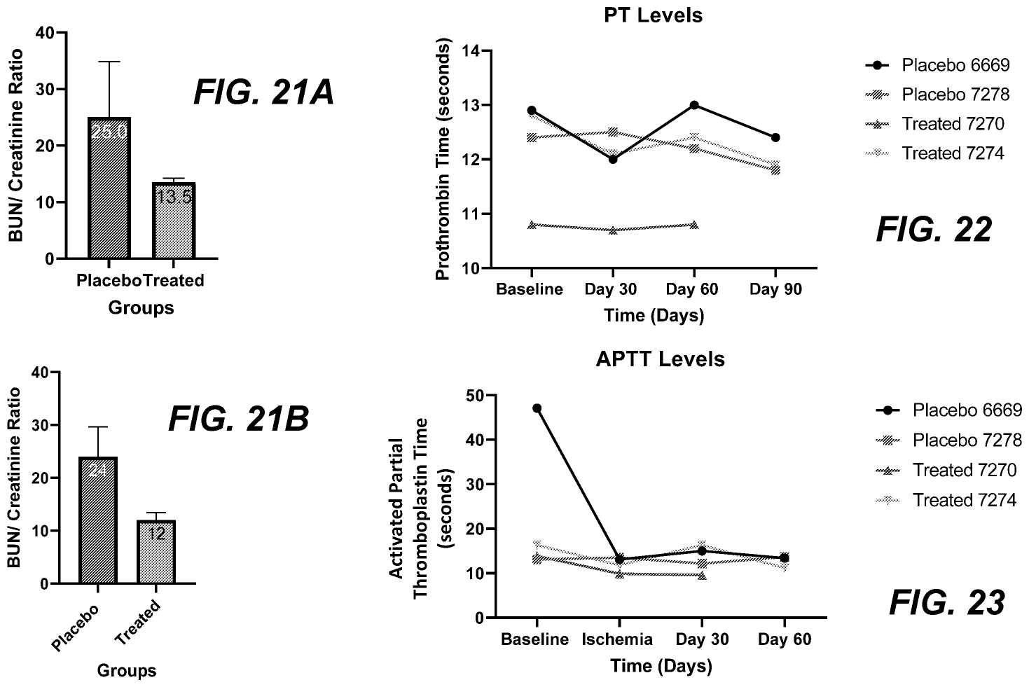

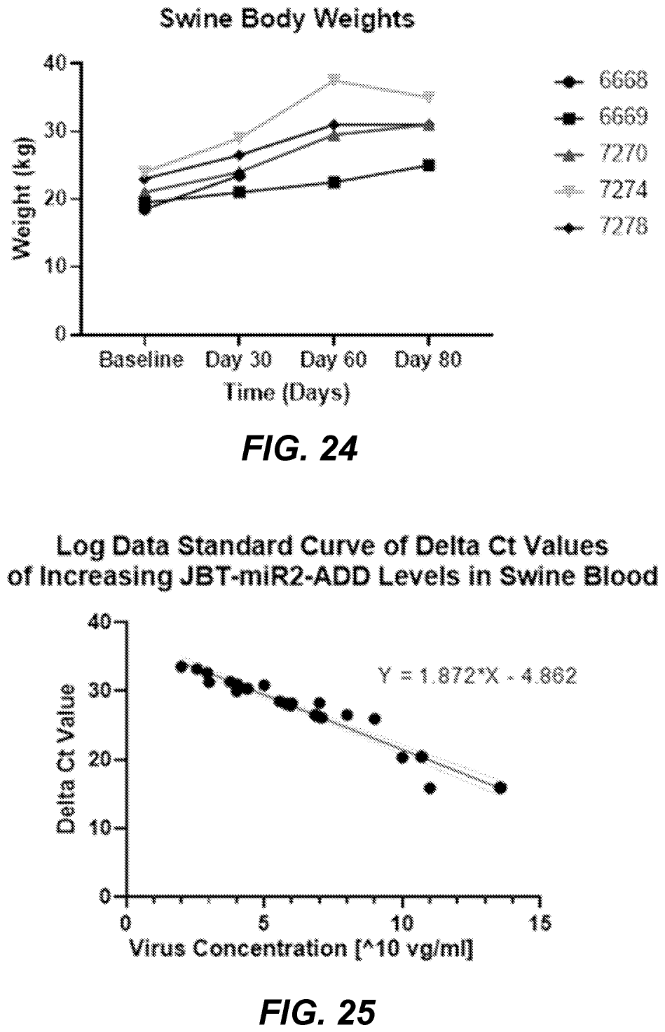

The Albumin/Globulin (A/G) ratios were higher in the treated than in placebo animals on Day 60 and 80 (FIG. 20A and FIG. 20B). The Blood Urea Nitrogen/Creatine ratios were lower in the treated than in placebo treated animals on Day 60 and 80 (FIG. 21A and FIG. 21B). To ensure the quality of data, baseline levels of the enzymes and chemicals all animals were tested. At baseline, ALT levels were higher in the treated animals 7274 and 7270 compared to the untreated animals. Bilirubin was undetectable in the treated animals. All other values were in the normal range and not of any concern. During ischemia, there were significant elevations in creatine kinase and lactate dehydrogenase in all animals consistent with the cardiac injury. By comparing the treated and untreated animals, the CK levels were higher in the treated populations (P=0.0027). The A/G ratio was also higher in the treated animals (P=0.00853). At Day 30, the CK levels were higher in the treated than in the untreated animals (P=0.00271). The A/G ratio was also higher in the treated animals (1.8 in treated, 1.2 in untreated, P=0.009). LDH and CK levels are further discussed in Example 1. There were no differences between treated and untreated animals in any clinical chemistry analyte on Day 60. After the Day 60 MRI, swine 7270 and 7274 were re-dosed with JBT-mRI-ADD by intravenous bolus injection. AST levels were significantly lower in treated animals (P=0.05). All other values were comparable between the two groups.

### Hematology

White blood cells decreased over time post ischemia in control animals. The decrease in white blood cells in JBT-mRI-ADD treated animals was evident in 7274, but not in 7270. Platelet count decreased in animal 6669 on Day 60 and Day 90 with a marginal decrease in 7278. This decrease was also evident in the treated animals 7270 and 7274. Percentage of neutrophils increased in control animals, but not in the treated animals.

Percentage of lymphocytes decreased in control animal 7278. Conversely, in treated animals 7270 and 7274, the percentage of lymphocytes increased. The percentage of reticulocytes increased in animal 7270 and decreased in animal 7278. The values were unchanged in treated animal 7270 but increased in animal 7274.

In all animals, there were no abnormal findings in any of the hematology tests, all results fell within the reference range across all days. In all animals, the platelet count stayed consistently within the reference range (174-745×10³/μL). Red blood cell hematology was similar at all time points in both treated and control animals. In all animals, there were slight levels of anisocytosis (unequally sized RBCs) occurring, indicating that the RBCs in the animals were the same size. There were also slight levels of poikilocytoses (abnormally shaped erythrocytes) and polychromasia (multicolored erythrocytes) in all animals, indicating the increased presence of normally shaped erythrocytes in the blood.

On Day 30, there were no significant differences between baseline hematology values in treated and untreated animals. The percentage red blood cell distribution width (RDW) was 1.1% higher in the treated animals than in the control animals (P=0.02). The number of monocytes (×10³ μl) were 33% lower in the treated animals than in the control animals (P=0.04). On Day 60, the percentage red blood cell distribution width (RDW) was higher in the treated animals than in the control animals (P=0.02). The number of neutrophils (x 103 l) were lower in treated animals than in the control (P=0.01). The number of reticulocytes (×109 L) was 40% higher in treated animals than in the control (P=0.05). On Day 80, the number of lymphocytes (×10³ l) increased by 20% in the treated animals compared to control animals (P=0.02).

### Urinalysis

None of the findings in the urinalysis were of concerning nature, with overall consistency in the findings from Baseline to Day 80 in all animals. Trace amounts of RBCs and bacteria in the urine possibly occurring during the time of catheter insertion. All other urinalysis values appeared to be normal.

### Coagulation

In all animals, PT remained consistent from Baseline to Day 80 (FIG. 22). Swine 6669 (placebo) had an outlier Baseline APTT value, which dropped within the range of the other swine by Day 30. In the other swine, APTT values remained consistent from Baseline to Day 80 (FIG. 23).

### Electrocardiograms

Electrocardiograms (EKG) did not show arrhythmias induced, except for an increase in heart rate (HR) caused by the balloon occlusion, followed by the administration of JBT-mRI-ADD in treated animals and control virus in control animals. Acute ischemic ST-T changes in the tracing were associated with loss of initial forces on the QRS during the ischemia, however these findings essentially return to normal by 30 days.

### Pathology

Hearts, both without fixation at the time of necropsy, and with fixation were examined. Other tissues the spleen, liver, kidney, brain, lung, and skeletal muscle were stored in neutral buffer formalin for fixation prior to paraffin wax embedding, sectioning, and staining. Gross evaluation of tissues taken from each animal after fixation is as following:
(1) 7270: the tissue was difficult to cut, as the myocardium wall was thick; scarring extended continuously from the apex towards the septum, about halfway up the ventricular system; Multiple clots filling almost the whole left atrium and left ventricle, more clotting found than expected.
(2) 7278: scar found from apex to distal ventricular system; a patchy fibrosis was observed in appearance as it sets to the septum, extending a quarter of the way; this heart was very similar to 7274 in appearance, as there was no clot in the left atrium with minimal scarring; appearance of more fat and of a relatively small infarct.
(3) 7274: scarring found was mainly at the apex, extending to the septal wall; at the apex, fibrous membrane was found, with the appearance of scarring from the outside (possibly inflammation); no clotting in the left atrium, as it might have been washed out; clot found sitting on the papillary muscle standing up from the apex.
(4) 6669: scarring found was mainly at the apex, extending to the septal wall; large clot in the left ventricle was removed.

### Vital Signs

6668 experienced a spontaneous coronary artery dissection when the study drug was administered during reperfusion down the LAD, resulting in a tear of the LAD. This resulted in the animal crashing and requiring resuscitation over a 20-minute period. The animal recovered, but again crashed during anesthesia prior to the 4-week cardiac MRI. From this animal, it was noted that any subsequent study drug administration at reperfusion would be administered.

TABLE 9
EKG DATA

| Time | HR (bpm) | | | | CO₂ RR (rpm) | | | | SpO₂ (%) | | | |
|---|---|---|---|---|---|---|---|---|---|---|---|---|
| points | 6669 | 7270 | 7274 | 7278 | 6669 | 7270 | 7274 | 7278 | 6669 | 7270 | 7274 | 7278 |
| 1 | 100 | 148 | n/a | 103 | 8 | 11 | 11 | 10 | 95 | 89 | n/a | 73 |
| 2 | | 90 | | | | 8 | | | | 96 | | |
| 3 | 83 | 129 | 128 | 133 | 8 | 10 | 11 | 10 | 98 | 95 | 98 | 97 |
| 4 | | 138 | 125 | 315 | | 10 | 11 | 10 | | 95 | 98 | 98 |
| 5 | | | 123 | | | | 11 | | | | | |
| 6 | 80 | 208 | 123 | 134 | 8 | 11 | 11 | 10 | 96 | 94 | | 97 |
| 7 | | 111 | 175 | 127 | | 13 | 11 | 10 | | 95 | n/a | 97 |
| 8 | 80 | 224 | 196 | 230 | 8 | 13 | 11 | 10 | 97 | 96 | 95 | 96 |
| 9 | | 114 | 131 | 350 | | 13 | 13 | 10 | | 91 | | 97 |
| 10 | 97 | | | | 10 | | | | 93 | | | |
| 11 | | 110 | | 167 | | 13 | | 10 | | n/a | | |
| 12 | 92 | | 119 | | 10 | | 13 | | 100 | | 97 | |
| 13 | n/a | 94 | 82 | 111 | n/a | 0 | 0 | 0 | n/a | n/a | 94 | 100 |
| 14 | n/a | 121 | 118 | n/a | 0 | 15 | 8 | 6 | n/a | 93 | 97 | n/a |
| 15 | | | | 73 | | | | 6 | | | | 97 |
| 16 | | | | 73 | | | | 7 | | | | 97 |
| 17 | | 133 | | 61 | | 10 | | 8 | | 92 | | 98 |
| 18 | | | | 59 | | | | 8 | | | | 97 |
| 19 | | | | 61 | | | | 8 | | | | 97 |
| 20 | | | n/a | | | | 15 | | | | n/a | |
| 21 | | | 63 | 59 | | | 15 | 8 | | | 90 | 97 |
| 22 | | | | 60 | | | | 8 | | | | 97 |
| 23 | | 96 | | 59 | | 10 | | 8 | | n/a | | 97 |
| 24 | | 94 | 64 | 61 | | 10 | 16 | 8 | | n/a | 93 | 97 |
| 25 | | 93 | 64 | 61 | | 10 | 17 | 8 | | n/a | 94 | 97 |
| 26 | | 88 | 64 | | | 8 | 18 | | | n/a | 95 | |
| 27 | | | 70 | | | | 19 | | | | 95 | |
| 28 | | 87 | | | | 8 | | | | n/a | | |
| 29 | | 88 | 84 | | | 8 | 19 | | | n/a | 97 | |
| 30 | | | 86 | | | | 20 | | | | 97 | |
| 31 | | | | 105 | | | | 10 | | | | 99 |
| 32 | | | | 104 | | | | 10 | | | | 99 |
| 33 | | 104 | 72 | | | 10 | 8 | | | 98 | 97 | |
| 34 | | 105 | | | | 10 | | | | 97 | | |
| 35 | | | 83 | | | | 10 | | | | 97 | |
| 36 | | | 82 | | | | 10 | | | | 96 | |
| 37 | 121 | | | | 17 | | | | 99 | | | |

Heart rate (HR) at baseline were 105 bpm (6668), 100 bpm (6669), 148 bpm (7270) and 103 bpm (7278). For all animals, HR increased during ischemia and reperfusion. By Day 30, 60 and 80 or 100, HRs of all animals returned to baseline levels. Peripheral oxygen saturation (SPO₂) levels were similar between the animals. Not all animals were under anesthesia during the ERG recording and they were intubated and supplemented with oxygen.

### EKG

TABLE 10
MEAN AVERAGE AND SD OF EKG DATA

| (Mean Average ± SD) | | Baseline | Ischemia | Reperfusion | Day 30 | Day 60 | Day 90 |
|---|---|---|---|---|---|---|---|
| Heart Rates | Control | 93.88 ± 12.52 | 125.39 ± 49.06 | 139.67 ± 77.36 | 88.31 ± 40.03 | 138.40 ± 36.02 | 207.00 ± ND |
| (bpm) | | | | | | | |
| | JBT-miR2 | 81.23 ± 17.84 | 116.23 ± 56.89 | 182.25 ± 71.06 | 91.08 ± 6.71 | 83.45 ± 13.22 | 94.63 ± 16.44 |
| Systolic BP | Control | 96.02 ± 3.33 | 87.99 ± 2.65 | 84.91 ± ND | 118.00 ± ND | ND | ND |
| (mmHG) | JBT-miR2 | ND | 93.00 ± ND | ND | ND | ND | ND |
| Arterial | Control | 28.68 ± 53.75 | 47.21 ± 6.80 | 22.44 ± ND | 80.00 ± ND | ND | ND |
| Diastolic BP | | | | | | | |
| (mmHG) | JBT-miR2 | ND | 84.00 ± ND | ND | ND | ND | IND |
| Arterial BP | Control | 56.11 ± 36.92 | 78.82 ± 20.83 | 46.44 ± ND | 92.00 ± ND | ND | ND |
| (mmHG) | JBT-miR2 | ND | 88.00 ± ND | ND | ND | ND | ND |
| Temperature | Control | 96.02 ± 2.84 | 97.94 ± 3.12 | 96.16 ± 4.28 | 98.60 ± ND | ND | ND |
| (° F.) | JBT-miR2 | 88.98 ± ND | 96.58 ± 0.95 | 96.90 ± 0.14 | ND | ND | ND |
| Non-Invasive | Control | 107.78 ± 14.33 | 105.44 ± 18.38 | 103.80 ± 26.30 | 92.50 ± 38.89 | ND | IND |
| Systolic BP | | | | | | | |
| (mmHG) | JBT-miR2 | 120.00 ± ND | 83.75 ± 15.20 | 89.00 ± ND | ND | ND | ND |
| Non-Invasive | Control | 54.61 ± 3.77 | 52.27 ± 14.99 | 51.90 ± 15.56 | 55.00 ± 32.52 | ND | ND |
| Diastolic BP | | | | | | | |
| (mmHG) | JBT-miR2 | 50.50 ± ND | 45.50 ± 2.12 | 35.00 ± ND | ND | ND | ND |
| ET CO₂ | Control | 30.83 ± 4.53 | 31.60 ± 1.73 | 28.73 ± 3.23 | 39.80 ± 3.95 | ND | 27.00 ± ND |
| (mmHG) | JBT-miR2 | 29.08 ± 1.41 | 30.98 ± 4.21 | 30.83 ± 1.17 | ND | 32.13 ± 12.05 | 35.00 ± 4.24 |
| Inhaled CO₂ | Control | 0.01179941 ± 0.02 | 0.354761905 ± 0.55 | 0.119504893 ± 0.18 | 2.90 ± 2.68 | ND | 0 ± ND |
| (mmHG) | JBT-miR2 | 0 ± 0 | 0.3125 ± 0.44 | 0 ± 0 | ND | 1.2 ± 1.69 | |
| CO₂ RR (mg | Control | 759.00 ± 1.41 | 756.07 ± 5.17 | 759.33 ± 1.15 | 758.00 ± 2.82 | 761.50 ± 3.53 | 759.00 ± ND |
| CO₂/kg-h) | JBT-miR2 | 760.00 ± 2.82 | 758.00 ± 0 | 758.00 ± 0 | 761.50 ± 0.70 | 762.00 ± 2.82 | 758.50 ± 0.70 |

Baseline heart rate (HR) was similar between both the JBT-miR2 treated and control groups(Baseline JBT-miR2=81.23+17.84; Baseline Control=93.88+12.52). During ischemia and reperfusion both groups of animals showed an increase in HR. At Day 30, both groups had similar HR. At Day 60 and Day 90, JBT-miR2 treated animals had reduced HR compared to control. At reperfusion, JBT-miR2 treated animals had reduced non-invasive systolic blood pressure (BP) compared to control. At reperfusion, JBT-miR2 treated animals had reduced non-invasive diastolic BP compared to control. End-Tidal (ET) CO₂ and CO₂ reduction reaction (RR) in the control and JBT-miR2 treated animals were similar and consistent throughout baseline to Day 90.

During ischemia and reperfusion both groups of animals showed an increase in HR (Ischemia JBT-miR2=116.23±56.89, Reperfusion JBT-miR2=182.25±71.06; Ischemia Control=125.39±49.06, Reperfusion Control=139.67±77.36). At Day 30, both groups had similar HR (Day 30 JBT-miR2=91.08±6.71; Day 30 Control=88.31±40.03). At Day 60 and Day 90, JBT-miR2 treated animals had reduced HR (Day 60 JBT-miR2=83.45±13.22, Day 90 JBT-miR2=94.63±16.44) compared to Control (Day 60 Control=138.40±36.02, Day 90 Control=207.00±ND). Temperature in the control and JBT-miR2 treated animals were similar and consistent throughout baseline to reperfusion. At reperfusion, JBT-miR2 treated animals had reduced non-invasive systolic BP (Reperfusion JBT-miR2=89.00 ND) compared to control (Reperfusion Control=103.80±26.30). At reperfusion, JBT-miR2 treated animals had reduced non-invasive diastolic BP (Reperfusion JBT-miR2=35.00 ±ND) compared to control (Reperfusion Control=51.90±15.56). ET CO₂ in the control and JBT-miR2 treated animals were similar and consistent throughout baseline to Day 90. CO₂ RR in the control and JBT-miR2 treated animals were similar and consistent throughout baseline to day 90.

### Body Weights

In all animals, there was an overall increase in body weight from Baseline to Day 80. In treated pig 7274, the body weight decreased from 37.5 kg to 35 kg from Day 60 to Day 80. By Day 30 the treated animals 7270 and 7274 had a mean 17.7% increase in body weight from baseline body weight. In untreated animals 6668 and 6669 and 7278, the percentage body weight gain was 14.84% and less than the treated animals (P=0.05). By Day 60, the treated animals mean body weight percent change from baseline was higher than untreated animals (P=0.049) but by Day 80, there was no differences between control and treated animals (P=0.17).

### Pathology

Wet tissues received in formalin were examined grossly, dissected for placement into cassettes and processed into paraffin. Histologic sections were prepared and stained with H&E. Paraffin blocks of tissue remain available for any further analysis, such as immunohistochemical studies, that might be requested. Specimen from 7278, 7270, 7274, and 6669 were examined. Each had brain, spleen, lung, liver, kidney, and muscle available. No specific histologic abnormalities were seen. There were no histologic changes indicative of toxicologic changes amongst the four animals.

### Example 3

Whole blood was collected from the pigs at the following time points indicated in Table 11.

TABLE 11
CT VALUES AND VIRUS LEVEL

| | Time | | | | JBT-miR2-ADD |
|---|---|---|---|---|---|
| Sample | point | | Ct average | Delta Ct | level (vg/mL) |
| 7274 | 0 min, Baseline | | 37.78 | 1.88 | 3970.31337 |
| 7274 | 5 | mins | 21.63 | 18.03 | 1.68253 × 10¹² |
| 7274 | 30 | mins | 22.32 | 17.34 | 7.20065 × 10¹¹ |
| 7274 | 60 | mins | 21.37 | 18.28 | 2.30239 × 10¹² |
| 7274 | 30 | days | 36.98 | 2.68 | 10621.2398 |
| 7274 | 80 | days | 34.63 | 5.03 | 191212.859 |
| 7270 | 0 min, Baseline | | 39.00 | 0.65 | 879.922544 |
| 7270 | 5 | mins | 20.71 | 18.94 | 5.18494 × 10¹² |
| 7270 | 30 | mins | 24.29 | 15.37 | 6.3829E+10 |
| 7270 | 60 | mins | 24.62 | 15.04 | 4.2534E+10 |
| 7270 | 30 | days | 35.96 | 3.69 | 37014.8564 |
| 7270 | 80 | days | 37.07 | 2.59 | 9508.19961 |

The levels of JBT-miR2-ADD were determined in treated animals 7274 and 7270 using a quantitative real-time polymerase-chain reaction assay and standard curve that is specific for the decoy inhibiter 1 in JBT-miR2-ADD. Cycle of quantification (Ct) is a parameter used in real-time PCR, indicating the cycle number where a PCR amplification curve meets a predefined mathematical criterion. Ct averages for all 28 swine samples for treated pigs are shown in Table 11.

Baseline levels in the two pigs were 3970 and 879 vg/mL for treated animals 7274 and 7270, respectively. Five minutes after administration of JBT-miR2-ADD, levels increased between 1.68 and 1.18×10¹² vg/mL. The levels continue to increase, and by Day 30, the levels were 1×10⁴ and 3.7×10⁴ vg/mL. On Day 30, JBT-miR2-ADD levels were 2.67 and 42-fold higher than baseline values for swine 7274 and 7270, respectively.

Repeat dosing of JBT-miR2-ADD on Day 60 increased JBT-miR2-ADD level on Day 80 in pig 7274 (48-fold higher than the baseline value). However, JBT-miR2-ADD level was 10.8-fold higher than the baseline value in retreated pig 7270. This data shows that most of the virus is cleared from the blood 30 days following a bolus administration. However, there was animal to animal variation.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of preventing, inhibiting, reducing, or treating ischemic heart injury, comprising administering a therapeutic composition to a subject before, during, and/or after a cardiac ischemic event,
wherein the therapeutic composition is administrated in a first dose and a second dose separated by a dosing interval.

2. A method of inducing cardiomyocyte regeneration, cardiac repair, vasculogenesis and/or cardiomyocyte differentiation, comprising administering a therapeutic composition to a subject,
wherein the therapeutic composition is administrated in a first dose and a second dose separated by a dosing interval.

3. The method of claim 1 or 2, wherein the therapeutic composition comprises a nucleotide sequence having at least 80% identity, at least 90% identity, or at least 95% identity to SEQ ID NO: 1.

4. The method of any one of claims 1-3, wherein the therapeutic composition comprises a nucleotide sequence of SEQ ID NO: 1, or a nucleotide sequence having no more than ten mismatches to the nucleotide sequence of SEQ ID NO: 1.

5. The method of any one of claims 1-4, wherein the therapeutic composition comprises at least two nucleotide sequences selected from the group consisting of:
(i) a nucleotide sequence having at least 80% identity, at least 90% identity, or at least 95% identity to SEQ ID NOs: 14-18,
(ii) a nucleotide sequence having at least 80% identity, at least 90% identity, or at least 95% identity to SEQ ID NOs: 19-23,
(iii) a nucleotide sequence having at least 80% identity, at least 90% identity, or at least 95% identity to SEQ ID NOs: 24-31, and
(iv) a nucleotide sequence having at least 80% identity, at least 90% identity, or at least 95% identity to SEQ ID NOs: 26-33.

6. The method of any one of claims 1-5, wherein the therapeutic composition comprises a nucleotide sequence of SEQ ID NOs: 17 and 26, or a nucleotide sequence having no more than ten mismatches to the nucleotide sequence of SEQ ID NOs: 17 and 26.

7. The method of any one of claims 1-6, wherein the first dose and/or the second dose are administrated through routes including intracardiac, intramuscular, intravaginal, intravenous, intra-myocardial, intra-ventricular, subcutaneous, systemic, intra-coronary, intraperitoneal, subcutaneous, epicutaneous, intradermal, rectal, intraocular, pulmonary, intracranial, intraosseous, oral, buccal, or nasal.

8. The method of any one of claims 1-7, wherein the first dose and the second dose are administrated through the same or different routes.

9. The method of any one of claims 1-8, wherein the first dose is administrated through intracardiac (IC) infusion.

10. The method of any one of claims 1-9, wherein the first dose is administrated at a rate of 0.1/mL/min-10/mL/min, optionally at a rate of 1/mL/min.

11. The method of any one of claims 1-10, wherein the second dose is administrated intravenously (IV).

12. The method of any one of claims 1-11, wherein the first dose is administrated after the cardiac ischemic event.

13. The method of any one of claims 1-12, wherein the first dose is administrated no more than 30 days after the cardiac ischemic event.

14. The method of any one of claims 1-13, wherein the first dose is administrated before the cardiac ischemic event.

15. The method of any one of claims 1-14, wherein the first dose is administrated during the cardiac ischemic event.

16. The method of any one of claims 1-15, wherein the second dose is administrated at least 1 day after the first dose.

17. The method of any one of claims 1-16, wherein the second dose is administrated at least 30 days after the first dose.

18. The method of any one of claims 1-17, wherein the second dose is administrated before and/or after the first dose is cleared from the subject.

19. The method of any one of claims 1-18, wherein the second dose is administrated when at least 50% of the first dose is cleared from the subject.

20. The method of any one of claims 1-19, wherein the second dose is administrated at a timing that it does not instigate immune response.

21. The method of any one of claims 1-20, wherein the first dose is administered at a dose of at least 1×10¹⁰ vg/kg, optionally at a dose of 2.5×10¹¹ vg/kg.

22. The method of any one of claims 1-21, wherein the second dose is at least 50% of the first dose.

23. The method of any one of claims 1-22, wherein the second dose is administered at a dose of at least 1×10¹⁰ vg/kg.

24. The method of any one of claims 1-23, wherein the second dose is about 1.7×10¹² vg/kg.

25. The method of any one of claims 1-24, wherein the second dose is administrated at the same total amount per subject as the first dose.

26. The method of any one of claims 3-25, wherein the therapeutic composition comprises a viral and/or non-viral vector comprising the nucleotide sequence.

27. The method of any one of claims 3-26, wherein the therapeutic composition comprises a viral vector, optionally the viral vector is selected from the group consisting of adenovirus vectors, adeno-associated virus (AAV) vectors, retrovirus vectors, lentiviral vectors, herpes virus vectors, phages, and poxvirus vectors.

28. The method of claim 27, wherein the viral vector is AAV; optionally the viral vector is selected from the group consisting of AAV 1, AAV 2, AAV 3, AAV 4, AAV 5, AAV 6, AAV 7, AAV 8, AAV 9, and AAV 10 and chimeric AAV derived thereof, and further optionally the viral vector is AAV2/9

29. The method of any one of claims 1-28, wherein the therapeutic composition reduces creatine kinase (CK) and lactate dehydrogenase (LDH) levels, left ventricle (LV) cavity volume, size of scar, blood urea nitrogen (BUN) level, blood urea nitrogen/creatine ratios and anterior wall thickness; and increases LV ejection fraction and LV mass, LV wall displacement/motion, albumin/globulin (A/G) ratios, percentage of lymphocytes, septal and lateral walls thickness.

30. The method of claim 29, wherein
(i) the creatine kinase (CK) and lactate dehydrogenase (LDH) levels, left ventricle (LV) cavity volume, size of scar, blood urea nitrogen (BUN) level, blood urea nitrogen/creatine ratios and/or anterior wall thickness are reduced by at least 5%, at least 10%, at least 20%, at least 30%, or at least 50% compared to the creatine kinase (CK) and lactate dehydrogenase (LDH) levels, left ventricle (LV) cavity volume, size of scar, blood urea nitrogen (BUN) level, blood urea nitrogen/creatine ratios and/or anterior wall thickness of the subject prior to administration with the therapeutic composition; and/or
(ii) the LV ejection fraction and LV mass, LV wall displacement/motion, albumin/globulin (A/G) ratios, percentage of lymphocytes, septal and/or lateral walls thickness are increased by at least 5%, at least 10%, at least 20%, at least 30%, or at least 50% compared to the LV ejection fraction and LV mass, LV wall displacement/motion, albumin/globulin (A/G) ratios, percentage of lymphocytes, septal and/or lateral walls thickness of the subject prior to administration with the therapeutic composition.

31. The method of any one of claims 1-30, wherein the second dose of the therapeutic composition reduces creatine kinase (CK) and lactate dehydrogenase (LDH) levels, left ventricle (LV) cavity volume, size of scar, blood urea nitrogen (BUN) level, blood urea nitrogen/creatine ratios and anterior wall thickness; and increases LV ejection fraction and LV mass, LV wall displacement/motion, albumin/globulin (A/G) ratios, percentage of lymphocytes, septal and lateral walls thickness.

32. The method of claim 31, wherein
(i) the creatine kinase (CK) and lactate dehydrogenase (LDH) levels, left ventricle (LV) cavity volume, size of scar, blood urea nitrogen (BUN) level, blood urea nitrogen/creatine ratios and/or anterior wall thickness are reduced by at least 5%, at least 10%, at least 20%, at least 30%, or at least 50% compared to the creatine kinase (CK) and lactate dehydrogenase (LDH) levels, left ventricle (LV) cavity volume, size of scar, blood urea nitrogen (BUN) level, blood urea nitrogen/creatine ratios and/or anterior wall thickness of the subject prior to administration of the second dose of the therapeutic composition; and/or
(ii) the LV ejection fraction and LV mass, LV wall displacement/motion, albumin/globulin (A/G) ratios, percentage of lymphocytes, septal and/or lateral walls thickness are increased by at least 5%, at least 10%, at least 20%, at least 30%, or at least 50% compared to the LV ejection fraction and LV mass, LV wall displacement/motion, albumin/globulin (A/G) ratios, percentage of lymphocytes, septal and/or lateral walls thickness of the subject prior to administration of the second dose of the therapeutic composition.

33. The method of any one of claims 1-32, wherein the therapeutic composition does not cause arrhythmias, a reduction in a body weight of the subject, liver toxicity, detection of red blood cells in urinalysis, and deviation of hematology values, prothrombin time (PT), activated partial thromboplastin time (APTT), peripheral oxygen saturation (SpO₂), body temperature end-tidal (ET) CO₂ and CO₂ reduction reaction (RR) from clinical reference ranges.

34. The method of claim 33, wherein a lack of liver toxicity is measured by the level of liver enzymes selected from the group consisting of alanine transferase levels (ALT), alkaline phosphatase levels (ALP), aspartate aminotransferase levels (AST), total bilirubin levels (TBIL), and direct bilirubin levels (DBIL).

35. The method of claim 33, wherein the hematology values include percentages of reticulocytes, monocytes, eosinophils, and basophils; counts of platelet, red blood cell, lymphocytes, monocytes, eosinophils, and basophils; and size and shape of erythrocytes and red blood cells.

36. The method of any one of claims 1 and 3-35 wherein ischemic heart injury is myocardial infarction, coronary artery bypass grafting (CABG), cardiac bypass surgery, cardiac transplantation, angioplasty, vascular interventional procedure employing a stent, laser catheter, atherectomy catheter, angioscopy device, beta or gamma radiation catheter, rotational atherectomy device, coated stent, radioactive balloon, heatable wire, heatable balloon, biodegradable stent strut, a biodegradable sleeve, or any combination thereof.

37. The method of any one of claims 1 and 3-36, wherein ischemic heart injury is cardiac ischemic reperfusion injury, optionally wherein the cardiac ischemic reperfusion injury comprises cardiac ischemic damage, cardiac reperfusion injury, or a combination thereof.

38. The method of any one of claims 3-37, wherein the nucleotide sequence comprises one or more chemical modifications.

39. The method of any one of claims 3-38, wherein the nucleotide sequence comprises one or more chemical modifications selected from the group consisting of a modified internucleoside linkage, a modified nucleotide, and a modified sugar moiety, and combinations thereof.

40. The method of claim 39, wherein the one or more chemical modifications comprise a modified internucleoside linkage.

41. The method of claim 41, wherein the modified internucleoside linkage is selected from the group consisting of a phosphorothioate, 2′-Omethoxyethyl (MOE), 2′-fluoro, alkylphosphonate, phosphorodithioate, alkylphosphonothioate, phosphoramidate, carbamate, carbonate, phosphate triester, acetamidate, carboxymethyl ester, and combinations thereof.

42. The method of any one of claims 40 to 41, wherein the modified internucleoside linkage comprises a phosphorothioate internucleoside linkage.

43. The method of any one of claims 39 to 42, wherein at least one of the one or more chemical modifications comprises a modified nucleotide, optionally the modified nucleotide comprises a locked nucleic acid (LNA), and further optionally the locked nucleic acid (LNA) is incorporated at one or both ends of the nucleotide sequence.

44. The method of claim 43, wherein the modified nucleotide comprises a locked nucleic acid (LNA) chemistry modification, a peptide nucleic acid (PNA), an arabino-nucleic acid (FANA), an analogue, a derivative, or a combination thereof.

45. The method of any one of claims 39 to 44, wherein at least one of the one or more chemical modifications comprises a modified sugar moiety.

46. The method of claim 45, wherein the modified sugar moiety is a 2′-O-methoxy ethyl modified sugar moiety, a 2′-methoxy modified sugar moiety, a 2′-O-alkyl modified sugar moiety, a bicyclic sugar moiety, or a combination thereof.

47. The method of any one of claims 45 to 46, wherein the modified sugar moiety comprises a 2′-O-methyl sugar moiety.

48. The method of any one of claims 1-47, further comprising administrating an effective amount of at least one additional therapeutic agent or at least one additional therapy to the subject for a combination therapy.

49. The method of claim 48, wherein each of the therapeutic composition and the at least one additional therapeutic agent or therapy is administered in a separate formulation or are administered together in a single formulation.

50. The method of any one of claims 48-49, wherein the therapeutic composition and the at least one additional therapeutic agent or therapy are administered sequentially, are administered concomitantly, and/or are administered in rotation.

51. The method of any one of claims 48-50, wherein the at least one additional therapeutic agent or therapeutic therapy is selected from the group consisting of Idebenone, Eplerenone, VECTTOR, AVI-4658, Ataluren/PTC 124/Translarna, BMN044/PR0044, CAT-1004, microDystrophin AAV gene therapy (SGT-001), Galectin-1 therapy (SB-002), LTBB4 (SB-001), rAAV2.5-CMV-minidystrophin, glutamine, NFKB inhibitors, sarcoglycan, delta (35 kDa dystrophin-associated glycoprotein), insulin like growth factor-1 (IGF-1) expression, genome editing through the CRISPR/Cas9 system, any gene delivery therapy aimed at reintroducing a functional recombinant version of the dystrophin gene, Exon skipping therapeutics, read-through strategies for nonsense mutations, cell-based therapies, utrophin upregulation, myostatin inhibition, anti-inflammatories/anti-oxidants, mechanical support devices, a biologic drug, a gene therapy or therapeutic gene modulation agent, any standard therapy for muscular dystrophy, and combinations thereof.

52. The method of any one of claims 48-51, wherein the at least one additional therapeutic agent or therapeutic therapy is selected from the group comprising a percutaneous coronary intervention, coronary artery bypass grafting, thrombolytic therapy, anti-platelet therapy, heparin, warfarin, fibrinolytics, oxygen therapy, a vasodilator, pain medication, a beta blocker, an angiotensin converting enzyme (ACE) inhibitor, an angiotensin receptor blocker (ARB), a glycoprotein antagonist, a statin, an aldosterone antagonist, an implantable cardiac defibrillator (ICD), or any combination thereof.

53. The method of any one of claims 1-52, wherein the subject is a mammal, optionally a human.

54. A method of preventing, inhibiting, reducing, or treating ischemic heart injury, and/or inducing cardiomyocyte regeneration, cardiac repair, vasculogenesis and/or cardiomyocyte differentiation comprising administering a therapeutic composition to a subject,
wherein the therapeutic composition comprises a nucleotide sequence having at least 80% identity, at least 90% identity, or at least 95% identity to SEQ ID NO: 1.

55. The method of claim 54, wherein the therapeutic composition comprises a nucleotide sequence of SEQ ID NO: 1, or a nucleotide sequence having no more than ten mismatches to the nucleotide sequence of SEQ ID NO: 1.

56. The method of any one of claims 54-55, wherein the therapeutic composition is administrated to the subject only in a first dose and in a second dose separated from each other by a dosing interval.

57. The method of any one of claims 54-56, wherein the dosing interval is at least 30 days.

58. The method of any one of claims 54-57, comprising reducing lactate dehydrogenase (LDH) levels, left ventricle (LV) cavity volume compared to a subject not administrated with the therapeutic composition.

59. The method of claim 58, wherein the LDH levels and/or LV cavity volume is reduced by at least 20%, at least 30%, or at least 50% compared to the LDH levels and/or LV cavity volume of the subject prior to administration with the therapeutic composition.

60. The method of any one of claims 54-59, wherein the second dose of the therapeutic composition reduces creatine kinase (CK) and lactate dehydrogenase (LDH) levels, left ventricle (LV) cavity volume, size of scar, blood urea nitrogen (BUN) level, blood urea nitrogen/creatine ratios, and/or anterior wall thickness.

61. The method of claim 60, wherein the creatine kinase (CK) and lactate dehydrogenase (LDH) levels, left ventricle (LV) cavity volume, size of scar, blood urea nitrogen (BUN) level, blood urea nitrogen/creatine ratios and/or anterior wall thickness are reduced by at least 20%, at least 30%, or at least 50% compared to the creatine kinase (CK) and lactate dehydrogenase (LDH) levels, left ventricle (LV) cavity volume, size of scar, blood urea nitrogen (BUN) level, blood urea nitrogen/creatine ratios and/or anterior wall thickness of the subject prior to administration of the second dose of the therapeutic composition.

62. The method of any one of claims 54-61, wherein the second dose of the therapeutic composition increases LV ejection fraction and LV mass, LV wall displacement/motion, albumin/globulin (A/G) ratios, percentage of lymphocytes, and/or septal and lateral walls thickness of the subject prior to administration of the second dose of the therapeutic composition.

63. The method of claim 62, wherein the LV ejection fraction and LV mass, LV wall displacement/motion, albumin/globulin (A/G) ratios, septal and/or lateral walls thickness are increased by at least 20%, at least 30%, or at least 50% compared to the LV ejection fraction and LV mass, LV wall displacement/motion, albumin/globulin (A/G) ratios, septal and/or lateral walls thickness of the subject prior to administration of the second dose of the therapeutic composition.


SEQUENCE LISTING (33 sequences)

SEQ ID NO: 1 - DNA, 3593 bp, synthetic construct
  source 1..3593: mol_type = other DNA
  misc_feature 1..3593: note = ADD Stuffer Design 2
  misc_feature 154..180: note = ITR
  misc_feature 340..580: note = U6 Promoter
  misc_feature 751..870: note = miR-99a antagonists
  misc_feature 889..1008: note = let-7a/c antagonists
  misc_feature 1017..1231: note = H1 Promoter
  misc_feature 3453..3593: note = ITR
ccactccctctatgcgcgctcgctcactcactcggccctggagaccaaaggtctccagactgccggcctctggccggcagggccgagtgagtgagcgagcgcgcatagagggagtgggtacctccatcatctaggtttgcccctgcaggcagctgcgcgctcgctcgctcactgaggccgcccgggcgtcgggcgacctttggtcgcccggcctcagtgagcgagcgcgcagagagggagtggccaactccatcactaggggttcctgcggccgcacgcgtctagttattaatagtaatcgaattcgtgttactcataactagtaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgaacaccgacggcgctaggatcatcttgtacccgtagacatttccgatcttgtggttgtattctgtgaccagaatacttgtacccgtagacatttccgatcttgtggttgatgatcctagcgccgtcttttttgagctcaaaaaagacggcgctaggatcatcaacaactatacaaccaatgtactacctcacaagtattctggtcacagaatacaacaactatacaaccaatgtactacctcacaagatgatcctagcgccgtcggatccgagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcactagttatgagtaacacgaattcgattactattaataactagacgcgtgcggccgcaggaacccctagtgatggagttggccactccctctctgcgcgctcgctcactgaggccgggcgaccaaaggtcgcccgacgcccgggcggcctcagtgagcgagcgagcgcgcagctgcctgcaggacatgtgagcaaaaggccagccgtttagtgaaccgtcagatcgcctggagacgccatccacgctgttttgacctccatagaagacaccgggaccgatccagcctccggactctagagagacgtacaaaaaagagcaagaagctaaaaaagatttaaaaattatttttagcgcagttaatggaacaggaactaaatttaccccaaaaatattacgtgaatcaggatataacgttattgaggttgaagagcatgcatttgaagatgaaacatttaaaaatgttgtaaatccaaatccagaatttgatcctgcatgaaaaataccgcttgaatatggtattaaacatgatgcagatattattattatgaatgacccagatgctgacagatttggaatggcaataaaacatgatggtcattttgtaagattagatggaaatcaaacaggaccaattttaattgattgaaaattatcaaatctaaaacgcttaaatagcattccaaaaaatccggctctatattcaagttttgtaacaagtgatttgggtgatagaatcgctcatgaaaaatatggagttaatattgtaaaaactttaactggatttaaatgaatgggtagagaaattgctaaagaagaagataacggattaaattttgtttttgcttatgaagaaagttatggatatgtaattgatgactcagctagagataaagatggaatacaagcttctatattaatagcagaggctgcttgattttataaaaaacaaaataaaacattagtagactatttagaagatttatttaaagaaatgggtgcatattacactttcactttaaacttgaattttaaaccagaagaaaagaaattaaaaattgaaccattaatgaaatcattgagagcaacacccttaactcaaattgctggacttaaagttgttaatgttgaagactacatcgatggaatgtataatatgccaggacaagacttactaaaattttatttagaagataagtcatgatttgctgttcgcccaagtggaactgaacctaaactaaaaatttattttataggtgttggtgaatctgttcaaaacgctaaagttaaagtagacgaaattattaaagaattaaaattaaaaatgaatatataggagaaaaaatgaaactaaacaaatatatagatcacacattattaaaacaagatgctacgaaagctgaaattaaacaattatgtgatgaagcaattgaatttgattttgcaacagtttgtgttaattcatattgaacaagctattgtaaagaattattaaaaggcacaaatgtaggaataacaaatgttgtaggttttcctctaggtgcatgcacaacagctacaaaagcattcgaagtttctgaagcaattaaagatggtgcaacagaaattgatatggtattaaatattggtgcattaaaagacaaaaattatgaattagttttagaagacatgaaagctgtaaaaaaagcagctggatcacatgttgttaaatgtattatggaaaattgtttattaacaaaagaagaaatcatgaaagcttgtgaaatagctgttgaagctggattagaatttgttaaaacatcaacaggattttcaaaatcaggtgcaacatttgaagatgttaaactaatgaagtcagttgttaaagacaatgctttagttaaagcagctggtggagttagaacatttgaagatgctcaaaaaatgattgaagcaggagctgaccgcttaggaacaagtggtggagtagctattattaaaggtgaagaaaacaacgcgagttactaaaactagcgtttttttattttgctcatttttattaaaagtttgcaaaaaggaacataaaaattctaattattgatactaaagttattaaaaagaagattttggttgattttataaaggtcatagaatataatattttagcatgtgtattttgtgtgctcatttacaaccgtctcgcggccgcgggacgcggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - RNA, 22 nt, synthetic construct
  source 1..22: mol_type = other RNA
tgaggtagtaggttgtatagtt

SEQ ID NO: 3 - RNA, 22 nt, synthetic construct
  source 1..22: mol_type = other RNA
tgaggtagtaggttgtatggtt

SEQ ID NO: 4 - RNA, 22 nt, synthetic construct
  source 1..22: mol_type = other RNA
aacccgtagatccgatcttgtg

SEQ ID NO: 5 - RNA, 21 nt, synthetic construct
  source 1..21: mol_type = other RNA
aacccgtagatccgatcttgt

SEQ ID NO: 6 - RNA, 22 nt, synthetic construct
  source 1..22: mol_type = other RNA
aacccgtagatccgaacttgtg

SEQ ID NO: 7 - DNA, 22 bp, synthetic construct
  source 1..22: mol_type = other DNA
cctagtagaacatgggcatctg

SEQ ID NO: 8 - DNA, 22 bp, synthetic construct
  source 1..22: mol_type = other DNA
gcgatcctagtagttggtgttc

SEQ ID NO: 9 - DNA, 26 bp, synthetic construct
  source 1..26: mol_type = other DNA
agacactggtcttatgaacatgggca

SEQ ID NO: 10 - DNA, 120 bp, synthetic construct
  source 1..120: mol_type = other DNA
gacggcgctaggatcatcaacaactatacaaccaatgtactacctcacaagtattctggtcacagaatacaacaactatacaaccaatgtactacctcacaagatgatcctagcgccgtc

SEQ ID NO: 11 - DNA, 120 bp, synthetic construct
  source 1..120: mol_type = other DNA
gacggcgctaggatcatcttgtgaggtagtacattggttgtatagttgttgtattctgtgaccagaatacttgtgaggtagtacattggttgtatagttgttgatgatcctagcgccgtc

SEQ ID NO: 12 - DNA, 120 bp, synthetic construct
  source 1..120: mol_type = other DNA
gacggcgctaggatcatcaaccacaagatcggaaatgtctacgggtacaagtattctggtcacagaatacaaccacaagatcggaaatgtctacgggtacaagatgatcctagcgccgtc

SEQ ID NO: 13 - DNA, 120 bp, synthetic construct
  source 1..120: mol_type = other DNA
gacggcgctaggatcatcttgtacccgtagacatttccgatcttgtggttgtattctgtgaccagaatacttgtacccgtagacatttccgatcttgtggttgatgatcctagcgccgtc

SEQ ID NO: 14 - DNA, 19 bp, synthetic construct
  source 1..19: mol_type = other DNA
  misc_feature 1..19: note = JRX0111
caagatcggatctacgggt

SEQ ID NO: 15 - DNA, 18 bp, synthetic construct
  source 1..18: mol_type = other DNA
  misc_feature 1..18: note = JRX0112
aagatcggatctacgggt

SEQ ID NO: 16 - DNA, 17 bp, synthetic construct
  source 1..17: mol_type = other DNA
  misc_feature 1..17: note = JRX0114
agatcggatctacgggt

SEQ ID NO: 17 - DNA, 16 bp, synthetic construct
  source 1..16: mol_type = other DNA
  misc_feature 1..16: note = JRX0116
gatcggatctacgggt

SEQ ID NO: 18 - DNA, 15 bp, synthetic construct
  source 1..15: mol_type = other DNA
  misc_feature 1..15: note = JRX0118
atcggatctacgggt

SEQ ID NO: 19 - DNA, 19 bp, synthetic construct
  source 1..19: mol_type = other DNA
  misc_feature 1..19: note = JRX0110
caagttcggatctacgggt

SEQ ID NO: 20 - DNA, 18 bp, synthetic construct
  source 1..18: mol_type = other DNA
  misc_feature 1..18: note = JRX0113
aagttcggatctacgggt

SEQ ID NO: 21 - DNA, 17 bp, synthetic construct
  source 1..17: mol_type = other DNA
  misc_feature 1..17: note = JRX0115
agttcggatctacgggt

SEQ ID NO: 22 - DNA, 16 bp, synthetic construct
  source 1..16: mol_type = other DNA
  misc_feature 1..16: note = JRX0117
gttcggatctacgggt

SEQ ID NO: 23 - DNA, 15 bp, synthetic construct
  source 1..15: mol_type = other DNA
  misc_feature 1..15: note = JRX0119
ttcggatctacgggt

SEQ ID NO: 24 - DNA, 19 bp, synthetic construct
  source 1..19: mol_type = other DNA
  misc_feature 1..19: note = JRX0101
ctatacaacctactacctc

SEQ ID NO: 25 - DNA, 18 bp, synthetic construct
  source 1..18: mol_type = other DNA
  misc_feature 1..18: note = JRX0103
tatacaacctactacctc

SEQ ID NO: 26 - DNA, 17 bp, synthetic construct
  source 1..17: mol_type = other DNA
  misc_feature 1..17: note = JRX0104
atacaacctactacctc

SEQ ID NO: 27 - DNA, 17 bp, synthetic construct
  source 1..17: mol_type = other DNA
  misc_feature 1..17: note = JRX0105
atacaacctactacctc

SEQ ID NO: 28 - DNA, 16 bp, synthetic construct
  source 1..16: mol_type = other DNA
  misc_feature 1..16: note = JRX0106
tacaacctactacctc

SEQ ID NO: 29 - DNA, 16 bp, synthetic construct
  source 1..16: mol_type = other DNA
  misc_feature 1..16: note = JRX0107
tacaacctactacctc

SEQ ID NO: 30 - DNA, 15 bp, synthetic construct
  source 1..15: mol_type = other DNA
  misc_feature 1..15: note = JRX0108
acaacctactacctc

SEQ ID NO: 31 - DNA, 15 bp, synthetic construct
  source 1..15: mol_type = other DNA
  misc_feature 1..15: note = JRX0109
acaacctactacctc

SEQ ID NO: 32 - DNA, 19 bp, synthetic construct
  source 1..19: mol_type = other DNA
  misc_feature 1..19: note = JRX0100
ccatacaacctactacctc

SEQ ID NO: 33 - DNA, 18 bp, synthetic construct
  source 1..18: mol_type = other DNA
  misc_feature 1..18: note = JRX0102
catacaacctactacctc